SEQUENCE LISTING (45 sequences)

SEQ ID NO: 1 - DNA, 1998 bp, Akkermansia muciniphila
  source 1..1998: mol_type = genomic DNA
  CDS 1..1998: product = A. muciniphila beta-N-acetylgalactosaminidase (AmGH20A); protein_id = 2; translation = MKFIIPALALSACLPAAFSQEQIIPKPAEITLFTGSPARLTPDS LIITETQDKAFLDQAGQLQQMLSAGTGLPLPLKPAGQASKKAACIVIKKDPALAARGE EAYSIQSSPSGIILSAADARGIFYAGQSLVQMMPSVFHDRTGDKSAVRWNISETPFRI TDYPRFSWRALMIDEARHFFGEKTIKQIIDQMALLKMNILHWHLTDDTGWRIEIKKYP RLTSIGSKRRESEIGTWNSGKSDGTPHEGFYTQEQIRDIVQYAARRNITIVPEIEMPG HASAAAVAYPFLSLKTPGEVPTTFIVNTAFDPTSEKTYAFLSDVLDEVTAIFPGRIIH IGGDEVRYDKQWKGVPEIEEFMKKNGMKSYADVQMHFTNRMSGIIAQKGRRMMGWNEI YGHDVNGDGGGKAGAKLDTNAVIQFWKGNTSLAKNAIRDGHDVINSLHTSTYLDYSYG SIPLQKAYGFEPVFPGLEKQYHSRVKGLGAQVWTEWISTPERLHYQAFPRACAFAEVG WTPAGKKDFPDFKKRLKAYSERMDLMGIKFARNVISQIDKSDFFNTPRIGTWTPATLT REEHSFDVTKLVKASGKHTVTLLYDKGAHAIEIESVALYENSREVSRDAHAGRSGAHK ENIQYILNAPAPRQGATYTVKANFKGAGGRDSHGTVYFETP
atgaaattcattatccccgcactcgccctgtccgcctgccttcccgcagccttctcacaagaacaaatcatcccgaaaccggcggaaatcacattgttcacaggaagtccggcccgtctcacaccggattccctcatcatcacggaaacacaggacaaagctttcctggatcaggcagggcaattgcagcagatgctcagcgcagggacgggactcccccttccccttaaaccggccgggcaagcgtcgaaaaaagccgcctgcattgtcatcaaaaaagacccagccctggccgccaggggagaagaagcctactccatccaatcatccccaagtggaatcatcctttccgcagccgatgccaggggcatcttttacgcggggcaaagcctggtccagatgatgccctccgtcttccacgaccggacgggggataaatccgccgtccggtggaatatttctgaaactccgttccgcataacggactacccgcgattctcctggcgggcgctgatgattgatgaagcacgccacttctttggcgagaaaaccattaaacagatcatcgaccaaatggctctgctgaaaatgaacatcctgcactggcacctgacggacgacacaggatggcgcattgaaatcaagaagtatccgcgcctgacctccatcggctccaaacgcagggaatcagaaatcggcacatggaacagcggcaagtcagacggaacgccgcatgaaggcttttatacccaggaacagatcagggatatcgtacaatacgcagcccgccgaaacatcaccatcgttccggaaattgaaatgccgggccatgccagcgccgccgccgtagcataccccttcctgagcctgaaaactcccggggaagtgcccacaacattcatcgtcaatacggccttcgatcccacctcggaaaaaacttatgccttcctgtccgatgttttggatgaagtcacggcgatcttccccggcagaatcatccacattggcggagatgaagtgcgctatgacaagcaatggaagggggttccggaaattgaggaattcatgaaaaaaaacggcatgaaaagttatgcggacgtccagatgcatttcaccaaccgcatgtccggcattattgcccaaaaagggcgccgcatgatgggatggaatgaaatttacggacatgacgtcaatggggacggaggaggaaaagccggcgccaaactggatacaaacgccgtcatccagttctggaagggcaacaccagcctggctaaaaacgccatccgggacgggcatgacgttatcaattccctccacacctccacctatttggattacagctacggcagcattcccctgcaaaaggcatacgggttcgaacccgttttccccgggttggagaaacagtaccattccagagtcaagggactgggcgcccaagtatggacggaatggatttccacaccggaacgcctgcactaccaggcattcccccgtgcctgcgcctttgcggaagtcggctggactcccgctggtaaaaaggattttccggatttcaaaaaacgcttgaaagcgtatagcgagcgtatggatctgatggggatcaagtttgcccggaacgtcatcagccaaatagacaaatctgactttttcaatacgcccaggatcggcacatggacgcctgccaccctgacccgggaggaacattcgtttgacgttaccaaactggtcaaagcgtccggcaaacacaccgtcaccctgctgtacgacaaaggcgcccacgccatcgaaatcgaatccgtagccctgtatgaaaattcccgggaagtctcccgggacgcccatgcaggcagaagcggcgcccataaggaaaatatccagtacattctgaatgccccggcccctaggcaaggagcaacctatacggtcaaagcaaacttcaaaggggccggaggccgggattcccacgggacagtgtattttgaaacgccataa

SEQ ID NO: 2 - protein, 665 aa, Akkermansia muciniphila
MKFIIPALALSACLPAAFSQEQIIPKPAEITLFTGSPARLTPDSLIITETQDKAFLDQAGQLQQMLSAGTGLPLPLKPAGQASKKAACIVIKKDPALAARGEEAYSIQSSPSGIILSAADARGIFYAGQSLVQMMPSVFHDRTGDKSAVRWNISETPFRITDYPRFSWRALMIDEARHFFGEKTIKQIIDQMALLKMNILHWHLTDDTGWRIEIKKYPRLTSIGSKRRESEIGTWNSGKSDGTPHEGFYTQEQIRDIVQYAARRNITIVPEIEMPGHASAAAVAYPFLSLKTPGEVPTTFIVNTAFDPTSEKTYAFLSDVLDEVTAIFPGRIIHIGGDEVRYDKQWKGVPEIEEFMKKNGMKSYADVQMHFTNRMSGIIAQKGRRMMGWNEIYGHDVNGDGGGKAGAKLDTNAVIQFWKGNTSLAKNAIRDGHDVINSLHTSTYLDYSYGSIPLQKAYGFEPVFPGLEKQYHSRVKGLGAQVWTEWISTPERLHYQAFPRACAFAEVGWTPAGKKDFPDFKKRLKAYSERMDLMGIKFARNVISQIDKSDFFNTPRIGTWTPATLTREEHSFDVTKLVKASGKHTVTLLYDKGAHAIEIESVALYENSREVSRDAHAGRSGAHKENIQYILNAPAPRQGATYTVKANFKGAGGRDSHGTVYFETP

SEQ ID NO: 3 - DNA, 1650 bp, Akkermansia muciniphila
  source 1..1650: mol_type = genomic DNA
  CDS 1..1650: product = A. muciniphila beta-N-acetylgalactosaminidase (AmGH20C); protein_id = 4; translation = MISKCTFSATVFSLFSLCWGAPSSPVLEAPHTIPLPAAMRVQTG ESGFSLKNGVRLPEKNPLSRQAERIFRDNGINTALVKNNADIIFTEDASLGREGYRLA VTPDSISIASGSVNGTLYALQSLVQSIAADKNGAPALPRMDVKDQPRFSWRGLMVDSC RHMMPVRDIKKVLDLMERYKFNTLHWHLTDDQGWRLPIAKYPRLTTVGGARAQSPVIG NRNKGDGIPYSGHYTADEIRDVVRYARDRGITVIPEVEMPGHASAAIAAYPELGNTDI PGYEPRVQETWGVHSYTFSPTEKTFRFLEDVIDEICALFPDSPYIHIGGDEAPKNQWK QSPTAQRVMKDNGLANEHELQSYFIRRVEKMINNRGKRLIGWDEIQEGGLSPTATMMV WRSQMPHIAAQALAQGNDIVMTPNSHLYFDYDQGPGKPAAPEYETINNNQLTWQHVYG LEPVPQGTPREREKQVLGCQANIWTEYIPNLPKWEYHVFPRALALAEVAWTPQELKNE KDFRKRLDRQLPFLDARGVNYKRPDNGAPAQPKAVITRERR
atgatttccaagtgtaccttttccgccacggttttcagcctgttttccctttgctggggcgccccatcctctccggttctcgaagcgccccataccattcccctgcccgccgccatgcgcgtccaaaccggagaaagcgggttttccctgaaaaacggcgtcaggctcccggaaaaaaatcctctttccaggcaggcggaacggattttccgcgacaacgggatcaacacggccctggttaaaaacaacgcggacatcatctttacggaagacgcttccctgggcagggaaggctaccgccttgccgtaacgccggattccatctccattgcctccggttccgtgaacggaaccctgtatgcccttcaatccctcgttcaaagcatcgctgccgacaaaaacggagctccggccctgccccggatggacgtaaaagaccagccccgcttttcatggcggggcctgatggtagacagctgccgccacatgatgcccgtgcgggacatcaaaaaagtgctggacctgatggaacggtataaattcaacaccctgcactggcacctgacggacgaccaggggtggcgtctcccaatcgccaagtaccccaggctgacaaccgtgggaggcgcccgggctcaatcccccgtcatcggcaaccgcaataagggagacggcatcccctactccggccattacaccgcagatgaaatccgggatgtggtgcggtacgccagagaccggggcattaccgtcattccggaagtggaaatgccaggccatgcctccgcagccatcgccgcctatccggaactggggaatacggacatcccgggttatgagcctagggtgcaggaaacctggggcgtgcactcctataccttctcccccacggaaaaaaccttccgttttctggaagacgtcattgatgaaatatgcgccctgttcccggacagcccctacatccacatcggaggggatgaagcgcccaagaatcagtggaaacagtcccccacggcccagcgggtcatgaaggacaacggcctggccaatgaacacgagctccagagctacttcatccgccgcgtggaaaaaatgatcaataaccgcggaaaaaggctcattggctgggatgaaatccaggaagggggcctttcccccaccgctaccatgatggtttggcgcagccaaatgccgcacatcgccgcacaagccctggctcaaggcaacgatattgtgatgacgcccaacagccacctgtactttgactatgaccaggggcccggaaaacccgctgcccccgaatacgagacgattaataacaatcagctgacctggcagcatgtttacggactggaaccggtgcctcagggaacgccccgggaacgggaaaagcaggtgctgggctgccaggcgaacatctggacggaatatatcccgaacctgccgaaatgggaataccatgtcttcccccgcgccctggcgctggcggaagttgcctggaccccgcaggagctaaaaaatgagaaagatttccgtaaacgcctcgaccgccagcttcccttcctggacgcccgcggcgtcaattacaaaagaccggacaatggagcccccgcacagccgaaggccgtcattacgcgggaacgccgttaa

SEQ ID NO: 4 - protein, 549 aa, Akkermansia muciniphila
MISKCTFSATVFSLFSLCWGAPSSPVLEAPHTIPLPAAMRVQTGESGFSLKNGVRLPEKNPLSRQAERIFRDNGINTALVKNNADIIFTEDASLGREGYRLAVTPDSISIASGSVNGTLYALQSLVQSIAADKNGAPALPRMDVKDQPRFSWRGLMVDSCRHMMPVRDIKKVLDLMERYKFNTLHWHLTDDQGWRLPIAKYPRLTTVGGARAQSPVIGNRNKGDGIPYSGHYTADEIRDVVRYARDRGITVIPEVEMPGHASAAIAAYPELGNTDIPGYEPRVQETWGVHSYTFSPTEKTFRFLEDVIDEICALFPDSPYIHIGGDEAPKNQWKQSPTAQRVMKDNGLANEHELQSYFIRRVEKMINNRGKRLIGWDEIQEGGLSPTATMMVWRSQMPHIAAQALAQGNDIVMTPNSHLYFDYDQGPGKPAAPEYETINNNQLTWQHVYGLEPVPQGTPREREKQVLGCQANIWTEYIPNLPKWEYHVFPRALALAEVAWTPQELKNEKDFRKRLDRQLPFLDARGVNYKRPDNGAPAQPKAVITRERR

SEQ ID NO: 5 - DNA, 1515 bp, Akkermansia muciniphila
  source 1..1515: mol_type = genomic DNA
  CDS 1..1515: product = A. muciniphila beta-N-acetylgalactosaminidase (AmGH20I); protein_id = 6; translation = MVESFPKQFFMMFKLPLILACAIFSAHMACAAAADKYSVIPEPE KTELQHNSTRTLKLLSDQEAPTLGTDAYRLTVTPQGAHLASGGREGRIYGLATLRQLR DQLAGQPEGIPCGVITDKPRYPWRGLMVDPARFFIPTADLKKFVDMMAYYKFNKLQIH LTDDQGWRLPVPGYPKLKSISSKRKESMRNGIPHEGMYTKQELKELVAYCAARGIEVI PEIDVPGHNQALAAAYPEFFCFPNPDTKVKTDEGVTLHLICPHKPEVWKFYAAVFKEL KDIFPSGIVHLGGDEAPLEKTWAKCPLSIQYREQKGMKDVHEELKEFIKKMSSMLAVH GKRIQLWYEKPWARANIYNKGDTVFTWRMGLTPSTITETKKQGLSLIIAAGEYCYLDY PQLPGQSNRGWMPTTTLEQSYRLDPAYGRPEKETNHITGVQGTVWGEHLPTLNHILYR AYPRACAIAEAGWSPMNVRSWENFRRKLADHRQFILKRFNYDMERTKENEPPFK
atggtagaatcttttccgaaacagttcttcatgatgtttaaactgcccctcatcctggcctgcgccattttttccgcccacatggcatgtgccgccgcagcggacaaatacagcgttatccctgaaccggaaaaaacggagctgcagcacaacagtaccagaaccttaaaacttctttccgaccaggaggctccgaccctgggaacggacgcctaccggctcacagtcaccccgcagggggcgcaccttgcttccggaggaagggaaggcagaatttacgggctggcaaccctccgccagctccgggaccagctggcgggacagccggagggcatcccctgcggcgtcatcacggacaagccgcgctatccgtggcgcggcctcatggtagatcccgcacgctttttcatccccacggccgatctgaaaaaatttgtggatatgatggcctactacaaattcaacaagctccagatccacttgacggacgaccaggggtggcgtcttccggtgcccggctaccccaaactcaaaagcatctcctccaaacggaaagaaagcatgcgcaacggaatcccccatgaagggatgtacaccaaacaggaactgaaagagctggtggcgtactgcgcagcgcgcggcattgaggtcatccctgaaatagacgtgccggggcacaaccaagccctggcggcagcctaccctgaattcttctgcttcccgaacccggatacgaaagtgaagaccgatgaaggcgtcaccctccacctcatctgcccgcataaaccggaagtctggaaattttatgccgctgttttcaaggaactcaaagatattttcccgtccggcatcgtccatctgggcggcgatgaagcacccctggaaaaaacctgggccaaatgccccctcagcatccagtaccgggagcaaaaaggcatgaaggacgtccacgaggaattgaaggaattcatcaaaaaaatgtcctccatgctggctgttcacggcaagcgcatccaactatggtatgaaaaaccgtgggccagggccaacatctacaacaaaggagacaccgtcttcacctggcgcatgggactgacaccgtccaccatcacggagacgaaaaagcaggggctctccctgatcattgctgccggggaatactgttacctggactatccgcaacttccagggcaaagcaaccggggatggatgcccaccaccacgctggagcaaagctacaggctggaccccgcctacggcagaccagaaaaggaaacaaaccatatcaccggcgttcagggcaccgtgtggggagaacatctccctaccctgaaccacattctctaccgcgcctatccgcgtgcctgcgccattgcggaagccggctggtcaccgatgaacgtgcgctcctgggaaaacttccggcgcaagctggccgaccaccgtcaattcatcctcaaacgcttcaattatgatatggagcgcaccaaagaaaacgaaccgcctttcaaataa

SEQ ID NO: 6 - protein, 504 aa, Akkermansia muciniphila
MVESFPKQFFMMFKLPLILACAIFSAHMACAAAADKYSVIPEPEKTELQHNSTRTLKLLSDQEAPTLGTDAYRLTVTPQGAHLASGGREGRIYGLATLRQLRDQLAGQPEGIPCGVITDKPRYPWRGLMVDPARFFIPTADLKKFVDMMAYYKFNKLQIHLTDDQGWRLPVPGYPKLKSISSKRKESMRNGIPHEGMYTKQELKELVAYCAARGIEVIPEIDVPGHNQALAAAYPEFFCFPNPDTKVKTDEGVTLHLICPHKPEVWKFYAAVFKELKDIFPSGIVHLGGDEAPLEKTWAKCPLSIQYREQKGMKDVHEELKEFIKKMSSMLAVHGKRIQLWYEKPWARANIYNKGDTVFTWRMGLTPSTITETKKQGLSLIIAAGEYCYLDYPQLPGQSNRGWMPTTTLEQSYRLDPAYGRPEKETNHITGVQGTVWGEHLPTLNHILYRAYPRACAIAEAGWSPMNVRSWENFRRKLADHRQFILKRFNYDMERTKENEPPFK

SEQ ID NO: 7 - DNA, 2385 bp, Akkermansia muciniphila
  source 1..2385: mol_type = genomic DNA
  CDS 1..2385: product = A. muciniphila alpha-galactosidase (AmGH110A); protein_id = 8; translation = MEGNLSFSLMEASGRSIFFLIEGIREQSIKNMFSRMFSWSFVVA ACLAGLFPAQSQGEEKAAQSGTIAVKVPASSLLMTRQETGETRLDRSFSNAGLSIGGK KYATGIGTHATSMIPLPVPENPKVLRLEGACGIDDGADGDGSVEFRVMSGSEVLWSSG VMRRGMAAKKFSIPVAENGIRHLYLMADRVDNNSYDHADWVDLAWKTTGSGQGMKGAV VNASEFGMVPGVRKDQGPALRAAVSALRRQGGGVLNIPRGIYHFYPEGALNMSFHISN HDQPLIHPVCVPLADLRNVRVEGNGSLFLFHGKVVPLLVMDSENVSINRLSVDYERSW CTEARVVKTDDRFTEVEIDKKAYPYEIRNNRFVFQGKGWEEGMGSCMAFEKGTGHIIA NTSDIGWNGHVEPLGGSRLRLSWNLRQKGIKPGDTLVLRNYNRPHPGCVVYRARKTSL NDVSLHQSSGMALLVQRSEDFHMKGGGVMVRKGTGRVHTAGADATHFSNTRGGIVVEK ALFEGMMDDAINVHSTCLGVMEVVDSHTLKCKYMHRQAVGFEVFLPGEKIRFINGPTL EPGGTATVKTAVKKNSAEMVITVEEPLPSSVRAGDAVENADFYPSVVFRNNIVRNNRA RGSLFTTPERVLVEGNLFDHSSGSAILLAGDAQGWYESGACHEVVIRKNTFINNLTSR YQFTNAIISIYPEVKQLDRQRDYYHRNVLIENNVFKTFDVPLLFAISTDNLKFINNKV IYNDEFKGWGQKPFQFRRCANILIKDNKVLPPRTWTLEDCKLENTPSDQVRFGG
atggaaggcaatctctctttttctctgatggaagcttctggacgatccatattttttctgatagaaggaatacgggaacagagtataaaaaatatgttcagccggatgttttcatggagttttgtagttgcagcgtgtttggcgggattattcccggcacagtcccagggcgaggaaaaagctgcccaatccggaaccatagccgtaaaagttcctgcctccagcctgctgatgactcgccaggagacgggggaaacccggttggaccggtctttcagtaatgcggggttgagcataggcggcaaaaaatatgccacgggaatcggtacgcacgctacgtccatgattcccctgcccgttccggaaaacccgaaagttttgaggctggaaggtgcatgcggaattgatgacggtgcggatggagacggaagcgtggaattccgcgtgatgagcgggtccgaggttttatggagttccggcgtgatgaggagggggatggccgcgaagaagttttccattccggtggcggaaaacggcatacggcatctttacctgatggccgaccgggtggacaataattcttacgaccatgcggactgggtagatctggcctggaagacgacaggaagtgggcaggggatgaagggagctgtggtgaatgcctctgaattcggcatggttcccggcgtcagaaaagatcaggggccggcgctgcgggctgccgtttccgctctccggaggcagggcgggggcgttttgaacatccccagaggcatttaccatttttatccggagggggctttgaacatgagtttccatatttccaaccatgaccagccgctgattcatccggtgtgtgtgcctctggccgatttgcggaatgtccgcgtggagggcaatggttccctgtttctttttcacggcaaggtggttcccctcctggtgatggacagcgaaaatgtcagcatcaaccgtttatccgtggattacgaacggtcctggtgcacggaggcgcgcgtcgtgaagacggatgaccggttcacggaagtggaaatagacaaaaaggcctacccttacgaaatccggaacaacaggttcgttttccaggggaaaggctgggaagaaggaatgggaagctgcatggcctttgagaaagggacgggacatattatcgccaatacatcggacatcggctggaacgggcatgtggagccgctgggcgggagccgtctccgtctgtcctggaacctcaggcagaagggaatcaagccgggggatacgctggttcttcggaattataaccgcccccatccgggatgcgtggtgtaccgggctcggaaaacatcactgaatgacgtatctctgcaccagagctcaggcatggccctgctggtccagcgatcggaggactttcatatgaaaggcggcggagtcatggtcaggaaaggcacagggcgcgtacacaccgccggagcggacgcaacccatttttccaatacccggggcggaatcgtggtggagaaagccctgttcgaagggatgatggatgatgccatcaatgttcattccacctgcctgggcgtgatggaagtagtggacagccataccctgaagtgcaaatacatgcatcggcaggcggtggggtttgaggtgtttcttcccggtgaaaaaatccgtttcatcaacggtcctacgctggaacccggcggaacggccactgtgaagacggcagtgaaaaagaattccgcggagatggtgattacggtggaagagccgctcccctcctccgtcagggccggggatgccgtggagaatgcggatttttacccttccgtggttttccgcaacaatatcgtccgcaacaaccgtgccagaggatctctttttacaacgccggagagagtgctggtggagggcaatttgtttgaccattcctccggctccgccattctattggcaggggatgcccaggggtggtatgaaagcggcgcctgccatgaagtggtgatccgcaaaaatacattcattaacaacctgacctcgcgttaccagtttacgaatgccatcatttccatttacccggaagtcaagcagctggacaggcagagggattactatcatcgcaatgtgctgatagaaaataatgttttcaagacgttcgatgtgccgctgctgttcgccatttccacggacaacctcaagttcatcaataataaggtcatttacaatgacgagtttaagggatgggggcagaaacctttccaattcagaagatgcgccaatattctgattaaagataacaaggtgctgcctccccgcacatggacccttgaggactgcaagctggaaaataccccatcagatcaggtccgctttggtggataa

SEQ ID NO: 8 - protein, 794 aa, Akkermansia muciniphila
MEGNLSFSLMEASGRSIFFLIEGIREQSIKNMFSRMFSWSFVVAACLAGLFPAQSQGEEKAAQSGTIAVKVPASSLLMTRQETGETRLDRSFSNAGLSIGGKKYATGIGTHATSMIPLPVPENPKVLRLEGACGIDDGADGDGSVEFRVMSGSEVLWSSGVMRRGMAAKKFSIPVAENGIRHLYLMADRVDNNSYDHADWVDLAWKTTGSGQGMKGAVVNASEFGMVPGVRKDQGPALRAAVSALRRQGGGVLNIPRGIYHFYPEGALNMSFHISNHDQPLIHPVCVPLADLRNVRVEGNGSLFLFHGKVVPLLVMDSENVSINRLSVDYERSWCTEARVVKTDDRFTEVEIDKKAYPYEIRNNRFVFQGKGWEEGMGSCMAFEKGTGHIIANTSDIGWNGHVEPLGGSRLRLSWNLRQKGIKPGDTLVLRNYNRPHPGCVVYRARKTSLNDVSLHQSSGMALLVQRSEDFHMKGGGVMVRKGTGRVHTAGADATHFSNTRGGIVVEKALFEGMMDDAINVHSTCLGVMEVVDSHTLKCKYMHRQAVGFEVFLPGEKIRFINGPTLEPGGTATVKTAVKKNSAEMVITVEEPLPSSVRAGDAVENADFYPSVVFRNNIVRNNRARGSLFTTPERVLVEGNLFDHSSGSAILLAGDAQGWYESGACHEVVIRKNTFINNLTSRYQFTNAIISIYPEVKQLDRQRDYYHRNVLIENNVFKTFDVPLLFAISTDNLKFINNKVIYNDEFKGWGQKPFQFRRCANILIKDNKVLPPRTWTLEDCKLENTPSDQVRFGG

SEQ ID NO: 9 - DNA, 2538 bp, Akkermansia muciniphila
  source 1..2538: mol_type = genomic DNA
  CDS 1..2538: product = alpha-1,3-N-acetylgalactosaminidase (AmGH36A); protein_id = 10; translation = MKFFKTTIAMATLLGCLPTSPAEVAFPGPAPGTASAEQSASGYT LSNNILTAKFILRNGSLSFGGMTSKLGPVAKAGDELFIINLQDGRSIKSSELKAGNVK FVSLQPNPKAFRLADRFPGKAVSATFQALNGTLRISWRAVLRDHSHYLRQELRLASMK PVQMKNIVAMQYDLAEGKAGAPSVSGNARGALIVNDLAFTALETPMGINTVGGAGNME GGETAIWSADKWSTASWTGNFNVPQELKKQYGEQVSSAEGPVSIDGKGACTVTFQYKG GDKGNNKLNLAGVQLLSSKGAVLDSDFHNGSTGDSNAGNTYTVQVPSRGNYILRYWAQ TKSEPIASKGEITFSLPVRTLEEKQEPQADESRLAQGLWSRKTTLQPGEIWDVSSVLG IFAPGQKRRSFLAYMERERVMPYRPFIHYNSWYELNINRNNDSDPAKRMTEEQCLAVL KDWQEQFFQKRGMSIDAFVWDDGWDEFNSLWDFHKMFPQGFKRIDAAAGRQKAGIGTW LGPVGGYGASKGKRLAYWNVKHPDNKIGNFQLSNKEYFDAFVGRCSQMVKDYNMKYFK FDGISTHFHAKGPGNEEDAEGIIRVLNALRKKKGDLYINCTVGTWASPFWFRYADSVW RQENDFGTIGAGDNRDKWITYRDRLVHEVFVQGSPLMPINSMMTHGLMVTKFGPPACM PRDPENVKKELRCATACGTSLQELYVDRDLMSANGGVLWDELAKGIKWIRRNADVLDD VHWVGGNPWNKETNEGAVYGWAAWNKNKATLALRNSSDQEKSLTGTLRSILDIPANVK GSITFKDSYDDQRTLDGFSGSSVNIDKEISFTLKPFEVLVYEGGKVK
atgaaatttttcaagacaaccattgccatggcaaccctgctgggctgcctgcccacatcccccgcagaagtcgcctttccggggcctgctcccggaacagccagcgcggaacaatccgcttccggctatactctttccaataacatcctgacggccaaatttatccttcggaacggaagtctttccttcggaggaatgacttccaaactgggccccgtcgccaaggcaggggatgaattattcatcatcaatcttcaggacggacggagcattaaatcctccgagctgaaagcgggaaacgtcaaattcgtctcccttcagcccaaccccaaggccttcaggctggctgaccgcttcccgggaaaagccgtcagcgcaacattccaggctctgaacggaacccttcgcatttcctggcgcgccgtgctcagggaccattcccattacctgaggcaggaactccgcctggcaagtatgaagcccgtccagatgaaaaacatcgtagcgatgcaatatgacctggcagaagggaaagccggggcaccctccgtttccggaaacgccagaggagcactcatcgtcaatgacctggcgttcaccgccctggaaacacccatgggcatcaataccgtgggaggcgccggaaatatggaagggggagaaaccgccatctggagtgcggacaagtggtccacagcttcatggaccggaaatttcaatgttccccaggaactgaaaaagcagtacggagaacaggtttccagtgcggaaggcccggtttccattgacggcaaaggagcctgcaccgtcaccttccagtataagggaggggacaagggcaacaataagctgaaccttgccggcgtgcagctcctctcttccaagggggccgttcttgactccgatttccacaacggcagcaccggagattccaatgccggcaatacttacacggtgcaggttccctccaggggaaattacattctgcgctactgggcccagaccaagagcgaaccgattgccagcaagggggaaatcaccttttccctgcccgtgcgcacgctggaggaaaagcaggaaccccaggcggacgaatcccggctggcccagggactctggagccgtaaaaccaccttgcagcccggggaaatatgggacgtatcctccgttctcggaatttttgcccccggccagaaacgtcgctccttcctggcctatatggaacgggaacgcgtgatgccctaccgccccttcatccattataattcctggtatgaactgaacatcaaccgcaacaatgattcggaccccgccaagcgcatgacggaagaacaatgcctggccgtcctgaaagactggcaggaacagtttttccagaagcgcggcatgtccattgacgccttcgtctgggacgacggctgggatgaattcaacagcctgtgggatttccacaaaatgttcccgcagggcttcaagcgcattgacgccgccgccggcaggcaaaaggccggcatcggcacgtggctgggtccggtggggggctacggagcctccaagggcaagcgtctcgcttactggaacgtgaagcatccggacaacaagatcggtaatttccagctctccaacaaggaatattttgacgcctttgtgggccgctgttcccagatggtgaaggactacaatatgaagtacttcaaattcgacggcatcagcactcacttccatgccaaggggcccggcaacgaggaggatgcggaaggcatcatccgcgtgctgaacgccctgcgcaagaaaaagggggatctttacatcaactgcactgtaggaacctgggcatcccccttctggttccgttatgcggattccgtatggaggcaggagaatgacttcggcaccatcggagcgggcgacaaccgcgacaaatggatcacctaccgcgaccgcctggtgcatgaagtttttgtgcagggttccccgctgatgcccatcaattccatgatgactcacggcctgatggtcactaaattcggtcctccggcctgcatgccccgcgatccggaaaacgtcaagaaagaactccgctgcgccacggcttgcggcacttccctccaggagctctacgtggatcgggacctgatgagcgccaacggcggagttctgtgggacgaactggccaaggggatcaagtggatacgccgcaatgcggacgttttggacgatgtgcactgggtgggcggcaatccgtggaacaaggaaactaacgaaggggccgtgtacgggtgggccgcatggaataaaaacaaggccaccctggctctccgcaattcctccgaccaggaaaagagcctgaccggaaccctgcgcagcattctggatatccctgcgaacgtcaagggctccatcaccttcaaggattcctatgacgaccagcggacgctggacggcttctccggcagttccgtaaacattgacaaggaaatctccttcacgttgaagccctttgaggtgctggtgtacgaagggggaaaggtcaaataa

SEQ ID NO: 10 - protein, 845 aa, Akkermansia muciniphila
MKFFKTTIAMATLLGCLPTSPAEVAFPGPAPGTASAEQSASGYTLSNNILTAKFILRNGSLSFGGMTSKLGPVAKAGDELFIINLQDGRSIKSSELKAGNVKFVSLQPNPKAFRLADRFPGKAVSATFQALNGTLRISWRAVLRDHSHYLRQELRLASMKPVQMKNIVAMQYDLAEGKAGAPSVSGNARGALIVNDLAFTALETPMGINTVGGAGNMEGGETAIWSADKWSTASWTGNFNVPQELKKQYGEQVSSAEGPVSIDGKGACTVTFQYKGGDKGNNKLNLAGVQLLSSKGAVLDSDFHNGSTGDSNAGNTYTVQVPSRGNYILRYWAQTKSEPIASKGEITFSLPVRTLEEKQEPQADESRLAQGLWSRKTTLQPGEIWDVSSVLGIFAPGQKRRSFLAYMERERVMPYRPFIHYNSWYELNINRNNDSDPAKRMTEEQCLAVLKDWQEQFFQKRGMSIDAFVWDDGWDEFNSLWDFHKMFPQGFKRIDAAAGRQKAGIGTWLGPVGGYGASKGKRLAYWNVKHPDNKIGNFQLSNKEYFDAFVGRCSQMVKDYNMKYFKFDGISTHFHAKGPGNEEDAEGIIRVLNALRKKKGDLYINCTVGTWASPFWFRYADSVWRQENDFGTIGAGDNRDKWITYRDRLVHEVFVQGSPLMPINSMMTHGLMVTKFGPPACMPRDPENVKKELRCATACGTSLQELYVDRDLMSANGGVLWDELAKGIKWIRRNADVLDDVHWVGGNPWNKETNEGAVYGWAAWNKNKATLALRNSSDQEKSLTGTLRSILDIPANVKGSITFKDSYDDQRTLDGFSGSSVNIDKEISFTLKPFEVLVYEGGKVK

SEQ ID NO: 11 - DNA, 1446 bp, Akkermansia muciniphila
  source 1..1446: mol_type = genomic DNA
  CDS 1..1446: product = alpha-1,3-N-acetylgalactosaminidase (AmGH109B); protein_id = 12; translation = MDNSSSRRRFLQTLGLATGALAAGSFANAQEVAPLAPKKITIPD PNNIGPMTTWPPRKPGAIYMGGFRAPKLDKVRVAFVGVGERGSMHVGQMAVIEGAEIV GICDLYEDWAKRSADVVEKKTGKRPPIFTKGPEDYKRMMKEVKPDAVIVCPSWEWHCR VTCDVMKMGAHAFVEVPMAVSIKELWEIVDTSEETRKHCMMMENVNYGREELMYLNMV RQGVIGDLLYGEAAYIHELRGQMKQVERGTGSWRTYHYAKRNGNVYPTHGLGPIAQYM NLARKDDCFGRLVSFSSPALGRAAYAKKNFPADHKWNKLDFACGDMNTSIIKTTMGRT VLVEWDETSPRPYSRLNLIQGTLGTLAGFPTRVAGEKLGNGNYHEWIEGKEKLAPIFE KYDHPLWKRIGPLALKMGGHGGMDFVMLFRIIECLRNGEPMDQNVYEGAFWSSVSELS EYSVAQGGMPQVFPDFTRGDWKTTAPLGIVQ
atggataattcatcatcacgccgtcgtttccttcagaccctgggcctggccactggcgccctggctgccggttcctttgccaacgcccaggaagtagcccccctggctcccaagaaaatcaccattccggacccgaataacatcggccccatgaccacgtggcccccgcgcaagcccggcgccatctacatgggcggcttcagggctcccaagctggacaaggtgcgtgtggcctttgtcggcgtgggtgaacgcggttccatgcacgtgggccagatggccgttatagaaggtgcggaaattgtcggcatttgcgacctgtatgaagactgggccaagcgcagtgcggacgtcgtggaaaagaagacgggcaagcgcccccccattttcacgaaaggaccggaagactacaagcgcatgatgaaggaagtcaagccggacgccgtcatcgtctgccccagctgggaatggcactgccgtgttacctgcgacgtgatgaagatgggcgcccacgcctttgtggaagtgcctatggccgtctccatcaaggaactctgggaaatcgtggatacctccgaagaaaccaggaagcactgcatgatgatggaaaacgtcaactacggacgtgaggaactcatgtacctgaacatggtgcgccagggcgtcattggcgacctgctgtacggagaagccgcctacatccatgaactgcgcggacagatgaagcaggtggaacgcggcaccggttcctggagaacctatcactacgccaagcgcaacggcaacgtgtatcccacgcacggcctcggccccattgcccagtacatgaatctggcccgcaaggacgactgcttcggcaggctcgtctccttctccagcccggccctgggccgcgccgcgtatgccaagaaaaatttcccggcggaccacaagtggaacaagctggactttgcctgcggcgatatgaatacctccatcatcaagaccaccatgggccgcaccgtcctggtggaatgggatgaaaccagtccgcgcccctactcccgcctgaatctcatccagggcaccctgggcaccttggccggcttcccgacccgcgtagccggggaaaagctgggcaacggaaattatcatgaatggattgaaggcaaagaaaaactggcccctatttttgaaaagtacgatcacccgctctggaagagaatcgggccgctggccctgaagatgggcggtcacggcggcatggacttcgtgatgctcttccgcatcatcgaatgcctccgcaatggcgaaccgatggaccagaacgtttatgaaggagctttctggtcctccgtctccgagctttccgaatactccgtggcccagggcggcatgccccaggtattcccggacttcacccgcggagactggaaaacgactgctccgctgggcatcgtccagtaa

SEQ ID NO: 12 - protein, 481 aa, Akkermansia muciniphila
MDNSSSRRRFLQTLGLATGALAAGSFANAQEVAPLAPKKITIPDPNNIGPMTTWPPRKPGAIYMGGFRAPKLDKVRVAFVGVGERGSMHVGQMAVIEGAEIVGICDLYEDWAKRSADVVEKKTGKRPPIFTKGPEDYKRMMKEVKPDAVIVCPSWEWHCRVTCDVMKMGAHAFVEVPMAVSIKELWEIVDTSEETRKHCMMMENVNYGREELMYLNMVRQGVIGDLLYGEAAYIHELRGQMKQVERGTGSWRTYHYAKRNGNVYPTHGLGPIAQYMNLARKDDCFGRLVSFSSPALGRAAYAKKNFPADHKWNKLDFACGDMNTSIIKTTMGRTVLVEWDETSPRPYSRLNLIQGTLGTLAGFPTRVAGEKLGNGNYHEWIEGKEKLAPIFEKYDHPLWKRIGPLALKMGGHGGMDFVMLFRIIECLRNGEPMDQNVYEGAFWSSVSELSEYSVAQGGMPQVFPDFTRGDWKTTAPLGIVQ

SEQ ID NO: 13 - DNA, 2391 bp, Akkermansia muciniphila
  source 1..2391: mol_type = genomic DNA
  CDS 1..2391: product = alpha-1,2-fucosidase (AmGH95B); protein_id = 14; translation = MHFQSLYLSAVAVSAVSFGWGALDKPSASNLIWSDEPAVVVYPQ EDKNSEGSFGKYRKPASVWEAEGYPIGNGRVGAMIFSAPGRERLALNEISLWSGGANP GGGYGYGPDAGTNQFGNYLPFGDLFVDFKKGDQPASLSVEDFTRSLDLRDGIHKVNYK ADGVTYDREAFSSTPANVLVLNYKASKPGQFSADFSVNSQLGADISAKGSVITWKGML KNGMNYEGRVLIRPKGGTLSASGDKISVKNADSCMVVIAMETDYLMDYKKDWKGESPS RKLDRYAAKAASADYAALKQAHISQYKSMFDRVKVNFGKTEEDVAKLPTPKRLEAYKK NPADPDLEETMFQFGRYLLLSSSRPGTLPANLQGLWNDYVKPPWACDYHNNINVQMAY WGAEPANLSECHEALVNYVEAMAPGCRDASQANKGFNTKDGKPVRGWTVRTSQNIFGG NGWQWNIPGAAWYALHIWEHYAFTGDRKYLEKQAYPLMKEICHFWEDHLKELGAGGEG FKTNGKDPSEEEKKDLADVKAGTLVAPNGWSPEHGPREDGVMHDQQLIAELFSNTIKA ARILGKDAAWAKSLEGKLKRLAGNKIGKEGNLQEWMIDRIPKTDHRHTSHLFAVFPGN QISKLKTPKLAEAARLSLEWRGTTGDSRRSWTWPWRTALWARLGEGNKAHEMVQGLLK FNTLPNMLTTHPPMQMDGNFGIVGGICEMLVQSHAGGLDIMPSPVEAWPEGSVKGLKA RGNVTVDFSWKDGKVSNVKLYSAQPKVLPVRVNGKMTRMKTLPLKSGAGSSQPAAR
atgcatttccaatctctatatttatctgccgtggcggtaagtgccgtttctttcgggtggggggctctggataagccttccgcctccaatttgatctggtctgatgaaccggccgtagttgtttatccgcaggaagacaaaaattccgagggcagttttggcaagtacagaaagcctgcctccgtctgggaagcggaggggtatcccattggcaatggtcgcgttggagccatgattttcagcgctcccggccgcgagcggctggccctgaatgaaatcagcctttggtccggaggtgccaatccggggggagggtacggttacgggcctgatgccggaacgaaccagttcggcaactatcttccctttggggatttgtttgtggactttaaaaaaggcgaccaaccggcttccctgtctgtggaagattttacgcgctccctggatctccgggacggcattcataaagtgaattacaaggcggacggcgtaacgtatgaccgggaggcattctccagcacgcctgccaacgtcctggtgctgaattataaagccagcaaacccggccaattcagcgcggatttttccgttaacagccagcttggagcagatatttccgccaagggatccgtcatcacctggaaggggatgttgaaaaacggcatgaattatgaaggccgcgttttgatccgtcccaaaggcggtacgctttctgcctcgggagataaaatttccgtgaaaaatgcggattcctgcatggtcgtcatcgccatggagacggattacctgatggattataaaaaggactggaagggtgaatctccctccaggaagctggaccgttatgcggccaaagccgcttctgcggattatgccgccctgaaacaggcccacatttcccagtacaagtccatgtttgaccgggtgaaggtcaacttcggaaaaacggaggaggatgtagccaagctgcctacgccaaaacgtctggaggcctataagaaaaatccggcagaccccgatttggaggaaaccatgttccagtttggccgatatctgctgttgtccagttcccggcccggcacgctaccggccaacctgcaagggttgtggaacgattatgtcaaaccgccgtgggcctgcgactaccataacaacatcaacgtccagatggcgtattggggggcggaacccgccaatctttccgaatgccatgaggccctggtcaattatgtggaggcaatggcccccggctgccgggacgcttcccaggcgaacaaggggttcaataccaaggacggtaaacccgtgcgcggctggacggtgcgcacctcccagaatatcttcggtggcaacggctggcagtggaacattcccggtgcggcctggtatgcgctgcatatatgggaacattatgcatttaccggtgatagaaagtatctggaaaaacaggcgtatcccctgatgaaggaaatctgtcatttctgggaagaccatttgaaggaactgggagccgggggcgaagggttcaagacaaacggcaaggatccgagcgaagaggagaagaaggatctggccgatgtgaaagccggaactctggtggctcccaatggctggtctcccgaacatgggccgcgtgaagacggtgtgatgcatgaccagcagctcattgcggagcttttctccaataccatcaaagccgcccgcattctgggcaaggacgccgcctgggccaagagcctggagggcaagctgaaaaggctggccggcaacaagataggcaaggaagggaatcttcaggaatggatgattgaccgcattcccaagacggaccaccgccatacgtcccacctttttgccgttttccccggcaaccagatcagcaagctcaagacgcccaagctggcggaagccgcccgcctttccttggaatggcgcggcacgaccggagacagccgccgttcctggacgtggccgtggcgcacggctctgtgggcccgcctgggcgaggggaacaaggctcatgaaatggttcagggactcttgaaattcaacactttgccgaatatgctgactactcatccccctatgcagatggacggcaacttcggcattgtaggcggcatttgtgaaatgttggtgcaatcccatgccgggggcctggacatcatgccctctcccgtggaagcgtggcctgaaggttccgtgaaagggctgaaagcccggggcaacgtcacggtggatttttcctggaaggacggcaaggtaagcaacgtgaagttgtattccgcccaacccaaggtgttgcccgtgcgcgttaacgggaagatgacccgcatgaaaaccctgccgctgaaatcgggagcgggatcttcgcagcccgcggccaggtaa

SEQ ID NO: 14 - protein, 796 aa, Akkermansia muciniphila
MHFQSLYLSAVAVSAVSFGWGALDKPSASNLIWSDEPAVVVYPQEDKNSEGSFGKYRKPASVWEAEGYPIGNGRVGAMIFSAPGRERLALNEISLWSGGANPGGGYGYGPDAGTNQFGNYLPFGDLFVDFKKGDQPASLSVEDFTRSLDLRDGIHKVNYKADGVTYDREAFSSTPANVLVLNYKASKPGQFSADFSVNSQLGADISAKGSVITWKGMLKNGMNYEGRVLIRPKGGTLSASGDKISVKNADSCMVVIAMETDYLMDYKKDWKGESPSRKLDRYAAKAASADYAALKQAHISQYKSMFDRVKVNFGKTEEDVAKLPTPKRLEAYKKNPADPDLEETMFQFGRYLLLSSSRPGTLPANLQGLWNDYVKPPWACDYHNNINVQMAYWGAEPANLSECHEALVNYVEAMAPGCRDASQANKGFNTKDGKPVRGWTVRTSQNIFGGNGWQWNIPGAAWYALHIWEHYAFTGDRKYLEKQAYPLMKEICHFWEDHLKELGAGGEGFKTNGKDPSEEEKKDLADVKAGTLVAPNGWSPEHGPREDGVMHDQQLIAELFSNTIKAARILGKDAAWAKSLEGKLKRLAGNKIGKEGNLQEWMIDRIPKTDHRHTSHLFAVFPGNQISKLKTPKLAEAARLSLEWRGTTGDSRRSWTWPWRTALWARLGEGNKAHEMVQGLLKFNTLPNMLTTHPPMQMDGNFGIVGGICEMLVQSHAGGLDIMPSPVEAWPEGSVKGLKARGNVTVDFSWKDGKVSNVKLYSAQPKVLPVRVNGKMTRMKTLPLKSGAGSSQPAAR

SEQ ID NO: 15 - DNA, 1932 bp, Akkermansia muciniphila
  source 1..1932: mol_type = genomic DNA
  CDS 1..1932: product = beta-1,3-galactosidase (AmGH35A); protein_id = 16; translation = MMKQYGFSWSAALMAVGVGALAWAGPEAVQNVQKPALSGGPPVV FGFGGEGNQEFMLNGKPFQIRGAEMHPQRIPREYWRHRIRTAKAMGLNTIAFYVFWND HEQPDGSFDFKTGNRDLEGFLKLCQEEGMWVLFRPGPYACGEWDLGGLPHYLLKDPKA KLRTTEDAKFMKAQTRYLEAVARVAEPFLAKNGGPILMTQLENEYGSYQRKDRKYMEW LKAFWSRKGFGPFYTSDGAGEHFLKGVVLPGVAVGLDPGLNDGHWAVANKCNPGVPVF SSETYPGWLRHWGEGNWAPTPGVVNHVRWFMDKGRSFSLFVFHGGTNFGFSAGANNGG PGKYQPDLTSYDYGSPVDEQGRMNEYYAQMREIILKKLPPEAAVPEPPADIPAMEIPE FTPAVHAGLWENLPKPFRSKFPQPPYFEQWNQNQGIAVYRTAVPSGPPETLEFTNVND YAQVYLDGELVGTLDRRLGQKSVKLPERRKPGTLEVLVEAMGHINFHISMESDRKGIY GPVKLGTRELKNWTVRALPLKADSIVRAPKGKGPSQKREGAHFRAVVNIEEPQDTFLD MSRYVKGYVWVNGINVGRYWNVGPQLRLYVPAPFLKKGENVIDILDLHEKEPKPVRGM KERNKEPGKINTKNLDNQW
atgatgaagcaatatggtttcagttggtctgccgcactgatggccgtcggagtgggagcattggcatgggctggccctgaggctgtccagaatgtacagaaacccgctctttccggaggaccccccgttgtgttcgggttcggcggggaagggaaccaggagttcatgctgaacgggaaacccttccagatccgcggcgcggagatgcatccccagcgcattccccgggaatactggaggcaccgcatcaggaccgccaaggctatggggctgaatactattgcattttatgtgttctggaatgaccatgagcagccggacggcagctttgactttaagacgggcaatcgggatctggaagggttcctcaagttatgccaggaggaaggaatgtgggttttattccgccccggcccctatgcatgcggggaatgggatctgggtgggctgcctcattatttgctgaaggatcccaaggccaagttgagaactacggaagacgccaaattcatgaaggcgcagacgcgttatctggaggccgtggctcgtgtggcggagccttttttagccaaaaacgggggccccattctgatgacccagcttgaaaacgaatacgggagctaccagcgtaaagaccgcaagtatatggaatggctgaaggcgttctggagcaggaagggttttggccccttttacacctccgacggcgcgggagaacattttctgaaaggcgtggtgcttccaggcgtggccgtagggctggatccggggctgaacgacggccattgggcagtggctaataaatgcaatccgggagttcccgttttttcctcggaaacatatccgggttggctgcggcactggggggaggggaattgggctcccacccctggagtggtcaaccacgtccgctggtttatggacaaggggcgttccttcagcttgttcgttttccacggaggcaccaatttcggattctcggccggagccaacaacggagggccgggaaaataccagccggacctgacgagttatgattacggttctcccgtggatgagcaggggcggatgaatgaatattatgcccagatgagggaaatcattttgaaaaagttgcctcccgaagccgctgtgccggaacctcccgcagacattccggccatggaaattccggagttcacgcccgcagtgcatgccggcctttgggagaacctgcccaagcctttccggtccaagttcccgcagcctccctattttgaacaatggaaccagaaccagggtattgccgtttacagaacggccgttccgtcaggaccgcctgaaacgctggaatttaccaatgtcaatgactatgcccaggtgtatctggatggagagctggtcggcacgctggatcggcggctggggcagaagagcgtgaaactgccggagcgcaggaagccggggacgctggaagttctggtagaggccatgggacatattaattttcatatcagcatggagagtgaccgcaaggggatttacggtcctgtgaagctgggaacgcgggagttaaagaactggacggtgagggcgcttcccctgaaagctgattccattgtgcgggctcccaaaggaaaggggccttcccagaaacgggaaggggcgcatttccgggccgttgtaaatattgaagagcctcaggacacgtttctggatatgtcccgctatgtcaaggggtatgtatgggtgaacggaatcaacgtggggcgctattggaatgtgggacctcagttaaggctgtatgtcccggccccattcctgaaaaaaggggagaatgtgattgatattctggacctgcacgaaaaggagcccaagcctgtccgcggcatgaaggaacgcaacaaggaacccggaaagataaataccaaaaacctggacaaccagtggtaa

SEQ ID NO: 16 - protein, 643 aa, Akkermansia muciniphila
MMKQYGFSWSAALMAVGVGALAWAGPEAVQNVQKPALSGGPPVVFGFGGEGNQEFMLNGKPFQIRGAEMHPQRIPREYWRHRIRTAKAMGLNTIAFYVFWNDHEQPDGSFDFKTGNRDLEGFLKLCQEEGMWVLFRPGPYACGEWDLGGLPHYLLKDPKAKLRTTEDAKFMKAQTRYLEAVARVAEPFLAKNGGPILMTQLENEYGSYQRKDRKYMEWLKAFWSRKGFGPFYTSDGAGEHFLKGVVLPGVAVGLDPGLNDGHWAVANKCNPGVPVFSSETYPGWLRHWGEGNWAPTPGVVNHVRWFMDKGRSFSLFVFHGGTNFGFSAGANNGGPGKYQPDLTSYDYGSPVDEQGRMNEYYAQMREIILKKLPPEAAVPEPPADIPAMEIPEFTPAVHAGLWENLPKPFRSKFPQPPYFEQWNQNQGIAVYRTAVPSGPPETLEFTNVNDYAQVYLDGELVGTLDRRLGQKSVKLPERRKPGTLEVLVEAMGHINFHISMESDRKGIYGPVKLGTRELKNWTVRALPLKADSIVRAPKGKGPSQKREGAHFRAVVNIEEPQDTFLDMSRYVKGYVWVNGINVGRYWNVGPQLRLYVPAPFLKKGENVIDILDLHEKEPKPVRGMKERNKEPGKINTKNLDNQW

SEQ ID NO: 17 - DNA, 2169 bp, Akkermansia muciniphila
  source 1..2169: mol_type = genomic DNA
  CDS 1..2169: product = AmGH20B; protein_id = 18; translation = MLLFRVFSIPVRFFIALLIYFSWGPLAGGVVTAPVPEFSSPALI PYPSKVVRGAGEAGFRSVHVKVGSDVPGRDDLMKEIKDIFRTSGIQGSLNSGGAAEGA MTWELFPDARMKGEGYDLSVGLGKATVRAGSFGGFFNALQTLRQLVFNREGEFAMPVV RISDKPAFVLRGIMLDVGRYYMSPALIKEVMRRLSRYKINTLHLHLTDDPAWRLEVKK YPALTDGAFHWKSRLPGRFYTQAQLKDLTDYCARLNIQVIPEIDMPGHSQPFARAMKT GMQTEKGVSILKDVVDEAVSLFPGRFFHMGSDEAHISMKDFIPRMAEHIRGKGKEVVV WSPGGPHDKDSVLMCWGENEAGARMDKNMKRIDSNGFYIDWADSQSGVYQVFFQQPCE VPQGDDKALGAIMPVWCDGNLSSERRVLEQYPFYPCALTFAERVWRGSATKRRDYMAQ LPPRGTDGWKEFREFEQRLAFHRDHFFQGVPFAYVKQADVAWSLVGPFDHRGKNDTSF EPERRIAPSYRDGDRILAWKKTPVYGAAVHVRHLFAMFNMHRNQYRTDHWPTLMSREV GKEDGTCYALTFIRSPREQEVWLMFGLNGMWGHSGGYRSARAPEQGSWDFSGGDVWLN GRRVNPPRWPFKSLPWTGWGKGRIEEAPLTWEGYFFRPPVKIKLRKGLNRVLIRSVFG HWKGDDGQRSWFFCCIPVLWDGIHYREVPGLEYDPRPDAR
atgcttttgttcagagtgttcagcattcccgtacggttttttattgccttgctgatttatttttcatgggggccgctggccggaggggtggttaccgcgcctgtgccggagttttcttcccccgcgcttattccatatccttccaaagtcgtcaggggcgcgggcgaggcaggtttcaggagcgttcatgtgaaggttggttcggatgttcctggcagggatgatttgatgaaggagatcaaggacatattcaggacttccggcattcagggttccctgaatagtgggggggctgctgaaggtgccatgacatgggaattgtttccggacgccaggatgaaaggagaggggtatgacctgtccgtcggattgggaaaagcaaccgtcagggcaggaagttttggcggtttttttaacgctcttcaaacgctgcggcaactggttttcaatagagaaggggaatttgccatgcctgttgtgcgtattagtgacaaacccgctttcgtcctgcggggaattatgctggatgtggggcgttattacatgtctcctgccttgatcaaggaggtgatgcgccgtctttcccgctataagattaatacgctgcatcttcatttgacggatgatcccgcatggaggctggaagtgaagaagtatccggccctgacggatggggcttttcattggaagtcccggctcccggggcggttttatacacaggctcaattgaaggatctgacggattattgcgcccggttgaatatccaggtgattccggagattgacatgccgggacacagccagccttttgccagggccatgaagaccggcatgcagacggagaagggtgtttccatattgaaggatgtcgtggacgaggccgtttccctgtttcccgggaggtttttccacatgggatccgacgaggcgcacatttccatgaaggattttatcccacgtatggcggagcatatccgcgggaagggcaaggaggtggtcgtgtggtctccgggcggcccgcacgataaggattccgtattgatgtgctggggagagaacgaggccggcgccaggatggataagaatatgaagcgcatcgacagcaatggtttttacattgactgggcggattcccagtccggcgtgtaccaggtgtttttccagcagccctgcgaggtgcctcaaggggatgataaggcgttgggggccatcatgcccgtgtggtgcgacggaaacctgagcagtgagaggcgggtgctggaacagtatccgttttatccttgtgcattgacgtttgcagagcgtgtctggcgcggaagcgctaccaagaggagggattatatggcccagcttccccccaggggaacggacggctggaaggaattccgggagtttgaacagcgtttggctttccatcgtgatcattttttccaaggcgttccttttgcctatgtgaaacaggcggacgttgcctggagcctggtggggccattcgaccaccgggggaagaatgatacctcttttgaaccggaaagaagaatagccccttcctacagggacggggacaggatactggcctggaagaagactcctgtttacggcgccgccgtacatgtcaggcatctttttgcgatgttcaacatgcaccggaaccagtaccggacggaccactggcccacgctgatgtcaagggaggttggaaaggaggatggaacctgctatgctctgactttcatccgcagccccagggagcaggaggtgtggctgatgtttggcctgaacggcatgtgggggcattcaggcgggtaccgcagcgcacgcgcccctgaacagggaagctgggatttttccggcggggacgtatggttgaacggaaggcgcgtgaaccctcccaggtggcctttcaagagcctgccctggacggggtggggaaaggggcgcattgaagaagctcctctgacctgggaagggtattttttccgccctcctgtaaagatcaagctccgcaaggggttgaaccgtgtattgatccgcagcgtgttcgggcactggaagggagacgacggccagagaagctggtttttctgctgcattcccgtcctgtgggacggcattcattaccgggaggttcccggcctggaatatgacccccgtccggatgcccgctga

SEQ ID NO: 18 - protein, 722 aa, Akkermansia muciniphila
MLLFRVFSIPVRFFIALLIYFSWGPLAGGVVTAPVPEFSSPALIPYPSKVVRGAGEAGFRSVHVKVGSDVPGRDDLMKEIKDIFRTSGIQGSLNSGGAAEGAMTWELFPDARMKGEGYDLSVGLGKATVRAGSFGGFFNALQTLRQLVFNREGEFAMPVVRISDKPAFVLRGIMLDVGRYYMSPALIKEVMRRLSRYKINTLHLHLTDDPAWRLEVKKYPALTDGAFHWKSRLPGRFYTQAQLKDLTDYCARLNIQVIPEIDMPGHSQPFARAMKTGMQTEKGVSILKDVVDEAVSLFPGRFFHMGSDEAHISMKDFIPRMAEHIRGKGKEVVVWSPGGPHDKDSVLMCWGENEAGARMDKNMKRIDSNGFYIDWADSQSGVYQVFFQQPCEVPQGDDKALGAIMPVWCDGNLSSERRVLEQYPFYPCALTFAERVWRGSATKRRDYMAQLPPRGTDGWKEFREFEQRLAFHRDHFFQGVPFAYVKQADVAWSLVGPFDHRGKNDTSFEPERRIAPSYRDGDRILAWKKTPVYGAAVHVRHLFAMFNMHRNQYRTDHWPTLMSREVGKEDGTCYALTFIRSPREQEVWLMFGLNGMWGHSGGYRSARAPEQGSWDFSGGDVWLNGRRVNPPRWPFKSLPWTGWGKGRIEEAPLTWEGYFFRPPVKIKLRKGLNRVLIRSVFGHWKGDDGQRSWFFCCIPVLWDGIHYREVPGLEYDPRPDAR

SEQ ID NO: 19 - DNA, 1557 bp, Akkermansia muciniphila
  source 1..1557: mol_type = genomic DNA
  CDS 1..1557: product = AmGH20D; protein_id = 20; translation = MKSILLAAAFLGSLCWAGTNPYNIIPEPVNVTTTSGTTKNLKIV HEQKVAGLGNEGYAMKLTPGGVELRYTTPNGKAMAMATLFQLQDQLSDTPEGLPCGSI QDSPDFGWRGMMVDVGRYHYPMKEIYNFVDAMHYYKYNVLHLHLTEDQGWRLPVPGYD KLRTIGAVRPSAPESQNNSLLANEGMYTKKELQDLVAYCKARGIQVLPEVEMPGHNMA LAASYPEFCCNTKRAQVWTHGGVSSKLICPQKPATKKFLKDTFNTVQQIFPFPYIHIG GDECPMGDWKKCPDCQAARAKKGQGDNVEAQMSDFTKSLTAMLAKHRKKPILWYDINK SYYHKGETVMSWLPGEFPRCIDKTKEQGIDLIVTPQFKYYLARTQMKFPADDVRARPG GAPILLKDCYNFDPRNGRDKNDVKHIKGINLCMWAEWIPSGELLMYMTYPRAMAVSET AWGSHKNRPSLEEFEKKMETHKKHFQKRFGYTLERTVENKPYREKFITQEEIERINEN YKKGQQNADK
atgaaatcaattttgctagccgcagcgttcctgggctccctctgctgggccggaaccaatccctacaacattattccggagcccgtcaacgtgacgacaacttccggaactaccaaaaacctcaaaatcgtccatgagcaaaaagtcgccggactgggcaatgaagggtatgccatgaaactaacgcccggcggcgtggaactccgttataccacgcccaacgggaaggccatggccatggcgaccctgttccagctccaggaccagctttcggatactcctgagggacttccctgcggcagcatccaggattcccccgacttcggctggcgcggcatgatggttgacgtaggccgctaccactatcccatgaaggagatctacaattttgtggacgccatgcattattacaaatacaacgtcctgcatctccatctgacggaagaccagggctggcgtctccccgttccgggctacgacaagctccgcaccatcggcgccgtccgcccctccgctccggaaagccagaacaactccctgctggccaatgaaggcatgtataccaagaaggagctccaggacctggtagcctactgcaaagcgcgcggcatccaggtactgccggaagtggaaatgccgggtcataacatggccctggccgcgtcctatcccgaattctgctgcaacaccaaacgggcccaggtatggacgcacggcggtgtttcctccaagctgatttgcccgcagaaaccggccactaaaaagtttcttaaggataccttcaataccgtccagcagatattccctttcccgtacatccacatcggaggtgacgaatgccccatgggggactggaagaagtgcccggactgccaggccgcccgagccaaaaagggccagggggataatgtggaagcccagatgagcgatttcacgaaaagcctgacggccatgctcgccaagcaccggaaaaagcccatcctgtggtatgacatcaacaagagctattaccacaagggggaaaccgtcatgtcctggctgccgggagaattcccgcgttgcattgataagacgaaggaacagggcatcgacctcatcgtcaccccccagttcaagtattatctggcgcgtacccagatgaaattcccggcggacgacgtgcgcgcccggcccggtggagctcccatcctgctgaaagactgctacaacttcgatccccgcaacggacgggacaagaatgacgtcaagcacatcaagggaatcaacctctgcatgtgggcggaatggattccctccggcgaattgctgatgtacatgacctacccccgcgccatggctgtttccgaaaccgcgtggggcagccacaagaaccgtccaagcctggaagagtttgaaaagaaaatggaaacccacaagaaacatttccagaagcgtttcggctatactctggaacgcactgtggaaaacaaaccctaccgggaaaaattcatcacccaagaggaaatcgaacgtattaacgagaattataaaaagggccagcaaaacgcggacaaatag

SEQ ID NO: 20 - protein, 518 aa, Akkermansia muciniphila
MKSILLAAAFLGSLCWAGTNPYNIIPEPVNVTTTSGTTKNLKIVHEQKVAGLGNEGYAMKLTPGGVELRYTTPNGKAMAMATLFQLQDQLSDTPEGLPCGSIQDSPDFGWRGMMVDVGRYHYPMKEIYNFVDAMHYYKYNVLHLHLTEDQGWRLPVPGYDKLRTIGAVRPSAPESQNNSLLANEGMYTKKELQDLVAYCKARGIQVLPEVEMPGHNMALAASYPEFCCNTKRAQVWTHGGVSSKLICPQKPATKKFLKDTFNTVQQIFPFPYIHIGGDECPMGDWKKCPDCQAARAKKGQGDNVEAQMSDFTKSLTAMLAKHRKKPILWYDINKSYYHKGETVMSWLPGEFPRCIDKTKEQGIDLIVTPQFKYYLARTQMKFPADDVRARPGGAPILLKDCYNFDPRNGRDKNDVKHIKGINLCMWAEWIPSGELLMYMTYPRAMAVSETAWGSHKNRPSLEEFEKKMETHKKHFQKRFGYTLERTVENKPYREKFITQEEIERINENYKKGQQNADK

SEQ ID NO: 21 - DNA, 1644 bp, Akkermansia muciniphila
  source 1..1644: mol_type = genomic DNA
  CDS 1..1644: product = AmGH20E; protein_id = 22; translation = MMFRHVSHVFLFLFSGLCGTAVNAQDQIIPRPVSVKVEAKGAAT PVKLGEGMRIICKEKDGEFRRQAHLLQQFLSRGTGLALDGAGGTGIIRVVKDAALKQY GPEAYRLEAAPGNIVISAATPKGVYYAGQSLAQMLPAAFFNNDADKKSVSWNVAEKPF SILDYPRFAWRAFMLDEARHFFGEEEVKRLIDQMGLLKMNVLHWHLSDDAGWRIQIKK YPKLTSVGGKRRDTEIETWGSGKYEGRPHEGFYTQEQVRRIVRYAADRNITIVPEIDI PGHSAAAIVSYPELKLSARPFAEVPVSFNDGAAFDPTSERTYQFLGDIMTELASLFPG GIIHIGGDEVRYKKYWEGVPHIEAFMKKKGIKTFPDLQIMFTNRISGMLAGMGRRMMG WNEILGSDVHNDGGRGAALGKLDANAIIHFWYGSDKIAAKAIREGRQVVNSTSHMTYI NKGYDKLPLSRSYSFEPVFAGLNPEQQKNVIGLGCQVWTEWIADVEKLHRHVFPRIAA YAETGWTRKEDKNFQDFQRRLTGYEKILDAQGIRHGEKE
atgatgttcagacatgtttcccatgtgtttctttttctgttttccggcttatgcggcacggccgtgaatgctcaggatcagataataccccggcccgtgtccgtgaaggtagaggccaagggggcggcaacgcccgtgaagctgggtgaaggaatgcgcattatatgcaaggagaaggacggggagttccggcgccaggctcacttgttgcagcagtttttgtcacgcggaaccggattggctctggacggggctggcggtacgggaataatccgcgttgtgaaggacgctgccctgaagcagtacgggccggaggcataccgtttggaagcggctcccggaaatatcgtcatcagcgccgcgactcccaaaggagtttattatgcggggcaaagtctggcgcagatgctgcctgccgcctttttcaacaatgatgcggacaaaaagagcgtaagttggaatgtagcggagaagcctttttccatattggattatccccgttttgcctggcgagccttcatgctggatgaagcacggcatttttttggagaggaggaggtgaagaggctgattgaccaaatggggctcctgaaaatgaatgtcctgcactggcatttgtcggatgatgccggatggcgcatccagattaaaaaatatcccaaactgacatccgtgggaggcaagaggagagatacggagatagagacgtggggaagcggcaagtatgaaggcaggccccatgaaggcttttatacgcaggaacaggtcaggcgcattgtccggtatgcggccgacaggaatataacgattgttcctgaaatagatattccgggccattccgccgccgccatcgtttcctatccggagttgaaattgtccgccaggcctttcgcggaagtgcccgtgagtttcaatgacggagcggcgtttgatcccaccagcgaacgtacctaccagttcctgggagacatcatgacagagctggcttccctctttcccggaggtatcattcacatcggcggtgacgaggtgcggtacaaaaagtactgggaaggcgtgccccacattgaggcgttcatgaagaaaaaaggcattaagacttttcccgaccttcaaatcatgtttacgaatcggatttccggcatgctggctgggatggggcgccggatgatggggtggaatgaaatactggggtccgacgtgcacaatgacggcggccggggagctgcgctcggcaagctggatgcaaatgctatcattcatttctggtatgggtccgataaaattgccgccaaggcgatcagggaaggccgccaggtggtgaattccacttcccacatgacttacattaacaaaggatatgacaagctccctctgtccaggtcctattcttttgagcccgtttttgcgggattgaacccggagcagcagaagaatgtcatcgggctgggctgccaggtctggacggaatggattgcggacgtggagaaactgcaccgtcatgtatttccccgtattgccgcttatgcggagacggggtggacccggaaggaggataagaattttcaggatttccagaggcgtttgacggggtatgaaaaaattttggatgcccagggcatccggcacggtgaaaaggagtag

SEQ ID NO: 22 - protein, 547 aa, Akkermansia muciniphila
MMFRHVSHVFLFLFSGLCGTAVNAQDQIIPRPVSVKVEAKGAATPVKLGEGMRIICKEKDGEFRRQAHLLQQFLSRGTGLALDGAGGTGIIRVVKDAALKQYGPEAYRLEAAPGNIVISAATPKGVYYAGQSLAQMLPAAFFNNDADKKSVSWNVAEKPFSILDYPRFAWRAFMLDEARHFFGEEEVKRLIDQMGLLKMNVLHWHLSDDAGWRIQIKKYPKLTSVGGKRRDTEIETWGSGKYEGRPHEGFYTQEQVRRIVRYAADRNITIVPEIDIPGHSAAAIVSYPELKLSARPFAEVPVSFNDGAAFDPTSERTYQFLGDIMTELASLFPGGIIHIGGDEVRYKKYWEGVPHIEAFMKKKGIKTFPDLQIMFTNRISGMLAGMGRRMMGWNEILGSDVHNDGGRGAALGKLDANAIIHFWYGSDKIAAKAIREGRQVVNSTSHMTYINKGYDKLPLSRSYSFEPVFAGLNPEQQKNVIGLGCQVWTEWIADVEKLHRHVFPRIAAYAETGWTRKEDKNFQDFQRRLTGYEKILDAQGIRHGEKE

SEQ ID NO: 23 - DNA, 2187 bp, Akkermansia muciniphila
  source 1..2187: mol_type = genomic DNA
  CDS 1..2187: product = AmGH20F; protein_id = 24; translation = MNRMKFLLLSFAWVCMACAGAWGQDTAPSFPANGANYRLFPADR PPLVPKPQQLRWDDRAIPVQSVRILAPSPSRTSYPEQMKFIVSELKSFLAEHCVKVAP DGTFAVKFVKGDVKAGTENSKLKEEAYSLRVTSGGALITAMDTRGFYYGMKTLEQLLL RRGGTTTIAACDIVDWPDFEIRGFMNDVGRNYMPLPLIARELDSMAQLKLNVYHFHFT ENPGWRLESKIYPELNAPENYTRMPGKFYTQKEFKQLVEYCRLRNILLIPEMDMPGHS QMFRKALNVKMSDEKATKALVALIKELCSLVPKEKMPIIHIGTDEVRGKDEQVNNEIL KEYIHAVEFCGRIPMRWQPGLTPKGYNGSIQQLWSGRQNRGAWPTDGAKYVDSLETYL NHLDPFETAMTMYFRRACPFRNAEGLGMMLCSFPDLEITDPRNQVLQTPVYAGMAFVS EPLWNNPHEKVLGDPNQDEYMKYFSNLPVQGDPLLKGFADYENRVLAIRDRFFVDKEF NYVRQANIPWKLLGPIPNGGKTEKEFAPEEDNKAGKMRDSYEIDGVTYEWSGDDYTGA TIIFKHYCDFPTLFNGAKMGAYPHKNHTYYAQTWIYSPKAQTVPFWISGHTWATSDWR NGPASVPGKWFHADPKFFVNGREIAPPQWKKPRNSGVMVDENYHFREPSMVPLKKGWN RVLVKSPSNNSARRWMFTFVPVLVNPKTPGCNVKEYPGLKFSTRPE
atgaaccgcatgaagttccttttattatcgtttgcatgggtgtgcatggcttgcgccggagcatgggggcaggatacggccccgtctttcccggctaacggggccaattacaggctgtttccggcggaccggcctccgctggttcccaaaccccagcagctgcgctgggacgacagggccattcccgtgcagtccgtacgcattttggctccgtctccgtccaggacttcctatccggaacagatgaagttcattgtttccgaattgaaatcttttctggcggagcactgcgtgaaggtggctccggacgggacgtttgccgttaaattcgtcaagggggatgtgaaagccggcacggaaaattccaagctgaaggaggaggcttattccctccgagtaacttccggcggcgcactcattacggcgatggataccagaggattctattacggcatgaaaacgctggagcagcttcttttgcgccgcggcgggacgacgaccattgccgcctgcgatatcgtggactggccggattttgaaatccgcggattcatgaacgatgtgggacgcaattacatgccgctgcctctgattgcacgggagctggattccatggcgcagctcaagctgaatgtttaccacttccattttacggagaaccccggctggcggctggaatccaaaatttatccggagctgaacgcccccgaaaattatacgcgcatgcctggcaagttttacacgcagaaggagtttaagcagctggtggagtactgccgcctgcgcaatatcctgctgattccggagatggatatgccggggcacagccagatgttccgcaaggcgctcaacgtgaagatgagcgatgaaaaagccaccaaagccctggtggccctgatcaaggagttgtgttccctggttcccaaggagaaaatgcccatcatccacattggcacggacgaggtgcgcggcaaggatgagcaggtgaacaatgagattcttaaggagtacatccatgcagtggagttctgcggccgcattcccatgagatggcagcccggcctgacgccgaagggctataacggctccatccagcagttatggtccggccgccagaaccgtggcgcatggcctaccgacggagcgaagtatgttgattccctggagacttacctgaaccaccttgatccgtttgaaacggccatgaccatgtatttccgccgggcatgcccgtttcggaatgcggaaggactgggcatgatgctgtgttctttcccggacctggaaattacggatccgcgcaaccaagttcttcagacgcccgtttacgccggcatggcgttcgtttccgaacctttgtggaataatccccatgagaaggtgctgggagaccccaaccaggacgaatatatgaagtatttttccaatctgcccgtgcagggggatcctctgctgaaggggtttgcggattacgagaaccgcgtgctcgccatccgggaccgttttttcgtggataaggagtttaattacgtacggcaggcgaatattccctggaaattgctggggcctattcccaacggcggtaagacggaaaaggaattcgctccggaggaggacaacaaggcagggaagatgagggattcctacgagattgacggcgtcacctatgagtggtccggagacgattacacgggggccaccatcattttcaagcattactgcgattttccgacgctgttcaatggcgcaaagatgggagcttatccccacaaaaaccacacttattacgcgcagacctggatttattcccccaaggcgcagacggtgcctttctggatcagcggacatacctgggccacgtccgattggcgcaacggtccggcgagcgttcccggcaagtggtttcatgcggatcccaaattttttgtgaacggccgggagattgcccccccgcaatggaaaaagccgcgtaacagcggcgtgatggtggatgaaaactaccatttccgggagccttccatggttcctcttaagaagggttggaaccgcgtgctggtaaagagccccagcaacaattccgcgcgtcggtggatgttcacattcgttccggtgctggtgaaccccaagacgcccggctgcaatgtgaaggagtatcccggcctcaaattttccacacgtccggaatag

SEQ ID NO: 24 - protein, 728 aa, Akkermansia muciniphila
MNRMKFLLLSFAWVCMACAGAWGQDTAPSFPANGANYRLFPADRPPLVPKPQQLRWDDRAIPVQSVRILAPSPSRTSYPEQMKFIVSELKSFLAEHCVKVAPDGTFAVKFVKGDVKAGTENSKLKEEAYSLRVTSGGALITAMDTRGFYYGMKTLEQLLLRRGGTTTIAACDIVDWPDFEIRGFMNDVGRNYMPLPLIARELDSMAQLKLNVYHFHFTENPGWRLESKIYPELNAPENYTRMPGKFYTQKEFKQLVEYCRLRNILLIPEMDMPGHSQMFRKALNVKMSDEKATKALVALIKELCSLVPKEKMPIIHIGTDEVRGKDEQVNNEILKEYIHAVEFCGRIPMRWQPGLTPKGYNGSIQQLWSGRQNRGAWPTDGAKYVDSLETYLNHLDPFETAMTMYFRRACPFRNAEGLGMMLCSFPDLEITDPRNQVLQTPVYAGMAFVSEPLWNNPHEKVLGDPNQDEYMKYFSNLPVQGDPLLKGFADYENRVLAIRDRFFVDKEFNYVRQANIPWKLLGPIPNGGKTEKEFAPEEDNKAGKMRDSYEIDGVTYEWSGDDYTGATIIFKHYCDFPTLFNGAKMGAYPHKNHTYYAQTWIYSPKAQTVPFWISGHTWATSDWRNGPASVPGKWFHADPKFFVNGREIAPPQWKKPRNSGVMVDENYHFREPSMVPLKKGWNRVLVKSPSNNSARRWMFTFVPVLVNPKTPGCNVKEYPGLKFSTRPE

SEQ ID NO: 25 - DNA, 1995 bp, Akkermansia muciniphila
  source 1..1995: mol_type = genomic DNA
  CDS 1..1995: product = AmGH20G; protein_id = 26; translation = MRLSLSAPLLASGLMLCFTSSHGDWTSPHPLQQEKAAPVPLIPF PSQVDWKTGTCPKKAPVSVKKNASLSKTLGKEGYELRIRPNGILIKAADDAGVFYARR TLDQLGARGDYPCCDIKDSPAFAIRCFMHDAGRHFRTVETLKADIDEMARLKINAFHW HLTDYPAWRIQCKKYPVLNDPSKRIQGRDVNGTYSYDQIRDLFRYARERHVQIIPEID MPGHSTYFKNCFGFPMHDPRGINILEELLEEFCREIPAEMSPYLHIGADEIRIPNGKQ FADRMAAKVKSLGRQPIQWAGNNDLPVSGDSYAQLWNDENSVGLPDPARQKNPYFDST AGYVNSFDPGILVRRNFFRQPCGTARGNNHSLGVIQCLWPDTRVENKKNIPVQSPQWP AMFAMAERSWKGIPEDGSRFAGSLPEKNTEAYQAFSLFEKRMEALAGSRPFPYWRDSF VEWTVFGPVPQDRQEEVRNNLLAGKSPAGLSSVQTRGGNLYFRTRAGAEGLFSKTKPG NTVWAETTFHAPVEGTMHAMVGFDAPARSTRRCSGVPAAGEWSQCGTRIWVNGKEMKN PQTYKLAGQRRYEKHTWNSPANEIPFDNEEFWWARPPVPFQVKAGENRILIEQPYTGE FQSWGVSFIPVKKAGDRWIADPSYYAKPRREKQDDVSPVP
atgcgattatccctttccgcgcctttgctggcttccggcctgatgctttgttttacctcctcccatggagactggacatccccgcaccctctccagcaggaaaaagccgctccggtcccactgattcccttcccgtcccaggtggactggaaaacgggaacatgccctaaaaaggctcccgtttccgtgaaaaaaaacgcgtccctgtctaagacgctgggcaaggaagggtatgaactgcggatccgccccaacggcattctgattaaggccgctgacgatgcgggcgttttttatgcccgcagaacgctggaccagctgggagccaggggagattacccgtgctgcgacatcaaggacagccccgcgttcgccatccggtgcttcatgcatgacgcaggccgccatttccggactgttgaaacactcaaggcggatattgatgaaatggcccggctgaaaataaacgcttttcactggcatctgacggattaccccgcatggcgcatccagtgcaaaaagtaccctgttttgaatgatccctccaagaggattcagggacgggacgttaacggcacctactcctacgaccagatacgggacttattccgctacgccagggagcgccatgtccagattattccggaaatcgatatgcccgggcacagcacctattttaaaaactgctttggcttcccgatgcatgatcccagaggcatcaacattctggaagaactgctggaagaattttgcagggaaattccggcggaaatgtctccctaccttcatatcggagcggatgaaatccgcattcccaacggaaagcagtttgcggaccggatggcggccaaggtcaaatccctgggacggcagcccatccaatgggcgggcaacaatgacctgcctgtgtccggagacagttatgcccagctgtggaatgatgaaaattccgtaggcctgccggatccagccaggcagaagaatccctacttcgactccacggcaggatacgtcaattcctttgaccccggcattctggtgcgcaggaatttcttccgccagccctgcggaacagccaggggaaacaaccattccctgggcgtcatccagtgtctatggccggatacccgggtggagaacaaaaaaaacattcctgtccagagcccccagtggcctgccatgttcgccatggcggaacgcagttggaaaggaattccagaggatggttcccgttttgccggcagtctgccggaaaagaacacggaggcttatcaggccttctctctgtttgaaaaacgcatggaagccctggccggaagcaggcctttcccttactggagggattctttcgtggaatggacggtattcggccccgttccgcaggacaggcaggaagaagtaagaaataacctgctggcgggcaaatctcccgcaggactgtcttccgtccagacgcgaggaggcaatttgtatttccgtacccgtgcgggtgcggagggtctattttccaagacaaaaccggggaatacggtctgggcggagacgacgtttcacgcgcccgtggagggcaccatgcacgctatggtggggtttgacgctccggcccgttccacacggcgctgttccggagtaccggctgccggggagtggtcccagtgcggcaccagaatatgggtaaacggcaaggaaatgaaaaatccccagacttacaaactggcaggccaacggcgttacgaaaagcatacatggaattcgcccgctaatgagataccctttgacaacgaggaattctggtgggcccggcctcctgttccctttcaagtgaaggccggggaaaacaggatcctgatagaacaaccgtacacaggagaattccagtcgtggggagtcagcttcattccggtgaaaaaagcgggagaccgctggattgccgacccaagctattatgccaaacccaggagagagaaacaggatgacgtctctccggtgccataa

SEQ ID NO: 26 - protein, 664 aa, Akkermansia muciniphila
MRLSLSAPLLASGLMLCFTSSHGDWTSPHPLQQEKAAPVPLIPFPSQVDWKTGTCPKKAPVSVKKNASLSKTLGKEGYELRIRPNGILIKAADDAGVFYARRTLDQLGARGDYPCCDIKDSPAFAIRCFMHDAGRHFRTVETLKADIDEMARLKINAFHWHLTDYPAWRIQCKKYPVLNDPSKRIQGRDVNGTYSYDQIRDLFRYARERHVQIIPEIDMPGHSTYFKNCFGFPMHDPRGINILEELLEEFCREIPAEMSPYLHIGADEIRIPNGKQFADRMAAKVKSLGRQPIQWAGNNDLPVSGDSYAQLWNDENSVGLPDPARQKNPYFDSTAGYVNSFDPGILVRRNFFRQPCGTARGNNHSLGVIQCLWPDTRVENKKNIPVQSPQWPAMFAMAERSWKGIPEDGSRFAGSLPEKNTEAYQAFSLFEKRMEALAGSRPFPYWRDSFVEWTVFGPVPQDRQEEVRNNLLAGKSPAGLSSVQTRGGNLYFRTRAGAEGLFSKTKPGNTVWAETTFHAPVEGTMHAMVGFDAPARSTRRCSGVPAAGEWSQCGTRIWVNGKEMKNPQTYKLAGQRRYEKHTWNSPANEIPFDNEEFWWARPPVPFQVKAGENRILIEQPYTGEFQSWGVSFIPVKKAGDRWIADPSYYAKPRREKQDDVSPVP

SEQ ID NO: 27 - DNA, 1482 bp, Akkermansia muciniphila
  source 1..1482: mol_type = genomic DNA
  CDS 1..1482: product = AmGH20H; protein_id = 28; translation = MARPLPILGGILLSFSPPAEATAQYSIIPEPSRTELRQETAKTL QLLSDQEVPTLETDAYRLTVTPQGAHLASGGREGRIYGLATLRQLRDQLAGQPEGIPC GVITDKPRYPWRGLMVDPARHFIPAADLKKFVDMMAYYKFNRLHLHLTDNQGWRLPVP GYPKLKSVASRREESFGDGIPHEGMYTKQELKELVAYCAARGIDVIPEIDMPGHNQAL HAAYPEFFCFPKPDMNVRTTAGNSKELVCPQKPEVWKFYASVFNELKDIFPSGIVHLG GDEAPTELWEKCPLCREARTRAAMKDEQEQMKAFFAKTAALLAKNGQTPQFWYEGNAG IYHPGETVYAWRQGQALQSIEKTKKAGLNLIMASSEYCYLDFPQIQGQRNWGWMKTTT LQKCYDLDPAFGKPEKEAGHIRGVHAPVWAERLPDLNHLLYRAYPRACAIAEAGWSPM GVRSWENFRRKLADHRQFILKRFNYDMERTQGNEPAFRWENNK
atggcacgtcccttacccattctcggcggcattctgctatccttctcccctccggcagaagcaacagcccaatacagcattatccctgagccgtccagaacggaactcagacaggaaacagctaaaactttacagcttctttccgaccaggaagttccgaccctggaaacggacgcctaccggctcacggtcaccccgcagggggcgcaccttgcttccggaggaagggaaggcagaatttacgggctggcaaccctccgccagctccgggaccagctggcgggacagccggagggcattccctgcggcgtcatcacggacaagccgcgctatccgtggcgcggcctcatggtggatcccgcgcggcatttcatccccgcggccgatctgaaaaaatttgtggatatgatggcctactacaaattcaacaggctgcacctgcatctgacggacaaccagggctggaggctgcccgtgcccggctaccccaaattgaaaagcgtcgcatccaggcgggaggaaagcttcggagacggaatcccccacgaagggatgtacaccaaacaggaactgaaggaactggtggcgtactgcgcagcgcgcggcattgatgtcatccctgaaatagacatgccgggccacaaccaggcgcttcatgccgcctacccggaatttttctgcttccccaaaccggacatgaacgtgcggacgacagcgggaaacagcaaggaactggtctgtccccagaagccggaagtctggaaattttatgcctccgtctttaatgaactcaaggatatcttcccgtccggtatcgttcatctgggcggggacgaggcccccacggaactctgggaaaaatgccctctgtgccgggaagcccggaccagggcagccatgaaagacgaacaggaacagatgaaagccttttttgcgaaaacggcagctctgcttgccaaaaacgggcaaacgccgcaattctggtatgaggggaacgccggcatttaccatccgggggaaacggtttacgcatggcggcaaggccaggccctccagtccattgagaagacgaaaaaggcgggattgaacctgattatggcctccagcgaatactgttacctggattttccccagattcaggggcagcgcaactggggatggatgaaaaccaccaccctgcaaaaatgttatgacctggatcccgcttttggaaaaccggagaaagaggcaggccatatccggggcgtgcatgcccccgtatgggcggaacgcctgccggacttgaaccacttgctttaccgcgcctatccccgcgcctgcgccattgcggaagccggctggtcaccgatgggcgtgcgctcctgggaaaacttccggcgcaagctggccgaccaccgtcaattcatcctcaaacgcttcaattatgatatggagcgcactcaggggaatgaaccggccttccgctgggaaaacaacaagtaa

SEQ ID NO: 28 - protein, 493 aa, Akkermansia muciniphila
MARPLPILGGILLSFSPPAEATAQYSIIPEPSRTELRQETAKTLQLLSDQEVPTLETDAYRLTVTPQGAHLASGGREGRIYGLATLRQLRDQLAGQPEGIPCGVITDKPRYPWRGLMVDPARHFIPAADLKKFVDMMAYYKFNRLHLHLTDNQGWRLPVPGYPKLKSVASRREESFGDGIPHEGMYTKQELKELVAYCAARGIDVIPEIDMPGHNQALHAAYPEFFCFPKPDMNVRTTAGNSKELVCPQKPEVWKFYASVFNELKDIFPSGIVHLGGDEAPTELWEKCPLCREARTRAAMKDEQEQMKAFFAKTAALLAKNGQTPQFWYEGNAGIYHPGETVYAWRQGQALQSIEKTKKAGLNLIMASSEYCYLDFPQIQGQRNWGWMKTTTLQKCYDLDPAFGKPEKEAGHIRGVHAPVWAERLPDLNHLLYRAYPRACAIAEAGWSPMGVRSWENFRRKLADHRQFILKRFNYDMERTQGNEPAFRWENNK

SEQ ID NO: 29 - DNA, 2271 bp, Akkermansia muciniphila
  source 1..2271: mol_type = genomic DNA
  CDS 1..2271: product = AmGH20J; protein_id = 30; translation = MKKNLFLMIAVLAASPVMGQDAKQIADSLSIPPVKAGAKQLPMP SVSGAQIKLLGADYEQLVNSKGKIAPVISDTPVNVSFKVTKDGKEAVSKDYEIMLQAP QAAQGNPKPRIIPEILQWKGGQGEYKLGNTVTIACPDKELGKLFAADMEDVLGKKVKL VAPGAKADISLSLLKGGNLGREGYRLQIARDGVRLGAAAPTGLFWGTRTLLQMLRQTP GSVPCGTAVDFPRYQLRGFMLDVARTPYPLSYLKDVIRTMAWYKMNDLHLVINNNYIF HEHYVDNGHDPFKESYAAFRLESKMKGKDGTPLTARDLFYTKKEFADLVSYARKYGVN IVPEFDTPGHALSFTRLRPDLIYKGPMNHEKRRCEMLDAANPETIDLVSKVFDEYMLK DPKLGRPVFADCGVVHVGADEFYGDKEDYRHFANAVLTHALKRGYTPRIWGSLSAKPG KTPVVSKGVQMNLWSTGWMKAWEAVNQGYDVINTNDGALYIVPFAGYYRMDRNHKGLY NNWIPNRIGNETLPSGHPQLLGGTFAVWNDETDIMHTGYAPYDIWGIISGSMDVLSQK LWGTAKAPDTFEQHRELVSSIGNAPRTNPLHKWKDSQPLTVKPSSLPQKLDKPALGPN YRLTMELELTAAPEGKEQVLLAAPEGELLAVMKDGTVGFRRDDSLEFSFGAKLPVGKK VKVEIVGEPEKTSLLLDGEPAGTAVLKNFSDKSKDFSDKFKHRPKVHRSTFILPLKEL GSSFQGKVFHMNVQPL
atgaagaaaaacctattcctgatgatcgccgttcttgccgcgtctcccgtgatggggcaggacgccaagcagattgcggattccctttccatccctccggtgaaagccggagcgaagcaactgcccatgccgagcgtttccggagcccagattaaattgctgggggccgactatgagcagctcgtcaacagcaaaggcaagatagcccctgttatttcagatacccccgtcaacgtcagcttcaaggtgaccaaggacggcaaggaagccgtcagcaaagattatgaaatcatgctgcaggctcctcaggccgcccagggcaaccccaagccgcggattatcccggaaatcctgcaatggaaggggggacagggagaatataagctggggaatacggtcaccatagcatgcccggacaaggagctgggcaaattgtttgcggcggatatggaagacgtgctggggaagaaggtcaagcttgtcgcccccggggcgaaggcggatatttccctttccctgttgaagggcggcaatctgggcagggaaggataccggctccagattgccagggacggcgtccgccttggcgcggcggctcccaccggcctgttctggggcacgcgcaccctgctgcagatgctccgccagacgcccggcagcgtgccctgcggcacggccgtggattttccgcgctaccagctccgcgggttcatgcttgatgtcgcgcgcacgccttaccccctcagttacctgaaggacgtgatccgcaccatggcctggtacaagatgaatgacctgcacctggtcatcaacaacaactatattttccacgaacactatgtggacaacggccatgatccgttcaaggaaagctatgcggctttccgcctggaatccaaaatgaagggaaaggacggaacgcccctgacagccagggatcttttttataccaagaaggaatttgccgacctggtcagctatgccagaaagtacggcgtcaatattgttccggagttcgacacgccggggcatgccctttcctttacgcgcctgcggcctgatctcatttataaagggcccatgaatcatgagaagcgccgctgcgagatgctggacgccgccaacccggaaacgatcgacctggtgagcaaggtttttgacgaatacatgctgaaggatcccaaactgggtcgtcccgtgttcgcggactgcggcgtggtgcatgtgggggcggatgaattttatggggacaaggaagattaccgccactttgcaaacgccgtgctcacccatgccctcaagcgcggttatacgccccgcatctggggaagcctcagcgccaagccgggcaagaccccggtggtcagcaaaggggtccagatgaacctttggagcaccggctggatgaaagcatgggaagccgtcaaccagggctatgatgtgatcaatacgaacgatggagccctttatatcgttccctttgccggctactaccggatggacagaaaccataaggggctgtacaataactggattcccaaccgcatcggcaatgaaacgctgccttccgggcatccccagctcctcggaggcaccttcgccgtctggaatgatgaaacggacattatgcatacggggtacgcgccttatgacatttgggggatcatttccggttccatggacgtgctcagccagaaactgtggggaaccgccaaagctccggatacgtttgaacagcatcgcgagctggtttcctccatcggcaacgctccgcgcaccaatcccctgcacaaatggaaagacagccagcccttgacggtgaagccttcctccctcccccagaagctggacaaacctgccctggggccgaattaccgcctgaccatggaactggaattgactgccgctccggaagggaaggagcaggtgctgcttgctgcgcctgagggcgaattgcttgcggtcatgaaggacggcaccgtcggattccgccgtgacgattctctggaattttccttcggcgccaagcttcccgtaggcaagaaggtcaaggtggaaattgtgggagaaccggagaaaaccagcctgttgcttgacggggaacctgccggaactgccgtgctgaagaatttttccgacaagtccaaggatttttcggacaaattcaagcatcgtcccaaagttcaccgttccacgttcattctgcctctgaaggaacttggctcctccttccaggggaaggtgttccatatgaacgtacagccgttgtaa

SEQ ID NO: 30 - protein, 756 aa, Akkermansia muciniphila
MKKNLFLMIAVLAASPVMGQDAKQIADSLSIPPVKAGAKQLPMPSVSGAQIKLLGADYEQLVNSKGKIAPVISDTPVNVSFKVTKDGKEAVSKDYEIMLQAPQAAQGNPKPRIIPEILQWKGGQGEYKLGNTVTIACPDKELGKLFAADMEDVLGKKVKLVAPGAKADISLSLLKGGNLGREGYRLQIARDGVRLGAAAPTGLFWGTRTLLQMLRQTPGSVPCGTAVDFPRYQLRGFMLDVARTPYPLSYLKDVIRTMAWYKMNDLHLVINNNYIFHEHYVDNGHDPFKESYAAFRLESKMKGKDGTPLTARDLFYTKKEFADLVSYARKYGVNIVPEFDTPGHALSFTRLRPDLIYKGPMNHEKRRCEMLDAANPETIDLVSKVFDEYMLKDPKLGRPVFADCGVVHVGADEFYGDKEDYRHFANAVLTHALKRGYTPRIWGSLSAKPGKTPVVSKGVQMNLWSTGWMKAWEAVNQGYDVINTNDGALYIVPFAGYYRMDRNHKGLYNNWIPNRIGNETLPSGHPQLLGGTFAVWNDETDIMHTGYAPYDIWGIISGSMDVLSQKLWGTAKAPDTFEQHRELVSSIGNAPRTNPLHKWKDSQPLTVKPSSLPQKLDKPALGPNYRLTMELELTAAPEGKEQVLLAAPEGELLAVMKDGTVGFRRDDSLEFSFGAKLPVGKKVKVEIVGEPEKTSLLLDGEPAGTAVLKNFSDKSKDFSDKFKHRPKVHRSTFILPLKELGSSFQGKVFHMNVQPL

SEQ ID NO: 31 - DNA, 1650 bp, Akkermansia muciniphila
  source 1..1650: mol_type = genomic DNA
  CDS 1..1650: product = AmGH20K; protein_id = 32; translation = MMNKGCCFRLAAWLALSGLPGAGMAEMLPAEEVLLPAPSACRSD RAAVAHLPLRLAVYAPEKDVPVVEALLNVLNAQGVLSGLSITRDKASADAVCSVEGTP SGTGEGYEARLKRDVEGRGILHLKAAAPQGLFYAWQSMVQILSANRTGEGFSVPVFSL RDDPRFEWRGLMLDVSRHFFTMAEVKRMVDTMSLFKLNRLHLHLTDGPGWRLEIKKYP LLTSMSAWRVPLADGEWNWQEVKLAIQEHDPEATYGGFYTQEQMKEIIDYARSRQVTV VPEIDLPGHSYAAMHAYGDLICDGVDFPVEGKKGRDAVCMGRPETLRFVKDVVDELKA VFPPGSPIHLGHDEVSAEAWKKCRYCQNRLKNLREKDLKALGRDFLQQIADYVRKDGY DAIVWDEAGELEADKHILVMAWRGNGFAAAAARKGHPVILAPCSHFYFDYYQSAASTE PKAVGGLIPLKQVYGYELPELSPEEAARVRGLQANIWTEYLYNMGLVEYMAWPRALAL SERAWSDCDPRDYKSFRSRASRALKILEKLAVKYRPMDEED
atgatgaataaggggtgttgtttccgtctggcagcatggctggccctgtccggattaccgggcgccggcatggcggaaatgctgccggccgaggaggtgctgctccccgcgccttccgcatgcaggtctgaccgggccgccgtggcgcatctcccgttgcggctggcggtctatgcgccggaaaaggatgttcctgtagtggaagctctcctgaatgtgctcaatgcccagggcgttttgtccggcctctccataacccgtgacaaagcctctgcggatgccgtctgttcggttgagggaaccccctcggggacaggcgaaggatatgaggcccgcctgaagcgtgacgtggaagggaggggcatcctgcatctgaaagcggcggcgccccagggccttttctacgcgtggcagagcatggtccagattttaagcgccaaccggaccggggaggggttttccgtacccgttttttccctccgggatgatccccgctttgaatggcggggcctgatgctggatgtttcccgccacttctttaccatggcggaagtcaagcgcatggttgacaccatgtccctgttcaagcttaaccgcctccacctgcacctgacggacggtcccggctggcgcctggaaattaaaaaatatccattgcttacctccatgagcgcctggcgcgttccgctcgctgacggagaatggaactggcaggaagtgaaactcgccatccaggagcatgacccggaagctacctatggcggtttttacacccaggagcagatgaaggagattattgactatgcgcgcagccgccaggttaccgttgttccggaaatagaccttcccggccattcctatgcggccatgcatgcctacggggatttgatttgcgacggtgtggattttcccgtggaggggaaaaaaggacgggatgccgtctgcatggggcgcccggaaacgctccgcttcgtgaaagacgtagtggatgaactcaaggccgtttttccgcccggctcccctatccatttgggccatgacgaggtgtctgccgaggcatggaaaaaatgccggtattgccagaaccgtttaaagaacctgagggagaaggatctcaaggccctgggccgggatttcctgcagcagattgcggactacgtgcggaaggacggttatgacgccatcgtctgggatgaagccggggaactggaggcggacaaacacatcctcgttatggcgtggcgcggcaacggctttgctgcggcggcagccaggaaagggcatcccgtcattctggcgccgtgttcccacttctattttgactattaccagtctgccgcatccacggagccgaaagccgtcgggggccttattccgctcaagcaggtgtacggctatgaattgccggaactctccccggaggaagccgccagggtgcgcggccttcaggctaacatctggacggaatacctgtacaatatggggctggtggaatacatggcatggccgcgcgccctggccctgtccgaacgcgcctggagcgactgcgacccccgggactacaaatccttccgcagccgtgcctccagggccctgaaaatcctggaaaaactggccgtgaaataccgccccatggatgaagaagactag

SEQ ID NO: 32 - protein, 549 aa, Akkermansia muciniphila
MMNKGCCFRLAAWLALSGLPGAGMAEMLPAEEVLLPAPSACRSDRAAVAHLPLRLAVYAPEKDVPVVEALLNVLNAQGVLSGLSITRDKASADAVCSVEGTPSGTGEGYEARLKRDVEGRGILHLKAAAPQGLFYAWQSMVQILSANRTGEGFSVPVFSLRDDPRFEWRGLMLDVSRHFFTMAEVKRMVDTMSLFKLNRLHLHLTDGPGWRLEIKKYPLLTSMSAWRVPLADGEWNWQEVKLAIQEHDPEATYGGFYTQEQMKEIIDYARSRQVTVVPEIDLPGHSYAAMHAYGDLICDGVDFPVEGKKGRDAVCMGRPETLRFVKDVVDELKAVFPPGSPIHLGHDEVSAEAWKKCRYCQNRLKNLREKDLKALGRDFLQQIADYVRKDGYDAIVWDEAGELEADKHILVMAWRGNGFAAAAARKGHPVILAPCSHFYFDYYQSAASTEPKAVGGLIPLKQVYGYELPELSPEEAARVRGLQANIWTEYLYNMGLVEYMAWPRALALSERAWSDCDPRDYKSFRSRASRALKILEKLAVKYRPMDEED

SEQ ID NO: 33 - DNA, 1791 bp, Akkermansia muciniphila
  source 1..1791: mol_type = genomic DNA
  CDS 1..1791: product = AmGH110B; protein_id = 34; translation = MQNPVASLLFILAMLTGPCPAADYPERTERTQSAGNHVWHIDPD KGNDGNPGTAPSTAWKSMAPANRLIMARGDTLVIHPGEHAVSLALMGEGSKQAPVTIR FMPGRHIFKHGALMTGKPQISNTNDAPNEPKAMAIRLMEAKNIRLEGKPGATDILLEG KAIFVCMEHAENVSLNGLGFDYLHPTMGEFLVTEVEGDTMKATIPDGTLYTVKDGNLT WHGPGWEFRMGGYSKVFDSASGTFQGRFDPGKTVIRELSPGKISITFKEGSPTMKPGQ SYQNRNTRRDCCGFFQYRSKNILWNNCHIYYMHGMGVVSQFCENIMFSHLKIAPRPRS LRTNSSWADNLHFSGCRGKIIVKDCVLGASHDDAVNVHGTHLRIIDRPAPNKITVRFM HPQTFGFDAFAAGDRIDYVSCNTLVPYASNTVSGVKQLNEKEIELTLQHPNPGNIQPD DVVENVTWTPSVHISNTVCRHIPTRGFLLTTRKPVLVERCRFEKTGMPAILVEDDASG WYESGVVRNMTISRNTFIQCGEAVIQIVPHAPRPEGDVHRNITITGNTFDLKNGTAIR IRHTGDVKAEKNTFTKDGKKIPEEKAVDIR
atgcaaaaccccgtggcctccctgctttttattctcgcaatgttgacagggccatgcccggcagcggattatccggaacggacggagcgcacgcaatccgcagggaaccatgtctggcatatagacccggacaagggaaatgacggcaatccgggaacggccccatccacggcgtggaaaagcatggcgccggccaaccgtctcatcatggcgcggggagatacgctcgtcatccatccgggggaacatgccgtctccctggcgctgatgggggaaggttccaaacaggccccggtcaccatccggttcatgcccggcaggcatatcttcaaacacggtgccctgatgaccggaaaaccgcagatttccaacaccaacgatgcgcccaacgagcccaaggccatggctatccgcctgatggaagccaaaaacatacgcctggagggaaagccgggagccaccgacatcctgctggaaggaaaagccatctttgtctgcatggaacatgcggaaaacgtttccctgaacggcctgggttttgattacctgcatccgaccatgggagaatttctggtcacggaagttgaaggggataccatgaaagcaactattccggacggaaccctgtacacagtgaaggatggaaacctgacctggcacggacccggctgggaattccgcatgggagggtattccaaagttttcgattccgcctccggcactttccagggccgtttcgaccccgggaaaacagtcatcagagaattgtctcccggaaaaatcagcattacgttcaaggaaggctcaccaaccatgaaaccgggccaatcctaccagaaccgcaatacccggagggactgctgcggcttctttcaatacagaagcaaaaacatcctctggaataactgccatatttactatatgcacggcatgggcgtcgtttcccagttctgcgagaacatcatgttcagccacttgaaaatagccccgcgcccccgttccctccgcaccaattcctcctgggcggataatctgcacttttccggatgccggggaaaaatcatcgtcaaagattgcgtgctgggagcctcccatgatgacgccgttaatgttcatggaacccacttgcgcattatagacaggccggcccccaacaaaatcaccgtccggttcatgcatccccagaccttcggctttgacgcctttgccgcaggagaccggatcgattatgtttcctgcaacacgctggtaccttatgcatccaataccgtttccggcgtcaaacaactcaatgaaaaggaaatagaacttacattgcaacatcccaatcccgggaacatccagcctgacgatgttgtggaaaacgtcacatggacgccgtccgtccacatcagcaatacggtatgccgccacattcccaccaggggcttcctgctcaccactaggaagccggtgctggttgaacggtgccggtttgaaaaaacgggcatgcccgccatcctggtggaagatgacgcctccggctggtatgaatccggcgtggtcaggaacatgaccatttcccgcaatacctttatccaatgcggagaagccgtcatccagatcgtgccgcacgctccacggccggaaggggacgttcaccggaacatcaccattaccggaaacacgtttgacctcaaaaacggaaccgccatccgcattcgccataccggtgacgtcaaggcggagaaaaacactttcaccaaagacgggaagaaaatccctgaggaaaaggcggtggatatccggtag

SEQ ID NO: 34 - protein, 596 aa, Akkermansia muciniphila
MQNPVASLLFILAMLTGPCPAADYPERTERTQSAGNHVWHIDPDKGNDGNPGTAPSTAWKSMAPANRLIMARGDTLVIHPGEHAVSLALMGEGSKQAPVTIRFMPGRHIFKHGALMTGKPQISNTNDAPNEPKAMAIRLMEAKNIRLEGKPGATDILLEGKAIFVCMEHAENVSLNGLGFDYLHPTMGEFLVTEVEGDTMKATIPDGTLYTVKDGNLTWHGPGWEFRMGGYSKVFDSASGTFQGRFDPGKTVIRELSPGKISITFKEGSPTMKPGQSYQNRNTRRDCCGFFQYRSKNILWNNCHIYYMHGMGVVSQFCENIMFSHLKIAPRPRSLRTNSSWADNLHFSGCRGKIIVKDCVLGASHDDAVNVHGTHLRIIDRPAPNKITVRFMHPQTFGFDAFAAGDRIDYVSCNTLVPYASNTVSGVKQLNEKEIELTLQHPNPGNIQPDDVVENVTWTPSVHISNTVCRHIPTRGFLLTTRKPVLVERCRFEKTGMPAILVEDDASGWYESGVVRNMTISRNTFIQCGEAVIQIVPHAPRPEGDVHRNITITGNTFDLKNGTAIRIRHTGDVKAEKNTFTKDGKKIPEEKAVDIR

SEQ ID NO: 35 - DNA, 2151 bp, Akkermansia muciniphila
  source 1..2151: mol_type = genomic DNA
  CDS 1..2151: product = AmGH36C; protein_id = 36; translation = MIPDSVHLLTRSSQMTLYKDEQGRVRRAYYGPKLREPEDALENA ADAPLLYSSLADSVTGLPNASGEYCICVTQADGALSLSLECLDWEVLELDDDREEAVF YGKDPSYPVRVEIHVRSHRESDTFLQWAVIRNEGKEGIRLHRAASAQLGLRAERYFVT SFRGTWGGESLMSEEEVARGHELALVSGTGTRTAQEGSPGFIISLDGPAREDSGEVVL GALAWSGNYRIWFRHSPYHYLFAGAGLDMAPAPYLLDGGGVFKTPPLILAHSKNGKGE ASRRIHRWARRYGLRGGESERPTLLNSWEGVYFTFTEKVLHGMMKRAADLGIELFVLD DGWFGGRFPRNDARAGLGDWQVNRAKLPHGLEDLIRQAEKLGIRFGIWVEPEMVNPHS ELYQNHPEWAIGLPHRENRLERSQYLLDLSNPHVCRYILDAMRKLLGEHPGISYVKWD CNRKISDPGSPWLDACRQGNLPIDYVRGYERILETLAAEFPDVIFQACSSGGGRADYG TMRKHHEFWTSDNTDAYERVFMQWGIGHLFPAISMAAHVTASPNHQTGRSAPLKFRFD VAMSGRLGFELQPCDMTEEDMVFSKRALAEYKRIRPVVQFGDLYRLSSPYESRVASLM YVCGKRAVVFAWLMDKWLADSPPPLRLKGLNFSARYLVREINMNEDGSLTDVHERRLG GDFLMDAGIRIRWKKAFQSVCLEVVETNPDAPAS
atgattccggattccgttcatttgctgacccgttcctctcagatgaccctgtacaaagatgaacagggccgtgtgcgccgcgcctattacggaccgaagctgcgtgaaccggaggatgccctggaaaacgccgcggatgctcctctcctgtattcctccctggcggattccgtaacagggcttcccaatgcgtccggcgagtactgcatttgcgtaacccaggcggacggggccctttccctgagcctggaatgcctggattgggaagtgctggaactggatgacgaccgggaagaagccgtcttctatgggaaggacccttcctaccctgtgcgggtggaaatccacgtgcgctcccacagggaatcggacacttttctgcaatgggcggtgattcggaacgaagggaaagaaggcatccgcctgcaccgcgcggcatccgcacagctgggtttgcgcgcagaacggtatttcgtcaccagcttccgcggcacctggggcggcgagtccctgatgagtgaggaagaagtggcccgggggcatgaacttgccctggtatccggaaccggcacccgcaccgcccaggagggaagccccggattcatcatctctctggacggtccggcgcgggaggattccggggaggtggttctgggggcgctggcatggtccggcaattaccggatatggttccggcacagcccgtatcattacctttttgccggagcggggctggatatggctcccgcgccctatctgctggacggcggcggtgtgttcaagacgccgcccctcattctggcgcacagtaaaaacggcaagggagaggcctcccggcgcatccaccgctgggcgcgccgttacggcctgcgcggtggagaatcagagaggccgaccttgctgaattcctgggagggggtgtattttacctttacggaaaaagtgctgcacggcatgatgaaacgtgcggcagacttggggatagagcttttcgtgctggatgacggctggtttggcggccggttcccccgcaatgacgcccgtgccggattgggggactggcaggttaaccgcgctaaattgcctcatggactggaagacctcattcgccaggcggaaaaactgggaatacgcttcggcatctgggtggagccggaaatggtgaaccctcattcggagctttatcagaaccatccggagtgggccatcggtctcccgcatcgggaaaacaggctggaacgcagccagtatttgctggacctgagcaatcctcatgtatgcaggtacattcttgatgccatgcggaagctgcttggggagcatccgggtatttcctacgtcaaatgggattgcaaccgcaaaatttccgaccccggctctccctggctggatgcctgccgccaggggaatctgcccattgactatgtgcggggctatgaacgtattcttgagacgctggcggcggaattcccggatgtgatattccaggcctgttcctccggaggaggccgcgccgactatggaaccatgcggaagcatcatgaattctggacgtccgacaacacggacgcttatgaacgcgtgttcatgcagtggggcataggccacctgttcccggctatttccatggccgcccatgtgaccgcctcccccaaccaccagacgggccgctctgctccgctcaaattccggtttgacgtggccatgtccggccggcttggttttgaactccagccatgtgacatgacggaggaggacatggttttttccaaacgggctctggcggaatacaagcgcatccgccccgtagtccagttcggtgacttgtaccggctttcctccccgtatgaaagccgggtggcctccctgatgtatgtatgcggaaaacgggctgtcgttttcgcctggctgatggataaatggctggcggattcccctccgcccctgcgcctgaaaggcctgaatttttccgctaggtacttggtgcgggaaatcaacatgaatgaagacggctctctcacggatgttcacgaacgaaggctgggaggcgactttctgatggacgccggcatccgcatccgctggaaaaaggccttccagtccgtctgcctggaggtggtggaaacaaaccctgatgctccggcttcatga

SEQ ID NO: 36 - protein, 716 aa, Akkermansia muciniphila
MIPDSVHLLTRSSQMTLYKDEQGRVRRAYYGPKLREPEDALENAADAPLLYSSLADSVTGLPNASGEYCICVTQADGALSLSLECLDWEVLELDDDREEAVFYGKDPSYPVRVEIHVRSHRESDTFLQWAVIRNEGKEGIRLHRAASAQLGLRAERYFVTSFRGTWGGESLMSEEEVARGHELALVSGTGTRTAQEGSPGFIISLDGPAREDSGEVVLGALAWSGNYRIWFRHSPYHYLFAGAGLDMAPAPYLLDGGGVFKTPPLILAHSKNGKGEASRRIHRWARRYGLRGGESERPTLLNSWEGVYFTFTEKVLHGMMKRAADLGIELFVLDDGWFGGRFPRNDARAGLGDWQVNRAKLPHGLEDLIRQAEKLGIRFGIWVEPEMVNPHSELYQNHPEWAIGLPHRENRLERSQYLLDLSNPHVCRYILDAMRKLLGEHPGISYVKWDCNRKISDPGSPWLDACRQGNLPIDYVRGYERILETLAAEFPDVIFQACSSGGGRADYGTMRKHHEFWTSDNTDAYERVFMQWGIGHLFPAISMAAHVTASPNHQTGRSAPLKFRFDVAMSGRLGFELQPCDMTEEDMVFSKRALAEYKRIRPVVQFGDLYRLSSPYESRVASLMYVCGKRAVVFAWLMDKWLADSPPPLRLKGLNFSARYLVREINMNEDGSLTDVHERRLGGDFLMDAGIRIRWKKAFQSVCLEVVETNPDAPAS

SEQ ID NO: 37 - DNA, 1422 bp, Akkermansia muciniphila
  source 1..1422: mol_type = genomic DNA
  CDS 1..1422: product = AmGH109A; protein_id = 38; translation = MSIFSSRRQFLKSLGLAAGAAAAGNALPGKAVEIPAGDHLWKSA SPAAPRPSGSTYMGGFKAPRLGRIRLAFIGVGGRGFSHLAQMCVMDGVEIVGICDLKE ELTKRGVDRVLSRMGKSPLGYSGGDMEYLTMLKELKPDAVIISTDWSSHARIACDSMK HGAHAFVEVPLAVSLEELWSLVDTSEATRKHCMMMENVNYGRDELMFLNMVRQGVIGD LLHGEAAYIHCLVTQLGDTRGEGAWRPEYHTRINGNLYPTHGLGPVAQYMNLERGEDR FCRVAAFASPALGRNAYAKKHLPADHRWNNTPFICGDMNTAVVKTQLGRTILVQLDET SPRPYSRANLIQGTEGTLAGFPTRVAGEKLGNGNYHEWIEGREKLAAIYEKYDHPLWK RIGELATKMGGHGGMDFVMLSRIVECLRNGEPMDQNVYEGASWSSLLPLTARSIAQGG MPVEFPDFTRGDWKTTMPLAVVS
atgtctattttctcatcacgcagacaatttctcaaatctttggggcttgcggccggagcggctgccgccggaaatgccctccctgggaaggctgtggaaatccctgccggagaccatctctggaaatccgcctctccggcggctccgaggccttccggttccacatacatgggagggttcaaggctccccggctgggtcgcatcaggctggccttcatcggcgtgggagggcgcgggttctcccacctggcgcaaatgtgcgtgatggatggagtggaaatcgtgggcatatgtgatttgaaggaagagttgacgaaacgcggcgtggatcgcgtgctctccagaatggggaaaagccctttgggctattccggcggcgatatggaatacctgaccatgctgaaggagctgaagccggatgccgtcatcatcagtacggattggagttcgcatgccagaatcgcctgcgacagcatgaagcacggcgctcacgcctttgtggaagttcctctggccgtctctctggaggagctctggagcctggtggataccagcgaggccaccaggaaacattgcatgatgatggaaaacgtcaactatgggcgggatgaactcatgttcctgaacatggtccggcagggcgtcatcggcgatttgcttcacggggaggccgcgtatatccattgcctggtgacgcagctgggggacacgcgcggggaaggggcctggcggccggaatatcataccagaatcaatggcaacctgtaccccacccacgggttggggccggtggctcaatatatgaatttggagcgtggagaggaccgtttctgccgtgtggcggcgttcgcttctcctgctctcgggcgcaatgcctacgctaaaaagcatcttcccgccgatcaccgctggaacaatactccattcatctgcggtgacatgaatacggctgttgtcaagacgcagctggggcggaccattcttgtccagctggatgagacgtccccccggccttactcccgcgccaacctgatccagggaacggagggcacgctggctggtttcccaacccgcgtggcgggtgaaaagctgggcaacggcaattatcatgaatggattgaaggcagggaaaaactggccgctatttatgaaaaatacgatcatcccctctggaaacgcatcggggagctggccacgaaaatgggcggtcacggcggtatggactttgtgatgctttcccgcatcgtggaatgcctccggaacggagaaccaatggatcagaacgtttacgaaggagcttcctggtcttccctgctgccgttgacagcccgttccatcgcccagggcgggatgcctgtggaatttccggattttacccgcggagactggaaaaccaccatgccgctggccgtggtttcatga

SEQ ID NO: 38 - protein, 473 aa, Akkermansia muciniphila
MSIFSSRRQFLKSLGLAAGAAAAGNALPGKAVEIPAGDHLWKSASPAAPRPSGSTYMGGFKAPRLGRIRLAFIGVGGRGFSHLAQMCVMDGVEIVGICDLKEELTKRGVDRVLSRMGKSPLGYSGGDMEYLTMLKELKPDAVIISTDWSSHARIACDSMKHGAHAFVEVPLAVSLEELWSLVDTSEATRKHCMMMENVNYGRDELMFLNMVRQGVIGDLLHGEAAYIHCLVTQLGDTRGEGAWRPEYHTRINGNLYPTHGLGPVAQYMNLERGEDRFCRVAAFASPALGRNAYAKKHLPADHRWNNTPFICGDMNTAVVKTQLGRTILVQLDETSPRPYSRANLIQGTEGTLAGFPTRVAGEKLGNGNYHEWIEGREKLAAIYEKYDHPLWKRIGELATKMGGHGGMDFVMLSRIVECLRNGEPMDQNVYEGASWSSLLPLTARSIAQGGMPVEFPDFTRGDWKTTMPLAVVS

SEQ ID NO: 39 - DNA, 2367 bp, Akkermansia muciniphila
  source 1..2367: mol_type = genomic DNA
  CDS 1..2367: product = AmGH95A; protein_id = 40; translation = MNVPATLKHSFLLCLFAAAVPLRAIPAPMQVTASTPARVWTEGY GTGNGRLGILSFGVFPKETVVLNEGSIFAKKNFQMREGAAEALDKARELCKEGKYRSA DQLFRKNILPPGNIAGDYQQGGRLQVEFQGLPSPSSYQRTLDMRRGKATTRAQFGTGE LTTEILAAPSSDCAAYHIACTMPSGCRVSLNLEHPDPSARIVAQPNGWVLEGQGSNGG TRFENTVVILAPGASVTRKGSTIILDSAREVMVLSSISTDYNIRKPEAPLTHSLAAKN ARILAKAQKAGWKKLAAETEDYFSRLMTRCQVDLGDSPAGVSAMTTAQRLERVKQGKK DPDLLEQLFQFGRFCTIAHTRPGQLPCGLQGLWNPELRAAWMGCYFLNINSQMNQWPS HVTGLGEFQSSYLDFVRSLRPHGEEFARFIKRDGFCFGHYTDCWKRTYFSGNNPEWGA SLMNGAWACAHLVDSYRFTGDREDLKKSLPILESNARFIMSWFEDDGEGRYLSGPGVS PETGFYAPDGTGPNVLSYVSNGTSHDQLLGREALRNYIYACGELGIRTPTLLKAVQFL RKIPQPAIGPDGRVQEWRQPFEEMQKGHRHISHLYGLFPGTEWDVLNTPEYAEAVRKS ADFRRKYADMGNNGIRTGWSTAWLINLYAALGDGNAAEDRMYTMLRHYINSNLFDLHP PFQIEGNFGFSSGVAECLIQSRIMQDGFQVILLAPALADDWKKGSATGLRTRGGLKVD LSWQDGRVQATATAARPGKFRFMHQGRKKDLSMNKGETVRLDFPPLSH
atgaacgtccctgcaaccttgaaacattccttcctgctctgccttttcgcggcagccgtccccctccgggccattccggcccccatgcaggtcaccgcctccacccccgcccgggtatggacggaaggatacggcacgggcaacggaaggctgggcatcctctctttcggcgtttttccgaaggaaaccgtcgtcctcaatgaaggaagcattttcgcaaaaaaaaatttccaaatgagggaaggcgccgcggaagctctggacaaggcgcgcgaactttgcaaggaaggaaaataccgcagtgcagaccagctattccgtaagaatatccttccccccggcaacattgcgggggattaccagcagggggggcgtcttcaggtggaattccaggggcttccttcaccttcctcctatcagcgtacactggatatgcgacggggcaaggcaaccacccgcgcccaattcggcacgggggaattgaccacagaaatcctggcggccccgtccagcgactgcgccgcctaccacattgcctgcacgatgccgtccggatgccgcgtttccctgaatctggaacacccggatccgtccgcccgaattgttgcgcagccgaatggctgggtgctggaaggacagggaagcaacggaggcacccggtttgaaaacacggtggtcatcctggcgcccggagcctccgttacccgcaaaggctcaacaattattcttgattccgcccgggaagtgatggtgctttcatccatttccacagattacaatatccggaagccggaagctcccctgacgcattccctggcggctaaaaacgcgagaatcctggcaaaagcgcaaaaggcgggatggaaaaaactggcggcggaaacggaagattatttttcccggctcatgacgcgctgtcaggtggatctgggggattccccggccggggtttccgccatgaccaccgcccaaaggctggagcgggtcaaacagggaaagaaggatccggatcttctggaacagcttttccagttcggccgcttctgcacgattgcccacaccaggccggggcagcttccctgcggtttgcagggattatggaatccggagctgagggcggcgtggatgggctgctatttcctgaatatcaacagccagatgaaccagtggccctcccatgtcaccgggctgggagaattccagagctcctatcttgattttgtccgcagcctgcgcccccacggagaggagtttgcgcgcttcatcaaacgggacggcttctgcttcggccactatacggattgctggaagcgcacctatttttcaggcaacaatccggaatggggggccagcctcatgaacggggcatgggcatgcgcccacctggtggacagttaccgctttaccggagaccgggaagaccttaaaaaatcgctgcccattctggaatccaacgcgcgtttcatcatgtcctggtttgaagacgacggcgagggccgttacctctccggcccgggggtgtctccggagaccggattttacgcgccggacggcacaggccccaacgtgctttcctatgtttccaacggcacgtcccatgaccagcttctgggcagggaggctctccgcaattatatctacgcctgcggagagctgggcataaggactcccaccctgcttaaagccgtccaatttctcaggaaaatcccccagcctgccatcggtcccgacggaagggtgcaggaatggaggcagccttttgaagaaatgcagaagggccaccggcacatcagccacctttacggcctgttccccggcacggaatgggatgttctcaatactccggaatatgcggaggccgtacgcaagtcagccgatttccggcgcaagtacgcggatatgggaaacaacggcatcaggacgggatggagcacggcgtggctcatcaatctttatgccgcccttggcgacgggaacgccgcagaggacaggatgtacaccatgctgaggcactacatcaactccaacctttttgacctccatcccccgttccagatagaagggaatttcggcttttcctccggcgtggccgaatgcctgatccagagccggatcatgcaggacgggttccaggttatcctgctggctccggccctggcagacgactggaagaaggggtctgccacgggattgcgcacgcggggagggcttaaggtggacttgtcctggcaagacggccgggtgcaggccacggctaccgctgcgcgtccgggcaaattccggttcatgcatcagggccggaaaaaggatttgtccatgaataagggagaaacggtgcgtcttgattttcctccgctttcccattag

SEQ ID NO: 40 - protein, 788 aa, Akkermansia muciniphila
MNVPATLKHSFLLCLFAAAVPLRAIPAPMQVTASTPARVWTEGYGTGNGRLGILSFGVFPKETVVLNEGSIFAKKNFQMREGAAEALDKARELCKEGKYRSADQLFRKNILPPGNIAGDYQQGGRLQVEFQGLPSPSSYQRTLDMRRGKATTRAQFGTGELTTEILAAPSSDCAAYHIACTMPSGCRVSLNLEHPDPSARIVAQPNGWVLEGQGSNGGTRFENTVVILAPGASVTRKGSTIILDSAREVMVLSSISTDYNIRKPEAPLTHSLAAKNARILAKAQKAGWKKLAAETEDYFSRLMTRCQVDLGDSPAGVSAMTTAQRLERVKQGKKDPDLLEQLFQFGRFCTIAHTRPGQLPCGLQGLWNPELRAAWMGCYFLNINSQMNQWPSHVTGLGEFQSSYLDFVRSLRPHGEEFARFIKRDGFCFGHYTDCWKRTYFSGNNPEWGASLMNGAWACAHLVDSYRFTGDREDLKKSLPILESNARFIMSWFEDDGEGRYLSGPGVSPETGFYAPDGTGPNVLSYVSNGTSHDQLLGREALRNYIYACGELGIRTPTLLKAVQFLRKIPQPAIGPDGRVQEWRQPFEEMQKGHRHISHLYGLFPGTEWDVLNTPEYAEAVRKSADFRRKYADMGNNGIRTGWSTAWLINLYAALGDGNAAEDRMYTMLRHYINSNLFDLHPPFQIEGNFGFSSGVAECLIQSRIMQDGFQVILLAPALADDWKKGSATGLRTRGGLKVDLSWQDGRVQATATAARPGKFRFMHQGRKKDLSMNKGETVRLDFPPLSH

SEQ ID NO: 41 - DNA, 1113 bp, Roseburia intestinalis
  source 1..1113: mol_type = genomic DNA
  CDS 1..1113: product = RiFuc95; protein_id = 45; translation = MDGLLIPGVSTIDGKPMGGWSQYALSPTMSIWVALSFDEYYLYT GDVLFLKEKAYPFFYEVGKAIMGLLEEKNGKLYLPLSTSPEIYDDTPEAYLIPNSNFD LALIRYLFVRLQEYSEILNKEKTAYEEILSKLDEIAVIDGYIGLNRVERLKESHRHFS HLMCLYPLHLIHYDTKEHRELYKKALSEIEKLGTGMWVGFSFAMCAQLYAMAENGAGA YEKLRQFADGFVEENGFHLNGDYKQKGYSTFHYRRFTLEALFGFCDALQEMLLQEYRG YLHLFPAIPKQWENEGIEFNGFRSYGGILVSGKIKNGYLSEIRLKIPEKMLIKIKNTF PTKRARLETDKKTQIIEETEGFFVLELEKGNAKITG
atggatggtctgctcattccgggtgtgtcaacgattgacggaaaaccaatgggtggctggtcacaatatgcactttctccaactatgagtatctgggtggcactaagctttgatgagtattatttgtatacaggtgatgttctttttttaaaagagaaggcatatccatttttttatgaagttggaaaggccattatgggtcttttagaggaaaagaatgggaaattatatttgccactctccacctcaccggaaatatatgatgatacgccagaggcatatttgatcccaaacagtaattttgatcttgcacttatcagatacttgtttgtgcggttgcaggagtattcagaaatattaaataaggaaaaaacagcatatgaagagatactttctaaattagatgagattgcagttatagatggatatatcggcttgaacagagtagaacgtttgaaagagtcacacaggcatttttcacatttaatgtgtctgtatccgcttcatttgatacattatgatacaaaagaacacagggaactttataaaaaagccttatcagaaatagagaaactcggaactggaatgtgggtaggattttcgttcgcaatgtgtgcacagctctatgctatggcagagaatggagcaggcgcttatgaaaaactgagacaatttgcggatggatttgtagaagaaaacggatttcacctgaatggtgattacaagcaaaaaggttattcaacctttcattacagacgttttacgttggaagcattatttggattttgtgatgcattacaagagatgcttttacaggaatatcggggatatttgcatttatttccagcaattccaaaacaatgggaaaacgaggggatagaatttaatggttttcggagttatggtggtattctcgtaagcggaaaaattaaaaatggttatctgtcagaaatccgattgaagatacctgagaaaatgcttataaaaataaaaaatacatttccaacaaaaagagccagactggagacagacaagaaaacgcagataatagaggaaacagaaggattttttgttttagagttagaaaaaggaaatgcaaaaattacaggataa

SEQ ID NO: 42 - protein, 370 aa, Roseburia intestinalis
VDGLLIPGVSTIDGKPMGGWSQYALSPTMSIWVALSFDEYYLYTGDVLFLKEKAYPFFYEVGKAIMGLLEEKNGKLYLPLSTSPEIYDDTPEAYLIPNSNFDLALIRYLFVRLQEYSEILNKEKTAYEEILSKLDEIAVIDGYIGLNRVERLKESHRHFSHLMCLYPLHLIHYDTKEHRELYKKALSEIEKLGTGMWVGFSFAMCAQLYAMAENGAGAYEKLRQFADGFVEENGFHLNGDYKQKGYSTFHYRRFTLEALFGFCDALQEMLLQEYRGYLHLFPAIPKQWENEGIEFNGFRSYGGILVSGKIKNGYLSEIRLKIPEKMLIKIKNTFPTKRARLETDKKTQIIEETEGFFVLELEKGNAKITG

SEQ ID NO: 43 - DNA, 2343 bp, Akkermansia muciniphila
  source 1..2343: mol_type = genomic DNA
  CDS 1..2343: product = AmGH35B; protein_id = 44; translation = MKLSFFSVLLLAGHLCAAAPMPLPESNDGARHVFSTNQENFLMD GKPVKIISGEMHYPRVPRQHWKDRFQRIKAMGMNTVCTYLFWNVHEPEPGKWDFSGNL DFVEFIKEAQKAGLWVIVRPGPYVCAEWEFGGFPGWLLKDEDLKVRSQDPRFLEPAMA YLKKVCSMLEPLQITKGGPIIMAQVENEYGSYGSDKDYVKKHLDVIRKELPGVVPFTS DGPNDWMIKNGTLPGVVPAMNFGGGAKGAFANLEKHKGKTPRINGEFWVGWFDHWGKP KNGGSTEGFNRDLKWMLENNVSPNLFMAHGGTSFGFMNGANWEGAYTPDVTNYDYGAP ISENGTLTDRYRTFRQTIQDYYGDTYKLPEPPAQPEMMELPPITFTETAGMFSRLPQP VIRKEPVHMEALGQSLGFILYRTKVNGPVKGELKMNNMQDRAIVYVDGKRQGAADRRY KQDSCDIVIPSGLHTVDIFVENMGRINFGGQIQGERKGIRGPITLDGKKLENFLIYNF PCKGVELIPFSGKKPAGDQPVFHRGYFNVSNPKDTYLDMRDGWKKGVVWVNGRNLGRF WFIGSQQALYCPGEYLKPGKNEIVVLDVDGGSGTVKGVKEAIYEVNRDPAMADVFRVG KPVAPAAGQLVHKGSFAKGADQQEIKFRAPVQARYIAIVSKNAHDNGPHAAIAELNFL DASGNLLPREQWSVVYADSHETTGEAAQAGLVMDNQPTTYWHTKWQGDNPRHPHMIVL DLGKVQKLSGFRYLPRQDRENGRIKDYEVYASPKPFKPAK
atgaaattatcctttttctccgttctgcttctggcagggcatctttgtgcggctgctcccatgcctttgccggaatccaatgacggagccaggcatgtcttctctactaatcaggaaaattttttgatggacggaaagcccgtcaaaatcatttccggggaaatgcattatcctcgcgtgccgcgccagcactggaaggacaggttccagcgcataaaggccatgggcatgaataccgtctgcacttatctgttctggaacgtgcatgaaccggaacccggcaaatgggacttttccggcaatctggattttgtggaattcatcaaggaggcgcagaaggccggcctgtgggtcattgtgcgtcccgggccctatgtgtgcgcggaatgggaattcggcggatttcccggctggctgctgaaggatgaagatttgaaagtccgttcccaggatccccgcttcctggaaccggccatggcttatcttaaaaaagtctgttccatgctggaacctctgcagattaccaagggaggccccatcatcatggcccaggtggaaaatgaatacggctcctatggttctgacaaggattacgtgaaaaagcatctggacgttatccggaaagaacttccgggagttgttcccttcacgtcggacggcccgaacgactggatgatcaagaacggcacgcttccgggcgttgttcccgccatgaatttcggcggcggagccaagggcgcttttgcgaatctggagaagcacaagggcaaaacgccccgcatcaacggcgaattctgggtgggctggtttgaccactggggcaagcccaagaatggcggcagtacggaaggtttcaaccgagacctgaagtggatgctggaaaataacgtttcccccaacctattcatggcgcatggggggacctccttcggcttcatgaacggggcgaactgggaaggcgcctacacgccggatgtaaccaattacgactacggcgcccccatttccgaaaacggaaccctgacggaccgctaccgcaccttccgccagactattcaggattattacggtgatacgtacaagcttcccgaacctcccgcccagccggaaatgatggagctgcctcccatcacgtttacggaaacagccggcatgttctcccgcctgccgcagcccgtcatccgcaaggagcccgtccacatggaagccttggggcaaagcctgggcttcatcctgtaccggacaaaggtgaacggcccggtgaaaggagagctgaagatgaacaacatgcaggaccgcgccatcgtttacgtggacggcaagaggcagggggcggcggaccgccgttacaagcaggattcctgtgacattgtcattccctccggacttcacacggtggacatttttgtggaaaacatgggccgcatcaacttcggcggccagatacagggcgagcgcaagggcatccggggccccattacgctggacggcaaaaagctggaaaacttccttatctacaacttcccgtgcaagggggtggagcttattcccttctccggcaagaagccggcgggcgaccagcccgtgttccaccgcgggtatttcaacgtttccaatcccaaggatacctacttggatatgcgggacggctggaagaaaggcgtcgtgtgggtgaatggacgcaatctgggccgcttctggtttatcggctcccagcaggctctttattgccccggagaatacctgaagcccgggaaaaatgaaatcgtggtgctggacgtggacggaggttccggcacggtgaagggtgtgaaggaagccatttatgaagtcaacagggaccccgccatggcggatgtcttccgcgtgggcaaacctgtggcccccgctgccggccagctggtgcacaagggttccttcgccaagggggcggaccagcaggaaatcaaattccgcgctcctgtccaggcccgttacatagctattgtcagtaaaaacgctcatgacaacggcccccatgccgccattgcggagctgaacttcctggatgcctccggcaatctgctcccccgcgaacagtggtccgtggtttatgcggattcccatgaaacgacgggagaagccgcccaggcgggactggtgatggacaaccagcccaccacctactggcataccaagtggcagggggacaaccccaggcatccgcacatgatcgtgctggatctgggcaaggtgcagaaactttcaggattccgctacctgccgcgccaggaccgggaaaacggccgcatcaaggactatgaagtctatgcgtctcccaagccgttcaagcctgccaagtaa

SEQ ID NO: 44 - protein, 780 aa, Akkermansia muciniphila
MKLSFFSVLLLAGHLCAAAPMPLPESNDGARHVFSTNQENFLMDGKPVKIISGEMHYPRVPRQHWKDRFQRIKAMGMNTVCTYLFWNVHEPEPGKWDFSGNLDFVEFIKEAQKAGLWVIVRPGPYVCAEWEFGGFPGWLLKDEDLKVRSQDPRFLEPAMAYLKKVCSMLEPLQITKGGPIIMAQVENEYGSYGSDKDYVKKHLDVIRKELPGVVPFTSDGPNDWMIKNGTLPGVVPAMNFGGGAKGAFANLEKHKGKTPRINGEFWVGWFDHWGKPKNGGSTEGFNRDLKWMLENNVSPNLFMAHGGTSFGFMNGANWEGAYTPDVTNYDYGAPISENGTLTDRYRTFRQTIQDYYGDTYKLPEPPAQPEMMELPPITFTETAGMFSRLPQPVIRKEPVHMEALGQSLGFILYRTKVNGPVKGELKMNNMQDRAIVYVDGKRQGAADRRYKQDSCDIVIPSGLHTVDIFVENMGRINFGGQIQGERKGIRGPITLDGKKLENFLIYNFPCKGVELIPFSGKKPAGDQPVFHRGYFNVSNPKDTYLDMRDGWKKGVVWVNGRNLGRFWFIGSQQALYCPGEYLKPGKNEIVVLDVDGGSGTVKGVKEAIYEVNRDPAMADVFRVGKPVAPAAGQLVHKGSFAKGADQQEIKFRAPVQARYIAIVSKNAHDNGPHAAIAELNFLDASGNLLPREQWSVVYADSHETTGEAAQAGLVMDNQPTTYWHTKWQGDNPRHPHMIVLDLGKVQKLSGFRYLPRQDRENGRIKDYEVYASPKPFKPAK

SEQ ID NO: 45 - protein, 370 aa, Roseburia intestinalis
MDGLLIPGVSTIDGKPMGGWSQYALSPTMSIWVALSFDEYYLYTGDVLFLKEKAYPFFYEVGKAIMGLLEEKNGKLYLPLSTSPEIYDDTPEAYLIPNSNFDLALIRYLFVRLQEYSEILNKEKTAYEEILSKLDEIAVIDGYIGLNRVERLKESHRHFSHLMCLYPLHLIHYDTKEHRELYKKALSEIEKLGTGMWVGFSFAMCAQLYAMAENGAGAYEKLRQFADGFVEENGFHLNGDYKQKGYSTFHYRRFTLEALFGFCDALQEMLLQEYRGYLHLFPAIPKQWENEGIEFNGFRSYGGILVSGKIKNGYLSEIRLKIPEKMLIKIKNTFPTKRARLETDKKTQIIEETEGFFVLELEKGNAKITG

(19) United States
(12) Patent Application Publication
(10) Pub. No.: US 2026/0317714 A1
(43) Pub. Date: Oct. 15, 2026

(54) ENZYME-CONVERTED RED BLOOD CELLS FOR INCREASED COMPATIBILITY OF ABO-UNIVERSAL BLOOD TRANSFUSION

(71) Applicants: DANMARKS TEKNISKE UNIVERSITTET, Kongens Lyngby (DK); LU License AB, Lund (SE)

(72) Inventors: Maher Abou HACHEM, Espergaerde (DK); Mathias JENSEN, Kongens Lyngby (DK); Linn STENFELT, Kongens Lyngby (DK); Martin Lennarth OLSSON, Lund (SE); Jennifer Ricci HAGMAN, Kongens Lyngby (DK); Annika K. HULT, Kongens Lyngby (DK)

(21) Appl. No.: 19/484,914

(22) PCT Filed: May 16, 2024
(86) PCT No.: PCT/EP2024/063626

§ 371 Date: Nov. 17, 2025

(30) Foreign Application Priority Data
May 17, 2023 (EP) 23174126.5
Jan. 4, 2024 (EP) 24150419.0

(51) Int. Cl. C12N 5/078; A61K 35/18; C12N 9/24

(52) U.S. Cl. CPC C12N 5/0641; A61K 35/18; C12N 9/2402; C12Y 302/01023; C12Y 302/01049; C12Y 302/01053; C12Y 302/01063; C12N 2501/73

(57) ABSTRACT

The invention relates to securing a sufficient supply of blood having substantially reduced levels of A antigens, and the extended Gal-A, H type 3, A type 3 antigens as well as B antigens and the extended GalNAc-B antigens that can be used to supplement supplies of donor-derived blood group O red blood cells. The invention provides enzyme-converted red blood cells, and enzyme cocktails for converting extended A-type and/or −B-type antigens, in addition to A-, and B-antigens on the surface of donor red blood cells.

### FIELD OF THE INVENTION

The invention relates to securing a sufficient supply of blood having substantially reduced levels of A antigens, and the extended Gal-A, H type 3, A type 3 antigens as well as B antigens and the extended GalNAc-B antigens that can be used to supplement supplies of regular, donor-derived blood. The invention provides enzyme-converted red blood cells (RBCs), and enzyme cocktails for converting the extended A-type and/or B-type antigens, in addition to A- and B-antigens on the surface of A or B (or AB) RBCs. The enzyme-converted RBCs lack the A antigens, and the extended Gal-A, H type 3, A type 3 antigens as well as B antigens and the extended GalNAc-B antigens, which underpin the incompatibility of unconverted RBC for transfusion into ABO-incompatible recipients.

### BACKGROUND OF THE INVENTION

There is a blood supply challenge due to a decline in donor numbers and excessive use of universal blood type O. The ABO blood system is the prime factor to assess transfusion incompatibility. Matching blood types of donor cells and with recipient plasma is crucial to avoid acute and sometimes deadly hemolytic transfusion reactions. This incompatibility stems from positive crossmatches between naturally-occurring recipient antibodies recognizing non-self ABO antigens on donor RBCs. Maintaining stocks of only the ABO-universal type O blood, offers an attractive remedy to this challenge and would also solve logistic challenges for centralized transfusion services when serving satellite blood centers and hospitals. The enzymatic conversion of type A and B blood to the universal type O (ECO-blood), pioneered in 1982, made use of a coffee-bean derived α-galactosidase to convert the B antigen to its precursor H antigen. However, this enzyme was inefficient for feasible conversion and lacked ability to convert A antigen to H antigen. Despite the inefficiency of this enzyme, the clinical feasibility of this system was, however, demonstrated through apparently successful ECO-blood transfusion in early clinical trials, but this was overshadowed by weak unexplained crossmatch reactivities, which raised safety concerns regarding transfusion compatibility.

Later, more efficient enzymes to convert both A and B to H antigen were identified. Despite a more efficient conversion process of RBCs, unexplained and sometimes strong incompatibilities remained, particularly against plasmas from type O individuals. The present invention addresses the need to provide a source of ABO-universal blood enzymatically converted to type O (ECO-blood) and the enzymes needed to secure the conversion of type A and B blood to eliminate the epitopes associated with type A, B and AB, and uniquely for the first time all their known extensions that may cause cross-match reactivities, to create ABO-universal blood similar to type O or the Bombay type lacking H, both expected to be universally compatible.

### SUMMARY OF THE INVENTION

In a first embodiment, the invention provides a population of enzyme-converted ABO-compatible RBCs having no detectable A-antigens, B-antigens, extended A- and extended B-antigens as determined by serological typing, wherein said enzyme-converted ABO-compatible RBCs are obtained by enzymatic removal of A-, B-antigens, extended A- and extended B-antigens on RBCs.

In a further aspect, the invention provides a blood product comprising the population of enzyme-converted ABO-compatible RBCs according to the invention, wherein said cells are reconstituted as a cell suspension in a medium suitable for transfusion.

In a second embodiment, the invention provides an enzyme composition for enzyme-conversion of A or B (or AB) RBCs to enzyme-converted RBCs according to the first embodiment, comprising purified enzymes characterized as:
(i) a β-1,3-N-acetylgalactosaminidase belonging to phylogenetic cluster II or VII of GH20 family, and
(ii) an α-1,3-galactosidase belonging to phylogenetic cluster II of GH110 family and/or purified enzymes characterized as:
(iii) an α-1,3-N-acetylgalactosaminidase belonging to phylogenetic cluster I of GH36 family or phylogenetic cluster II of GH109 family,
(iv) an α-1,2-fucosidase belonging to phylogenetic cluster III of GH95 family and (v) a β-1,3-galactosidase belonging to phylogenetic cluster IV of GH35 family.

In a third embodiment, the invention provides a method for removing A-antigens, B-antigens, extended A- and extended B-antigens from A or B (or AB) RBCs comprising the steps of:
(i) contacting A or B (or AB) RBCs with an enzyme composition according to the second embodiment under isotonic, low ionic strength and neutral pH conditions, as maintained by a conversion buffer, for a period sufficient to remove the A-antigens, B-antigens and extended A- and extended B-antigens, and
(ii) depleting the product of step (i) for purified enzymes of said enzyme composition,
wherein said enzyme composition is capable of removing all detectable A-antigens, B-antigens and extended A- and extended B-antigens from said RBCs, or at least reducing the level of said detectable A-antigens, B-antigens and extended A- and extended B-antigens on said RBCs relative to corresponding untreated RBCs and/or relative to RBCs treated with enzymes that remove only A-antigens and/or B-antigens.

In a fourth embodiment, the invention provides for the use of the composition according to the second embodiment for enzymatic removal of A-antigens, B-antigens and extended A- and extended B-antigens from A or B (or AB) RBCs.

In a fifth embodiment, the invention provides a population of enzyme-converted ABO-compatible RBCs, or a blood product derived therefrom according to the first embodiment for use as a medicament.

In a sixth embodiment, the invention provides a population of enzyme-converted ABO-compatible RBCs, or a blood product derived therefrom according to the first embodiment for use in treatment of a human suffering from suffering from anemia requiring transfusion, e.g. due to loss of RBCs, increased destruction of RBCs and/or decreased production of RBCs.

### DESCRIPTION OF THE INVENTION

### Brief Description of the Figures

FIG. 1. A cartoon showing the structure of the ABO blood group antigens: a) The B antigen, an H type 2 antigen (O type), and an A antigen. The shown A and B antigens are on the RBC-dominant H type 2 antigen, and thus, are also referred to as B type 2 and A type 2 antigens, respectively. Similar A, B and H antigens occur in small amounts on RBCs as other types, mainly type 1. The Bombay type antigen, which occurs naturally, e.g. from lower or impaired activity of the fucosyltransferase that adds the terminal α-1,2-fucosyl of the H antigen or can be generated by the enzymatic defucosylation. b) A GalNAc-B extended B-antigen comprising a β-(1,3)-N-acetylgalactosaminyl extension of the nonreducing-end galactosyl unit of the B antigen. c) Three extended A-antigens characterised by the consecutive non-reducing end extension with a β-1,3-galactosyl (Gal-A), an α-(1,2)-fucosyl (H type 3) assembled on the galactosyl of the H-type 3 antigen, and an α-1,3-N-acetylgalactosaminyl unit assembled on the H-type 3 antigen galactosyl unit. d) Additional H type antigen backbones, present at lower abundances as compared to the H type 2 type on RBC surfaces. The cartoon further shows the enzymatic specificities needed for each conversion and respective enzymatic reactions (indicated by arrows), which result in the conversion of the ABO blood group antigens to the universal non-antigenic O type or the Bombay type precursor.

FIG. 2A. The enzymatic removal of B-antigens and extended GalNAc-B antigens from blood group B RBCs by the A. muciniphila α-1,3-galactosidase (AmGH110A) and β-N-acetylgalactosaminidase (AmGH20A), respectively. Solid grey histograms show levels of the analysed B antigen and extended B [GalNAc-B]antigen, detected on RBCs by fluorescent immunostaining, using an anti-B antigen antibody (Ab, clone 9621A8) and secondary rat-anti-mouse kappa (RAMκ)-phycoerythrin (PE) antibody. The advantage of the fluorescence signal from PE is that it is resolved from cell-fluorescence, which results in higher signal to noise ratio. Extended B [GalNAc-B]antigen was detected using a human eluate of anti-GalNAc-B and the goat-anti-human (GAH)-IgG/GAH-IgM-PE mix as primary and secondary Abs, respectively, in flow cytometry analysis. The dotted black line histograms show O type RBCs as negative controls for evaluation of the enzymatic conversions. a) The glycan structure of the antigens detected in panels b) and c) following treatment with AmGH110A. d) The glycan structure of the antigens on untreated native B RBCs as controls. e) and f) represent the histograms corresponding to the structures shown in panel d). g), h) and i) The glycan structure of the antigens on B RBCs after treatment with AmGH20A and the corresponding histograms. j), k), and I) the glycan structure of the antigens on B RBCs after sequential treatment with AmGH20A and AmGH110A and the corresponding histograms. m) The glycan structure of the antigens on B RBCs after the simultaneous one-pot treatment with both AmGH110A and AmGH20A and the corresponding histograms (n-o). The figure shows that treatment with AmGH110A is not sufficient for the full conversion of blood group B RBCs, but rather requires treatment with both AmGH110A and AmGH20A (βanels j-o).

FIG. 2B. Enzymatic removal of B- and extended GalNAc-B antigens from blood group B RBCs using A. muciniphila α-1,3-galactosidase (AmGH110A) and β-N-acetylgalactosaminidase (AmGH20C), respectively. The left panel indicates the schematic removal of the non-reducing saccharide units, according to the immunostained RBCs (B staining: 9621A8)/anti-RAMκ-phycoerythrin (PE) and extended GalNAc-B staining: Human anti-GalNAc B eluate/GAH-IgG/GAH-IgM) in the grey histograms on the right as analysed by flow cytometry. The dotted histograms are negative control (O RBCs) for comparison. Type B RBCs in native state (a-c), post treatment with AmGH110A (d-f) or a combination of AmGH20C and AmGH110A (g-i).

FIG. 3. Enzymatic conversion of the A-type antigens and their extended forms on A₁ phenotype RBCs. In the left panel, a schematic presentation of the sequential and simultaneous one-pot enzymatic transformation of the antigens of interest, according to immunostained RBCs (using ES-15/RAMκ-Phycoerythrin(PE) for staining of all A antigen types, TH1/RAMκ-PE for specific staining of A type 3 antigen, BRIC231/RAMκ-PE for staining all types of H antigen, HH14/RAMκ-PE for specific staining of H type 3 antigen and 3C9/RAMκ-PE for staining of Gal-A) analyzed by flow cytometry as shown in the histograms on the right. Native RBCs stain positive for the A antigen, where only the most common A type 2 chain (see FIG. 1a) is shown for simplicity, besides the extended A type 3 antigen. a) Representative histograms of native A₁ phenotype RBCs without enzymatic treatment (b-f) or after sequential treatment with AmGH36A (h-l), AmGH95B (n-r), AmGH35A (t-u), and AmGH36A (w), or a one-pot treatment with all three enzymes (y-ac). The blood group A₁ phenotype has the highest expression of A antigens on RBCs and is thus considered the index against which all the other variants are compared. The solid grey histograms from the flow cytometry analysis depict the untreated (a-f) and enzyme-treated (h-ac) A₁ RBCs, while the dotted line is the negative control of each staining (group O RBCs for anti-A, anti-A type 3, anti-H type 3 and anti-Gal-A, and Bombay RBCs for anti-H).

FIG. 4 a,b Crossmatch test of AmGH36A-treated A₁ RBCs as compared to simultaneous one-pot treatment using AmGH36A, AmGH95B and AmGH35A with RBCs from two different donors, one secretor (expected to carry A type 1-4) and one non-secretor (expected to carry A type 2-4). Reactivity was graded as positive, 1+ to 4+[where the weakest is denoted (1+) and the strongest has the highest number], negative, −[where suggestion of a possible reaction is denoted (−)] and mixed field, m, i.e. if a double population was seen and interpretation of result could not be done. a) The reactivity of O type plasmas (n=100) with donor RBCs post treatment with AmGH36A or the enzyme cocktail. AmGH36 treatment alone eliminated positive crossmatches for 17% of the tested plasmas (which otherwise displays severe crossmatch reactions between 3+ to 4+), whereas treatment with the enzyme cocktail that targets the extended A structures abolished positive crossmatches in 48% of the plasmas (otherwise severely crossmatch reactive without enzyme treatment). By comparison, AmGH36 treatment alone eliminated positive crossmatches in 20% of treated non-secretor plasmas, whereas treatment with the enzyme cocktail abolished positive crossmatches in 53% of the same plasmas. b) Comparison of the mean reactivity with the enzyme treatments shown in a). Without treatment, all (100%) of the plasmas exhibit strongly positive crossmatches, whereas treatment with AmGH36A resulted in significant decrease of crossmatch reactivity, which was further decreased by the one-pot treatment with the three-enzyme blend. Reactions for which a result could not be determined due to mixed-field reaction were excluded. Error bars are the standard errors of the mean (SEM), ****P<0.0001, ns=not significant (Mann-Whitney U test).

FIG. 4 continued (c,d) Reactivity of AmGH36A-treated A₁ RBCs as compared to one-pot-treated A₁ RBCs using a AmGH36A, AmGH95B and AmGH35A cocktail with group AB (n=11) and group B (n=12) plasmas tested with both the secretor A₁ RBCs (c) and non-secretor A₁ RBCs (d). Of the AB plasmas, which are negative with the native A₁ RBCs used here (data not shown), one reacts with the AmGH36A-treated RBCs from secretor and one reacts with the triple treated secretor and non-secretor RBCs. For secretor RBCs, the treatment with AmGH36A alone abolished positive crossmatch reactions for 5 out of 12 type B donor plasmas, whereas the treatment with the three enzyme cocktail abolished positive cross-matches in 9 out of 12 plasmas. The remaining positive crossmatches were weakened (≤1+) for the enzyme cocktail treatment. Similar results were also shown for the enzyme cocktail treatment of non-secretor RBCs and B plasmas. *Denotes crossmatches were fibrinogen where noted.

FIG. 5a,b Crossmatch test of B secretor or non-secretor RBCs treated with AmGH110A, compared to one-pot treatments with either AmGH110A and AmGH20A, or AmGH110A and AmGH20C. Reactivity was graded as positive, 1+ to 4+[where the weakest is denoted (1+) and the strongest has the highest number] and negative, −[where suggestion of a reaction is denoted (−)]. a) The distribution of the O plasma (n=100) reactivities with the treated B RBCs as well as comparison between the different treatments, b) a graph comparing the mean crossmatch reactivity values of (a), cross-matches of native RBCs with ten of the O plasmas are included for comparison. Error bars are SEM and *P<0.05, ***P<0.001, ns=not significant (Mann-Whitney U test). E.g. For the secretor RBCs the AmGH110A treatment alone resulted in abolished positive crossmatches by 79%, as compared to untreated cells. By comparison, the conversion with the AmGH110A and AmGH20A abolished positive crossmatches in 91% as compared to untreated cells, which provides strong evidence that removal of the B antigen alone is necessary but not sufficient for compatibility and that ExtB or GalNAc B antigen is important to compatibility of the converted RBCs.

FIG. 5 continued (c,d) The crossmatch reactivity of secretor B RBCs (native and enzyme-treated) with c) AB plasma (n=11) and d) A plasma (n=12), showing that crossmatches of group B RBCs with group A plasmas were abolished after treatment of the RBCs with the AmGH110A and AmGH20A, or AmGH110A and AmGH20C enzyme combinations.

FIG. 6. A phylogenetic tree generated from blast searches against the NCBI non-redundant database (https://blast.ncbi.nlm.nih.gov/Blast.cgi?PROGRAM=blastp&PAGE_TYPE=BlastSearch& LINK_LOC=blasthome) under default settings using the catalytic domains of each of the GH20 enzymes from A. muciniphila (SEQ ID NO. 2, 4, 6, 18, 20, 22, 24, 26, 28, 30, 32), and including the previously characterized GH20 enzymes having β-N-acetyl-6-sulfo-glucosaminidase activity, BbhII (BAI94823.1) and Btheta7330_03706 (ALJ43236.1), and endo-β-N-acetylglucosaminidase activity BF3095 (CAH08790.1), and lacto-N-triose activity, BbhI (BAI94822.1) as query sequences for 15 total blastp searches. The catalytic domain of each query sequence, were assigned as the region that corresponds to amino acid residues 164-552 of AmGH20A SEQ ID NO.: 2 in a multiple sequence alignment performed by MAFFT v. 7.520, accessed on the MAFFT webserver (https://mafft.cbrc.jp/alignment/sever/).

Each blast was performed using default setting and was set to retrieve a maximum of 500 hits with a sequence coverage of >90% and sequence identity of 30-95% to the query sequence to remove fragments and exclude redundant sequences that share >95% sequence identity and to exclude distant sequences with <30% sequence identity. Sequences were retrieved and trimmed to include only catalytic domains by multiple sequence alignment in MAFFT (see above), by manually removing excess sequence on either side of the respective query sequence catalytic domain in the multiple sequence alignment using the multiple sequence alignment viewing program, JalView v. 1.8.3. The sequences from the 15 blast searches were combined and further filtered for redundancy using the CD-HIT tool on a local server downloaded from github (https://github.com/weizhongli/cdhit/releases/) with a 95% sequence identity cutoff, assuming these sequences were functionally redundant. This resulted in the alignment of 1350 sequences, which were used to generate the phylogenetic tree using the MAFFT neighbor-joining algorithm with bootstraps performed with 1000 iterations. Previously characterized enzymes are indicated with their enzyme name, Genbank accession and substrates, where enzyme reactivity has been demonstrated. The different clusters are denoted with Roman numerals.

FIG. 7. A cartoon showing domain organization and sequence divergence of A. muciniphila enzyme candidates for conversion of the extended GalNAc-B antigen. a) Domain organization of the candidate GH20 enzymes from A. muciniphila, where AmGH20A, AmGH20B, AmGH20F, AmGH20G, AmGH20J contain one or more unannotated putative domains at the C-terminus, while AmGH20J has an additional unannotated N-terminal patch. Domain annotations are based on the CAZy, DBcan, and Interpro databases. b) Amino acid sequence identity (%) matrix between the AmGH20 enzymes in a) based on alignment of respective catalytic domains.

FIG. 8. Structural comparison of AmGH20A and AmGH20C showing that both enzymes possess several positively charged amino acid residues flanking the active site. The distances (Å) from the residues to the active site-bound GalNAc are shown.

FIG. 9. Structural comparison of AmGH20A and AmGH20C showing a different arrangement of two loops flanking the active site in the two enzymes.

FIG. 10 a-f The evaluation of extended GalNAc—B type enzyme candidates towards B type RBCs by immunostaining (using human eluate of anti-GalNAc-B with GAH-IgG/GAH-IgM-PE mix as primary and secondary Abs respectively) and flow cytometry. The histograms of the B RBCs (solid grey histograms) are compared to the negative control of O RBC (dotted black line histograms). a) Activity screening of eleven A. muciniphila GH20 enzymes and a single AmGH123 on the GalNAc-B antigen on B type RBCs from two donors. The screening was performed using 1 μM of each enzyme at standard conditions (30 min, room temperature in conversion buffer, 38% hematocrit) and the data are shown as the Median Fluorescence Intensity (MFI) of the PE fluorescence signal, with the open circles representing the data from two RBC donors. b) Representative native type B RBC controls with no enzyme treatment. c), d) or e) Treatment of cells in b) with AmGH20A, AmGH20C and AmGH20I, respectively. f) An overview of the data in b-e shown as the MFI of the PE signal. The open circles represent data from three RBC donors (n=3).

FIG. 10 continued g-m Analyses of the candidate enzymes that are most active on the extended GalNAc-B antigen on B RBCs. g), h) and i) Evaluation of the conversion efficacy of AmGH20A, AmGH20C, and AmGH20I at different enzyme concentrations following incubations with B type RBCs in the presence of 0.5 μM AmGH110A at standard conditions. j) and (k) are additional comparisons of the efficacy of AmGH20A and AmGH20C, respectively at standard conditions, but without treatment with AmGH110A. l) and m) show conversion of the GalNAc B epitope on RBC in conversion buffer, which is characterized by maintaining low ionic strength, isotonic and neutral pH conditions, and in phosphate buffered saline (βBS, differing from the conversion buffer by having a physiological ionic strength), respectively, using AmGH20A (0.2 μM) or AmGH20C (0.1 μM).

FIG. 11. Phylogenetic tree of GH110 that harbors B type antigen active enzymes. Previously characterized enzymes are indicated with enzyme name, Genbank accession and substrates, where enzyme reactivity has been demonstrated. The B converting α-galactosidase, AmGH110A, populates cluster II. The phylogenetic analysis is based on a multiple sequence alignment of the catalytic domains of sequences downloaded from the CAZy database (April 2023) (https://www.crazy.org/GH110.html). A total of 948 sequences were retrieved, and the catalytic domain of the sequences were assigned as the region corresponding to amino acid residues 213-795 of AmGH110A (SEQ ID NO. 8) in a multiple sequence alignment. Sequences were manually trimmed to include only the catalytic domain using the multiple sequence alignment viewing program, JalView v. 1.8.3. The sequences were filtered using the MaxAlign tool at default settings, accessed using the DTU HealthTech server (https://services.healthtech.dtu.dk/services/MaxAlign-1.2/) to maximize the size of gap-free columns in the alignment, resulting in a total of 748 orthologue sequences which were aligned to generate the phylogenetic tree using the MAFFT neighbor-joining algorithm with bootstraps performed with 1000 iterations, accessed on the MAFFT webserver (https://maffft.cbrc.jp/alignment/server/). The different clusters are denoted with Roman numerals.

FIG. 12. Domain organization and sequence divergence of A. muciniphila enzyme candidates for B type antigen conversion as compared to previously described enzymes shown to be active on RBCs from glycoside hydrolase (GH) families belonging to: a) GH110, c) GH27, and e) GH36. Amino acid sequence identity (%) matrices for said enzymes are shown in: b) GH110, d) GH27 and f) GH36, based on alignment of their respective catalytic domains. Domain annotations are based on the CAZy, DBcan, and Interpro databases.

FIG. 13. Overall structure and domain organization of AmGH110A. The enzyme comprises a β-helix catalytic domain, two β-barrel domains (I and II) as well as an N-terminal putative carbohydrate binding module of CBM51. The solid spheres are two putative Ca²⁺ ions, based on coordination type and geometry.

FIG. 14. The evaluation of enzyme candidates towards B type RBCs using immunostaining and flow cytometry. Representative flow cytometry histograms of anti-B (clone 9621A8)/anti-RAMκ-phycoerythrin (PE)-stained B RBCs (solid grey histograms) and native O RBCs as negative control (dotted black line histograms). a) Representative native RBC controls. b), c), d) or (e) are the RBCs in a) after treatment with 1 μM of AmGH110A, AmGH36A, AmGH36C or AmGH27, respectively at standard conditions. f) Overview of the data in a-e with the bars displaying the mean of the median fluorescence intensity (MFI) of the PE signal of B RBC aliquots from three donors (n=3) and the open circles represent the MFI from each donor. g) Untreated native type B RBC controls. h) The RBCs in g) after treatment with 0.12 μM AmGH110A for 60 min at room temperature in the conversion buffer. i) Treatment as in h), but carried out in phosphate buffered saline (βBS). J) Treatment of B type RBCs from a single donor at different AmGH110 concentrations and incubation times at otherwise standard conditions. K) Treatment with three different AmGH110 concentrations carried out on RBCs from three donors, at standard conditions. For both j and k the dotted line in each graph is the MFI level of the O type RBCs as negative controls.

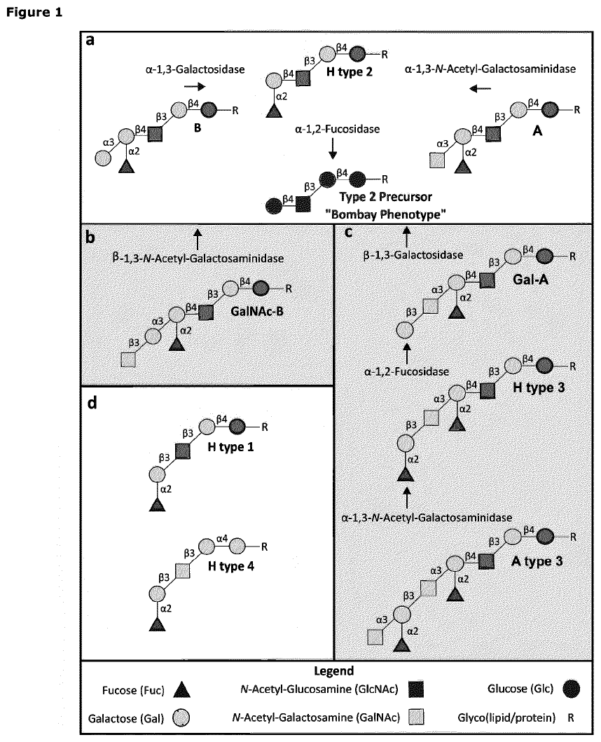

FIG. 15. Image of thin layer chromatograms [TLC](panels a-f) showing the time course of hydrolysis of 2 mM B type 2 antigen (type 2 chain, see FIG. 1a) hexaose substrate at 37° C. by 1 μM of each of the A. muciniphila enzyme candidates assayed in 20 mM sodium phosphate, 150 mM NaCl, 0.005% (v/v) Triton X-100, pH 7.0 (based on independent duplicate TLC data). The graph (panel h) shows the initial rate of hydrolysis of 2 mM B antigen by AmGH110A, measured from the time-course release of galactose from B type 2 hexaose at 20 nM at 37° C. (panel g) using a galactose standard curve generated from HPAEC-PAD analyses based on the area under the peak at 6 minutes retention time.

FIG. 16. Structural signatures of AmGH110A and AmGH110B. a) and b) depict unique signatures in 5 loops that distinguish the active site face of the B type converting AmGH110A from the non-converting AmGH110B, respectively.

FIG. 17. Sequence Logos that depict the motifs of Loop 1 and loop 2 in different GH110 phylogenetic clusters.

FIG. 18. Sequence Logos that depict the motifs of Loop 3-5 in different GH110 phylogenetic clusters.

FIG. 19. Phylogenetic clustering of GH36 enzymes of different specificities. The α-N-acetylgalactosaminidases populate clusters I-III, whereas the remaining specificities populate a different clade with clusters IV-VII. The phylogenetic analysis is based on a multi-sequence alignment of sequences retrieved by a blastp search against the NCBI non-redundant database (https://blast.ncbi.nlm.nih.gov/Blast.cgi?PROGRAM=blastp&PAGE_TYPE=BlastSearch& LINK_LOC=blasthome) using each of the catalytic domains of AmGH36A, AmGH36C, AagA (Clostridium perfringens), MelA (Lactobacillus acidophilus), and FpGalNAse as query. The catalytic domain of AmGH36A (residues 385-749 of SEQ ID NO.:10) was used to assign the regions corresponding to the catalytic domains of the above GH36 enzymes prior to the blast search against the NCBI non-redundant database. The blast was performed using default setting to retrieve target sequences with a sequence coverage of >90% and sequence identity 25-95% to the query to reduce redundancy and exclude distant sequences with <25% sequence identity. A total of 5753 sequences were retrieved, and were trimmed to include only catalytic domains by multiple sequence alignment, manually removing excess sequence on either side of the respective query sequence catalytic domain in the alignment, using the multiple sequence alignment viewing program, JalView v. 1.8.3. Sequences from all blast results were combined and further filtered for redundancy using the CD-HIT tool on a local server downloaded from github (https://github.com/weizhongli/cdhit/releases) with a 95% sequence identity cutoff to remove sequences assumed to be functionally redundant. This resulted in the alignment of 1726 sequences to generate the phylogenetic tree using the MAFFT neighbor-joining algorithm with bootstraps performed with 1000 iterations, accessed on the MAFFT webserver (https://mafft.cbrc.jp/alignment/server/). The different clusters are denoted with Roman numerals.

FIG. 20. Phylogenetic clustering of GH109 enzymes from the CAZy database (Drula E, 2022 (http://www.cazy.orq/)). Previously characterized enzymes are indicated with enzyme name, Genbank accession and substrates, where enzyme activity has been demonstrated. The A antigen converting α-N-acetylgalactosaminidase, AmGH109B, populates cluster II. The phylogenetic analysis is based on the multi-sequence alignment of GH109 catalytic domains that were retrieved from the CAZy database (http://www.cazy.org/GH109.html). 2114 sequences were retrieved, and the catalytic domain of the sequences were assigned as the region corresponding to amino acid residues 27-481 of AmGH109BA (SEQ ID NO. 12) in a multiple sequence alignment. Sequences were manually trimmed to include only the catalytic domain using the multiple sequence alignment viewing program, JalView v. 1.8.3, and filtered for redundancy using the CD-HIT tool on a local server downloaded from github (https://github.com/weizhongli/cdhit/releases) with a 95% sequence identity cut-off. This resulted in the alignment of 646 sequences that were used to compute the phylogenetic tree using the MAFFT v. 7.520 neighbor-joining algorithm with bootstraps performed with 1000 iterations, accessed on the MAFFT webserver (https://mafft.cbrc.jp/alignment/server/). The different clusters are denoted with Roman numerals.

FIG. 21. a) A cartoon showing domain organization and sequence divergence of A. muciniphila GH109 enzyme candidates, and the previously characterized NagA. b) Amino acid sequence identity (%) matrix of AmGH109 enzymes and NagA based on alignment of catalytic domains wherein amino acid residues 27-481 of SEQ ID NO.: 12, comprise the catalytic domain of AmGH109B. Domain organization of the enzymes, based on the CAZy, DBcan (Yin Y, 2012 (https://bcb.unl.edu/dbCAN/)) and Interpro (Paysan-Lafosse T, 2022 (https://www.ebi.ac.uk/interpro/)) databases.

FIG. 22 a-j. The evaluation of the α-N-acetylgalactosaminidase candidates in the conversion of A antigens on group A RBCs using immunostaining with an anti-A antibody, ES-15, followed by RAMκ-phycoerythrin(PE) and flow cytometry. The flow cytometry histograms of the group A RBCs (solid grey histogram) as compared to the group O RBC negative control (dotted black line histograms). a untreated RBCs of the common A₁ phenotype. c, e, and g RBCs in a) after treatment with 1 μM of AmGH109A, AmGH109B or AmGH36A, respectively in the conversion buffer. b representative histograms of RBCs of the less common A₂ phenotype (which has a lower density of A type antigens on the surface of RBCs. d, f) and h RBCs in b) after treatment with 1 μM of AmGH109A, AmGH109B or AmGH36A. i and j Overview of the data shown in panels a-h, which is represented as the MFI of RBCs from three different donors (n=3) of each phenotype, with the data from each donor shown as an open circle.

FIG. 22 continued (k-r) Analyses of the activity of α-N-acetylgalactosaminidase candidates acting on the A and A type, 3 antigens on group A RBCs in different buffers and at different enzyme concentrations. k flow cytometry histograms of A-stained native RBCs as controls as compared to: I) and m) after treatment with AmGH109B (8 μM) in conversion buffer and phosphate buffered saline (βBS), respectively. n and o the same as l) and m), but the treatment is conducted with AmGH36A (1.1 μM). p the A antigen levels of A₁ phenotype RBCs after different incubation times at different AmGH36A concentrations. q) and r) A and and A type 3 antigens' level (immunostained with an anti-A type III Ab (TH1) and RAMκ:PE) of A₁ phenotype RBCs from three donors (n=3) after incubation with different AmGH36A concentrations for 30 min at room temperature.

FIG. 23. AlfaFold model of the A type antigen active AmGH36A structure. Besides the (β/α)₈ catalytic domain and the C- and N-terminal β-sheet domains, the model reveals the presence of an additional β-sandwich CBM-like domain that is positioned above the active site in a Cobra attack pose architecture. a) Side view of the model, showing the spatial arrangement of domains. b) A top view along the axis of the (β/α)₈ barrel.

FIG. 24: Thin layer chromatography (TLC) analysis of the activity of A. muciniphila enzyme candidates against the A type 2 hexaose. a) Time course of hydrolysis of the terminal non-reducing GalNAc from the A type 2 hexaose by AmGH109B. b) As in a), but using the AmGH109A candidate. c) As in a) but using the AmGH36A candidate. All the reactions were performed using 1 μM of each enzyme and 2 mM of each A antigen oligosaccharide in 20 mM sodium phosphate, 150 mM NaCl, 0.005% (v/v) Triton X-100, pH 7.0 at 37° C.

FIG. 25. Potential structural determinants of the N-acetylgalactosaminidase activity within GH36. a) AlfaFold model of AmGH36A with a superimposed GalNAc in the active site. b) The residues that form the N-acetyl accommodation pocket at the C2 position in N-acetylgalactosaminidases as exemplified by AmGH36A. c) and d) The residues that block the accommodation of the C2 N-acetyl unit in the α-galactosidase, AmGH36C, and the α-galactosaminidase, FpGalNase, respectively.

FIG. 26. Structural signatures of AmGH36A as compared to the α-galactosidase AmGH36C. a) Two active site flanking loops that offer potential signatures for the A type converting AmGH36A. b) The same loops in a), in the α-galactosidase AmGH36C.

FIG. 27. Loop sequence motif signatures of different phylogenetic clusters within GH36. a) and b) Sequence motifs of the surface loop 1 and 2, respectively, in different GH36 clusters.

FIG. 28. Phylogenetic clustering of characterized GH95 α-1,2-fucosidases. The phylogenetic analysis is based on the multi-sequence alignment of sequences that were retrieved by blast searches using the sequences of AmGH95A residues 26-763 (SEQ ID NO. 39) and AmGH95B residues 28-770 (SEQ ID NO. 13) against 7950 (meta)genomes from the human gut microbiota database (Almeida A, 2021) in November 2021. The redundancy of retrieved sequences was reduced using the CD-HIT tool on a local server downloaded from github (https://github.com/weizhongli/cdhit/releases), with a 95% sequence identity cut-off. This resulted in the alignment of 548 sequences that were used to compute the phylogenetic tree using the MAFFT neighbor-joining algorithm with bootstraps performed with 1000 iterations, accessed on the MAFFT webserver (https://mafft.cbrc.jp/alignment/server/). The different clusters are denoted with Roman numerals.

FIG. 29. Overall structure of AmGH95B in complex with fucose. a) Side view orthogonal to the active site showing the flat topology of the enzyme active site. b) Top view along an axis orthogonal to the (β/α)₈ barrel.

FIG. 30 a-i The evaluation of α-1,2-fucosidase candidates that target H antigens on RBCs. The conversion is analysed by immunostaining (by monoclonal BRIC231/RAMκ-phycoerythrin(PE) staining) and flow cytometry. a) Representative histograms of untreated O RBCs. b), c), and d) The flow cytometry histograms from group O RBCs post incubations with the previously characterized Roseburia inulinivorans fucosidase RiFuc95 [SEQ ID NO.: 45], AmGH95A and AmGH95B, each at 1.2 μM for 60 min in conversion buffer. e) Overview of the data in b-d, which is represented as the MFI from a single donor (n=1). f), g), and h) the corresponding reactions to panels b-d, performed in PBS buffer. i) Overview of the data in f-h, which is represented as the MFI from a single donor (n=1).

FIG. 30 continued (j-t) The evaluation of the top α-1,2-fucosidase candidate in a sequential reaction with the top candidate enzyme active on the A antigen in the conversion of A and extended A antigens on RBCs. j-m) Representative flow cytometry histograms of A₁ phenotype secretor RBCs (solid grey histogram) stained with an anti-A antibody (ES-15 followed by RAMκ-PE staining), anti-H (BRIC231/RAMκ-PE), anti-H type 3 (HH14/RAMκ-PE) and anti-Gal-A (3C9/RAMκ-PE), antibodies, respectively. The dotted black line histogram is a negative control (Bombay RBCs for H staining and O RBCs for the others) for comparison. n-q) the corresponding histograms to panels j-m post treatment with 1 μM AmGH36A. r), s) and t) the corresponding histograms to panels o-q, post treatment with 1 μM AmGH95B.

FIG. 30 continued (u) One pot analysis of different concentrations of the α-1,2-fucosidase and α-N-acetylgalactosaminidase that are most active in the conversion of A and H antigens, respectively, on group A RBCs. u) The MFI on native A₁ RBCs (n=3), treated with AmGH36A combined with increasing concentrations of AmGH95A for 30 min at room temperature in conversion buffer.

FIG. 31: Thin layer chromatography analysis of the activity of α-1,2-fucosidase candidates from A. muciniphila and the previously characterized counterpart from Roseburia intestinalis [SEQ ID NO.: 45] against the H type 1 pentaose and H type 2 triose. a), b) and c) are the time course defucosylation of the H type 1 pentaose with AmGH95B, AmGH95A and RiFuc95, respectively. d), e) and f) the reactions of the same enzymes as in a), but using the H type 2 triose as a substrate. All the reactions were performed using 1 μM of each enzyme and 2 mM of each B antigen oligosaccharide in 20 mM sodium phosphate, 150 mM NaCl, 0.005% (v/v) Triton X-100, pH 7.0 at 37° C.

FIG. 32. Structural comparison of AmGH95B and AmGH95A. a) Divergent active site flanking loops, which correlate to the activity of AmGH95B on RBCs as opposed to b) AmGH95A corresponding loop regions, which results in a different active site topology between the two enzymes, with a shallowed flat active site in AmGH95B.

FIG. 33. Sequence logos of GH95 loop 4 from different phylogenetic clusters, showing the emergence of unique signatures for GH95 enzymes of cluster III.

FIG. 34. Phylogenetic tree of GH35 enzymes with different specificities. The GalA antigen active GH35 enzyme, AmGH35A falls into cluster IV, while other GH35 enzymes populate the remaining clusters (I-III) and (V-VIII). The phylogenetic analysis is based on the multi-sequences that were downloaded from the CAZy database (April 2023) (http://www.cazy.org/GH35.html). 8380 sequences were retrieved, and the catalytic domain of the sequences were assigned as the region corresponding to amino acid residues 26-379 of AmGH35A (SEQ ID NO. 16) in a multiple sequence alignment. Sequences were manually trimmed to include only the catalytic domain using the multiple sequence alignment viewing program, JalView v. 1.8.3. The sequences were filtered for redundancy using the CD-HIT tool on a local server downloaded from github (https://github.com/weizhongli/cdhit/releases), with a 95% sequence identity cut-off, and subsequently filtered using MaxAlign at default settings, accessed using the DTU Health Tech website (https://services.healthtech.dtu.dk/services/MaxAlign-1.2/) to maximize the size of gap-free columns in the alignment. This resulted in the alignment of 1763 sequences that were used to compute the phylogenetic tree using the MAFFT v. 7.520 neighbor-joining algorithm with bootstraps performed with 1000 iterations, accessed on the MAFFT webserver (https://mafft.cbrc.jp/alignment/server/). The different clusters are denoted with Roman numerals.

FIG. 35. A cartoon showing domain organization and sequence divergence of A. muciniphila enzyme candidates for conversion of the extended Gal-A antigens. a) Domain organization of A. muciniphila GH35 enzyme candidates for the conversion of the extended Gal-A antigen. b) Amino acid sequence identity (%) between the catalytic domain of the two GH35 candidates.

FIG. 36. Image of thin layer chromatograms [TLC] showing the activity profiles of the A. muciniphila GH35 β-galactosidase candidates against the Gal-A epitope. a) The activity profiles of AmGH35A candidates towards β-galactoside disaccharides, including the T-antigen disaccharide (Galβ1,3GalNAc) that resembles the same motif of the Gal-A extended epitope (see FIG. 1c). b) The same as a), but using AmGH35A. Reactions were performed using 1 μM of each enzyme and 2 mM of each oligosaccharide in 20 mM sodium phosphate, 150 mM NaCl, 0.005% (v/v) Triton X-100, pH 7.0 at 37° C.

FIG. 37. The evaluation β-1,3-galactosidase candidates that target the extended Gal-A on A₁ RBCs by immunostaining with an anti-Gal-A monoclonal antibody (3C9/RAMκ-phycoerythrin(PE)) and flow cytometry. Representative flow cytometry histograms (solid grey histogram) of the Gal-A expression of A₁ RBCs from a single donor (n=1) are shown and histograms of the negative controls, O RBCs, are shown with a dotted black line histograms. a) Untreated A₁ RBCs. b) and c) RBCs as in a) post treatment with 1 μM of AmGH35A and AmGH35B, respectively in standard conditions. d) The MFI of histograms shown in (a-c), data of one donor. e) The MFI of A₁ RBCs from three donors native RBCs or post treatment with AmGH36A, or combined treatments with AmGH36A and AmGH95B, or AmGH36A, AmGH95B and increasing concentration of AmGH35A.

FIG. 38. Histogram showing Cy5 total fluorescence detected in a solution of each Cy5-labelled enzyme in conversion buffer (total); and by RBCs after incubation in the respective conversion buffer (RBCs), and by said RBCs following four consecutive washing steps with PBS (W1, W2, W3 and W4). An α-1,6-glucosidase (LaGH13_31), which based on its activity was not expected to specifically interact with RBCs, was used as a negative control for both the A and B RBCs. The fluorescence associated with the RBCs was estimated by subtracting the fluorescence in each supernatant after pelleting the RBCs from the total initial fluorescence (total). The data are reported as means of four independent experiments and error bars are SD, ****P<0.0001, ***P<0.001, **p<0.01, *P<0.05, N. S.=not significant (paired sample t-test). The residual fluorescence % measured after the last wash is shown.

FIG. 39. Flow cytometry detection of residual Cy5-labelled enzymes on RBCs following incubation in conversion buffer comprising Cy5-labelled A (AmGH36A Cy5) or B (AmGH110A Cy5) enzymes or a negative control (LaGH13_31) enzyme, and washing with PBS buffer under conditions mimicking the standard conversion ad washing protocol used herein. The conversion buffer for B RBCs comprised either Cy5-labelled AmGH110A enzyme alone or in the one-pot B-cocktail. The conversion buffer for A RBCs comprised either Cy5-labelled AmGH36A enzyme alone or in the one-pot A-cocktail. The indicated % is the fraction of the cell population that shows an associated Cy5-signal. This data are based on a single experiment.

### ABBREVIATIONS, TERMS AND DEFINITIONS

### Ab(s): Antibody/Antibodies

A or B or AB red blood cells: are A or B or AB RBCs that are derived from corresponding phenotype donor RBCs or corresponding phenotype RBCs derived from hematopoietic stem and progenitor cells (Giarratana et al 2011; Trakarnsanga et al., 2017) Amino acid sequence identity: The term “sequence identity” as used herein, indicates a quantitative measure of the degree of similarity between two amino acid sequences of essentially equal length. The two sequences to be compared must be aligned to give a best possible fit, by means of the insertion of gaps or alternatively, truncation at the ends of the protein sequences. The sequence identity can be calculated as ((Nref-Ndif)100)/(Nref), wherein Ndif is the total number of non-identical residues in the two sequences when aligned and wherein Nref is the number of residues in one of the sequences. Sequence identity calculations are preferably automated using the BLAST program e.g. the BLASTP program (βearson W. R and D. J. Lipman (1988)) (www.ncbi.nlm.nih.gov/cgi-bin/BLAST). Sequence alignment may be performed using program MAFFT24 (Multiple Alignment using Fast Fourier Transform; Katoh et al 2019) using default parameters (SCORING MATRIX: blosum62, gap opening penalty: 1.53, gap extension penalty 0.123).

Preferably, the numbers of substitutions, insertions, additions or deletions of one or more amino acid residues in the polypeptide as compared to its comparator polypeptide is limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions. Preferably the substitutions are conservative amino acid substitutions: limited to exchanges within members of group 1: Glycine, Alanine, Valine, Leucine, Isoleucine; group 2: Serine, Cysteine, Selenocysteine, Threonine, Methionine; group 3: Proline; group 4: Phenylalanine, Tyrosine, Tryptophan; Group 5: Aspartate, Glutamate, Asparagine, Glutamine; Group 6: Histidine. Lysine, Arginine.

Cluster: is a group of sequences that based on their similarity and ultimately their phylogenetic relationship are found to fall within a subclade defined by a phylogenetic tree. Methods for assigning any one of the converting enzymes according to the present invention to its respective cluster of its respective phylogenetic tree is disclosed in the figure legends provided for each of the phylogenetic trees illustrated herein.

Converting enzyme: An enzyme that alone or together with other converting enzymes converts A type or B type antigens, or extended forms thereof, to a form that has substantially reduced levels of these antigens, which would otherwise, if not depleted, result in a positive and often severe (≥3+) crossmatch with ABO-incompatible groups.

Conversion buffer: is characterized by low ionic strength, isotonic and neutral pH (e.g. 200 mM glycine, 3 mM NaCl, pH 6.8).

ExtB: is an extended GalNAc-B antigen.

Isotonic solution: is a solution that has the same or similar solute concentration and water concentration compared to body fluids. This can be achieved for example using buffered phosphate saline or other buffer-salt combinations, (e.g., conversion buffer). The use of such buffers is crucial for the stability of RBCs, since a hypertonic or hypotonic solution will compromise the physical stability and viability of RBCs.

Low ionic strength: is a solution that has significantly lower ionic strength than an isotonic solution and can be used to increase the interaction between an enzyme and the cell surface, in this case various exoglycosidases and the red blood cell surface. Any solution with a lower ionic strength than isotonic, can be considered a Low Ionic Strength Solution (LISS). However, the most suitable ionic molarity (e.g. NaCl) for enzyme conversion for the exoglycosidases used herein is preferably approximately 3 mM, but conversion occurs suboptimally also at 3-15 mM or even up to 30 mM. The glycine content has to be adjusted accordingly, i.e. decreased glycine if the ionic strength increases.

Neutral pH: is a pH that is compatible with maintaining functional RBCs, preferably from 6.5 to 7.5, more preferably from 6.8 to 7.4.

PBS: is phosphate buffered saline having a physiological ionic strength.

Room temperature optimized for laboratory work is 20-25° c.

### Detailed Description of the Invention

The present invention is based on the observation that ABO antigens displayed on the surface of RBCs do not solely comprise the well characterized A type and B type antigens, but also include extended structures with additional saccharide units bound to the terminal non-reducing end of A type and B type antigens. The precise structure of three extended antigens on the surface of group A RBCs has been reported, and recently a single extension of the B antigen has been discovered. However, the significance of the extended structures for transfusion compatibility has not been addressed to date. Hence the provision of enzymes according to the present invention, that act on these antigens, provides a potent enzyme cocktail needed for the conversion of A or B (or AB) RBCs including all known extended epitopes to blood of universal type. Hence the present invention provides a source of enzyme-converted ABO-compatible RBCs to meet the needs for future blood transfusions that can be produced from A or B (or AB) blood using the enzyme cocktail of the present invention.

### I: Enzyme-Converted ABO-Compatible RBCs

In a first embodiment, the invention provides a population of enzyme-converted RBCs having no detectable A-antigens, B-antigens, extended A- and extended B-antigens as determined by serological typing or, wherein said enzyme-converted ABO-compatible RBCs are obtained by enzymatic removal of A-, B-antigens and extended A- and extended B-antigens from A or B (or AB) RBCs.

Enzyme-converted, ABO-compatible RBCs (RBCs), as defined herein are cells that have no detectable A-antigens, B-antigens and extended A- and extended B-antigens, which is generally expected to significantly reduce or abolish cross-match-reactivity compared to the A or B (or AB) RBCs from which they were derived. Alternatively, the level of said detectable A-antigens, B-antigens and extended A- and extended B-antigens on said RBCs is significantly reduced relative to corresponding untreated A or B (or AB) RBCs, for example the level is reduced by at least 90, 95, 98, 99 or 100% as measured by antibody staining and flow cytometry, as described herein.

Enzyme-converted RBCs are those, where the depletion of the extended and the A type or B type antigens can be demonstrated by serological typing. Preferably serological typing is performed by using monoclonal antibodies and immunostaining followed by flow cytometry, as described herein. For immunodetection of B antigens, RBCs are incubated with monoclonal anti-B (clone 9621A8, Diagast, Loos France) followed by a Phycoerythrin (PE) conjugated secondary rat anti-mouse antibody (RAM-κ:PE, BD Biosciences San Jose, CA, USA) using a protocol that is optimized to detect low antigen amounts (Hult and Olsson 2010). The RBCs PE-signal is recorded on a BD FACS Canto II and analyzed in the software FCS express v. 6. The extended GalNAc-B antigen can be detected using monoclonal antibodies generated against the GalNAc-B antigen, or by using polyclonal antibodies obtained by adsorbing the naturally-occurring anti-GalNAc-B antibodies in human plasma of a blood group B or AB P1k donor onto B RBCs and subsequent elution of these with heat (56° C.) (Judd (1999)). Thereafter this eluate is used as a primary antibody followed by a mix of secondary goat anti-human-IgG: PE and goat anti-human-IgM:PE (both from Jackson Immunoresearch Europe, Cambridge, UK).

The highly sensitive anti-A (clone ES-15 from Quotient, Eysins, Switzerland) recognizes small amounts of A antigen on RBCs. Anti-H (clone BRIC231) from the International Blood Group Reference Laboratory (IBGRL research products, Bristol, UK) is used for total H antigen immunodetection. The detection of the extended A type 3 (Clausen 1985), H type 3 (Clausen 1986; I and II) and Gal-A (Clausen 1988) can be performed using monoclonal antibodies raised against each of these antigens, e.g. TH-1, HH14 and 3C9, respectively. The detection of the extended A type 3 and the A antigens can be achieved using the RAM-K: PE secondary antibody.

Furthermore, traditional serological typing on gel-cards (Bio-Rad, Hercules, CA, US) or in tube can also be conducted using Anti-A (Seraclone/Bio Rad), anti-B (Diagast) and anti-H (lectin of Ulex Europaeus, Immucor, Norcross, GA, USA).

The efficacy of enzymatic treatment according to the present invention for the removal A-antigens, B-antigens and extended A- and extended B-antigens from A or B (or AB) RBCs can be determined by its improved compatibility (against a cohort of group O plasmas) as compared to:
i. a corresponding untreated A, B or AB RBCs, or
ii. enzyme-converted A, B or AB RBCs obtained from corresponding untreated A, B or AB RBCs by enzymatic removal of only A-, B-antigens,
wherein said compatibility is tested against a cohort of group O plasmas.

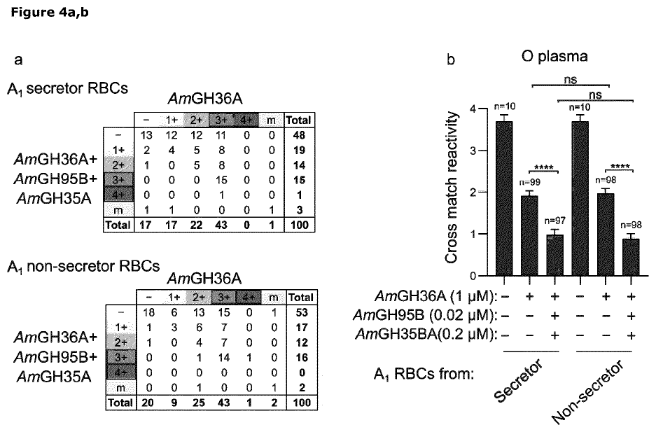

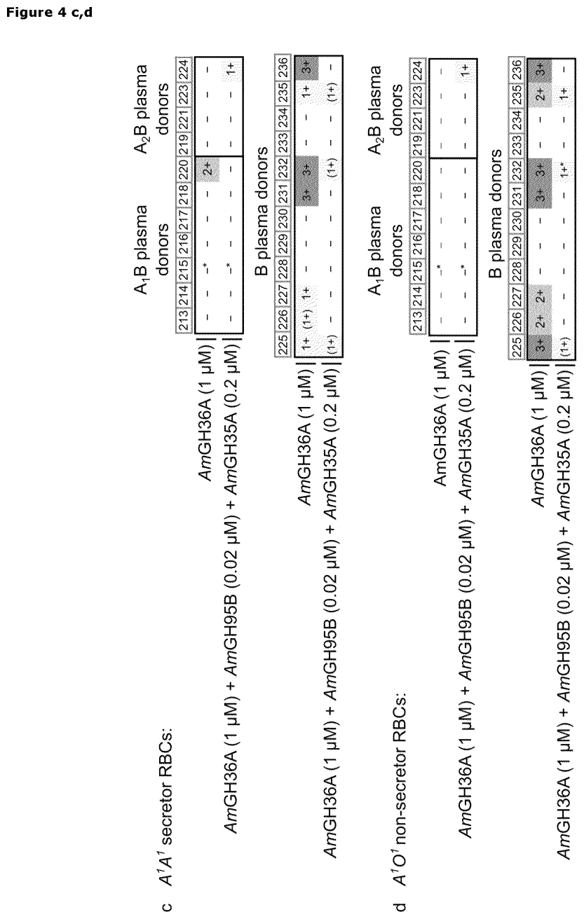

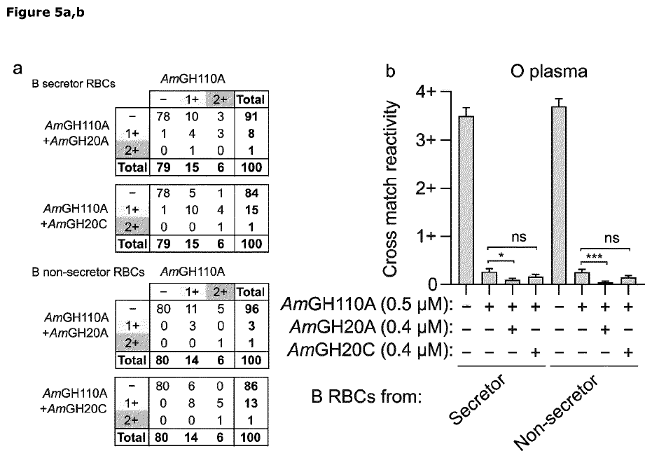

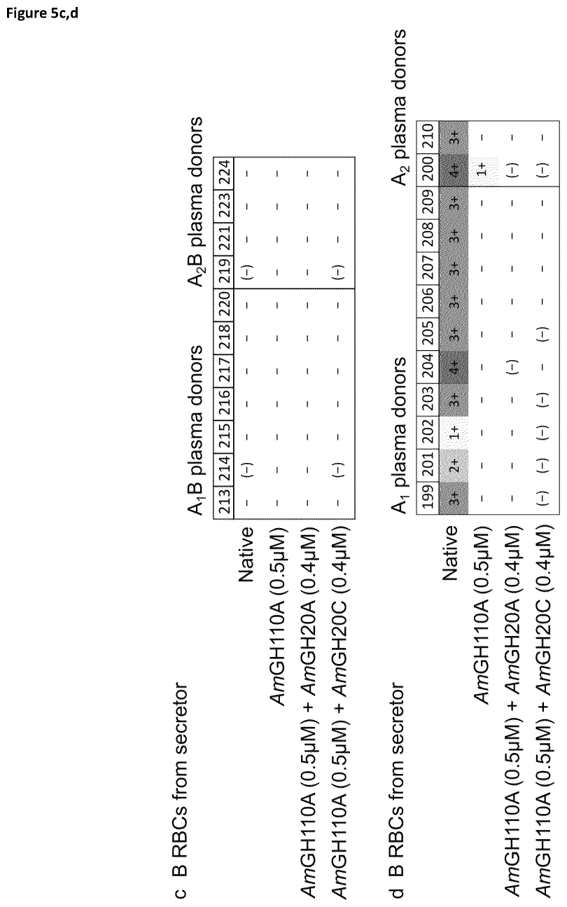

The RBCs are ABO-compatible such that the risk of fatal reactions with plasma from patients having any of the main groups (A, B, O, and AB) is significantly reduced as compared to antigen A-converted and/or antigen B-converted RBCs, such that the converted RBCs can be used for blood transfusion to a greater number of patients in need (e.g. FIGS. 4 and 5). As demonstrated herein, in a test population of enzyme-converted B RBCs (n=100) obtained using enzymes according to the present invention when tested against a large cohort of group O plasmas showed a total percent of compatibility of 91% (measured as negative crossmatch reactivity; example 2vi).

ABO-compatible RBCs are defined herein as being enzyme-converted because they are derived from A, B or AB RBCs from which any A-antigen, B-antigen and known extended A- and extended B-antigens that was present on the RBCs have been removed enzymatically. The structure of an extended B antigen is a GalNAc-extended B antigen and the structures of extended A antigens comprise A type 3-, H type 3- and Gal-A-extended antigens (FIG. 1).

The enzymatic removal of GalNAc-extended B-antigens and B-antigens from RBCs according to the invention is mediated by two extended B-antigen converting enzymes, where a first enzyme having β-1,3-N-acetylgalactosaminidase activity catalyses the conversion of a GalNAc-extended B antigen to a B-antigen; and a second enzyme having α-1,3-galactosidase activity catalyses the conversion of a B-antigen to an H type 2 antigen (FIG. 1) or other H types, depending on the presence of these type of chains on the A or B (or AB) RBCs.

The enzymatic removal of extended A-antigens and A-antigens from RBCs according to the invention is mediated by three extended A-antigen converting enzymes, where a first enzyme having α-1,3-N-acetylgalactosaminidase activity catalyses the conversion of an A type 3 extended A-antigen to an H type 3 extended A-antigen; a second enzyme having α-1,2-fucosidase activity catalyses the conversion of the H type 3 extended A-antigen to a Gal-A extended A-antigen; a third enzyme having β-1,3-galactosidase activity catalyses the conversion of the Gal-A extended A-antigen to an A-antigen; and then the first enzyme having α-1,3-N-acetylgalactosaminidase activity catalyses the conversion of the A-antigen to an H type 2 antigen or other H types that occur on the A or B (or AB) RBCs. Note that the second enzyme having α-1,2-fucosidase activity can further catalyse the conversion of the H type 2 (or other H types) antigen to form the Bombay phenotype (FIG. 1).

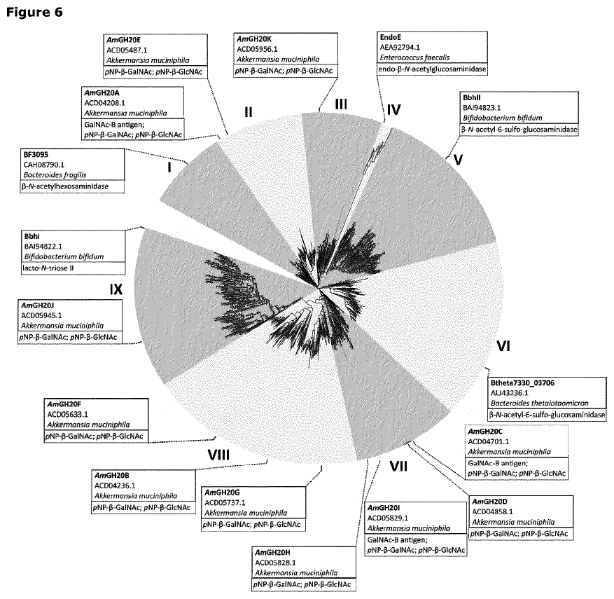

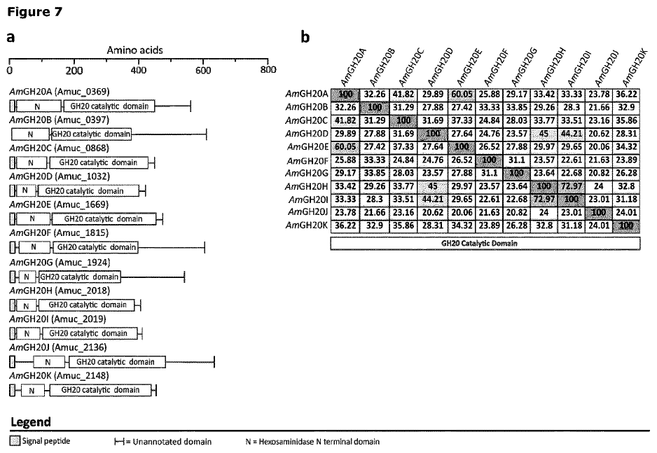

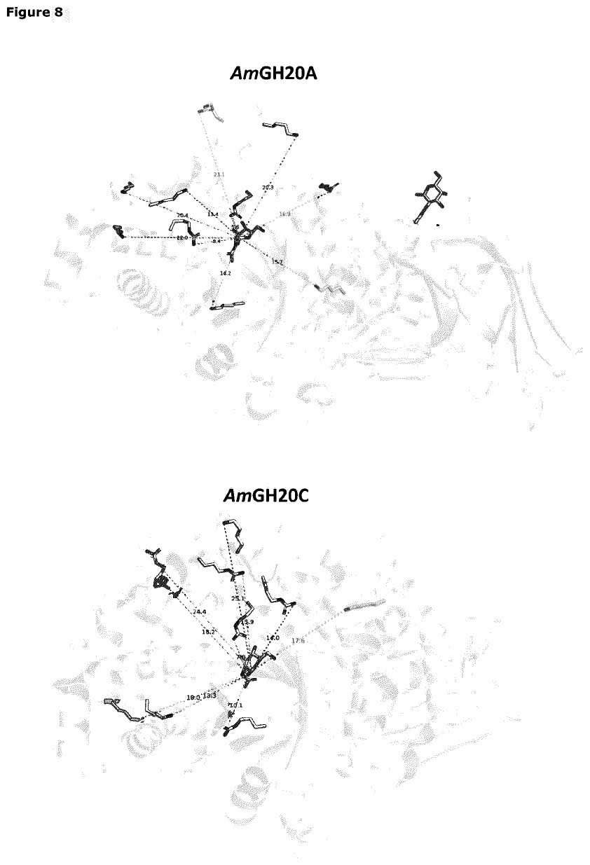

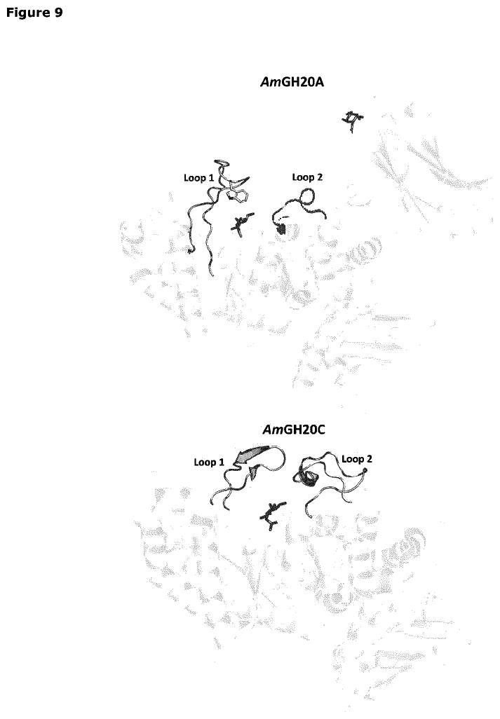

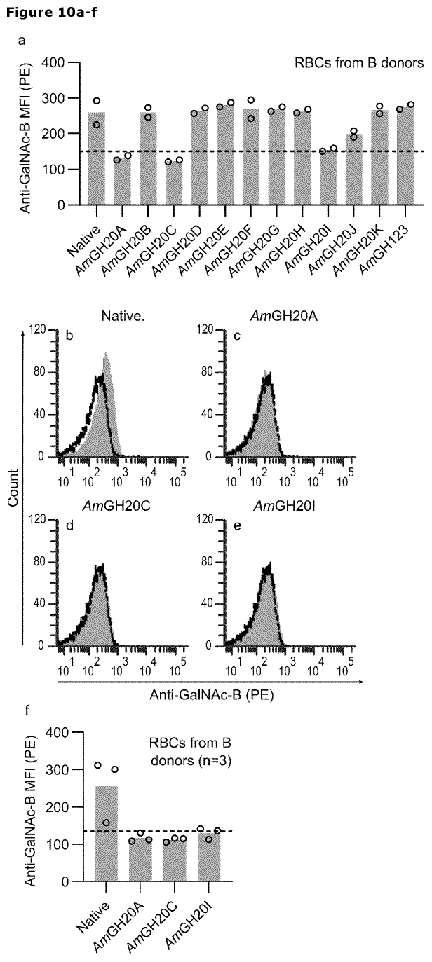

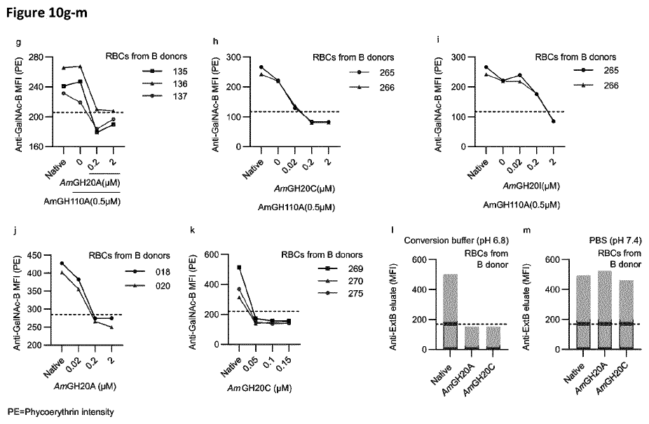

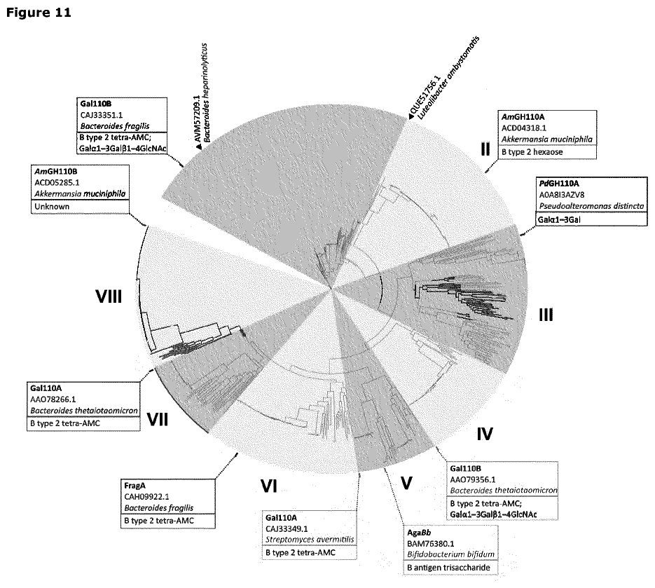

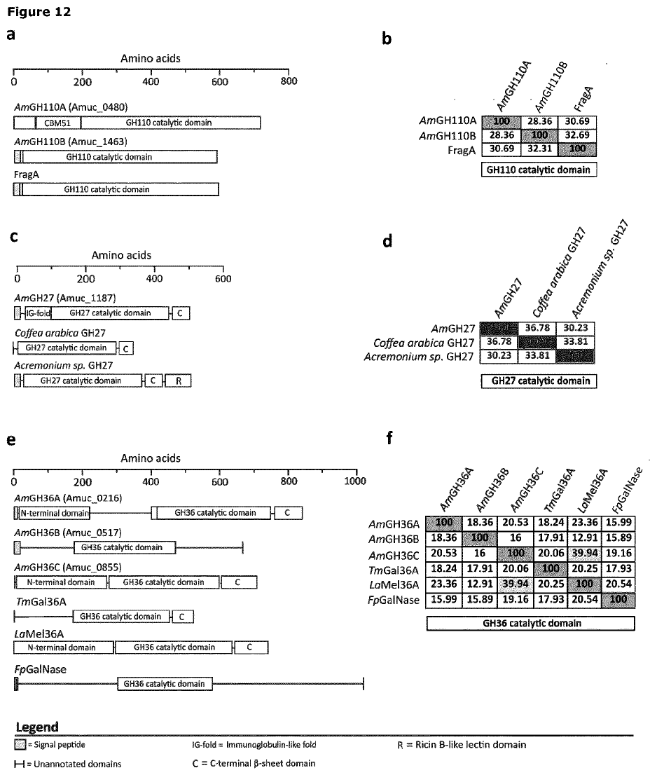

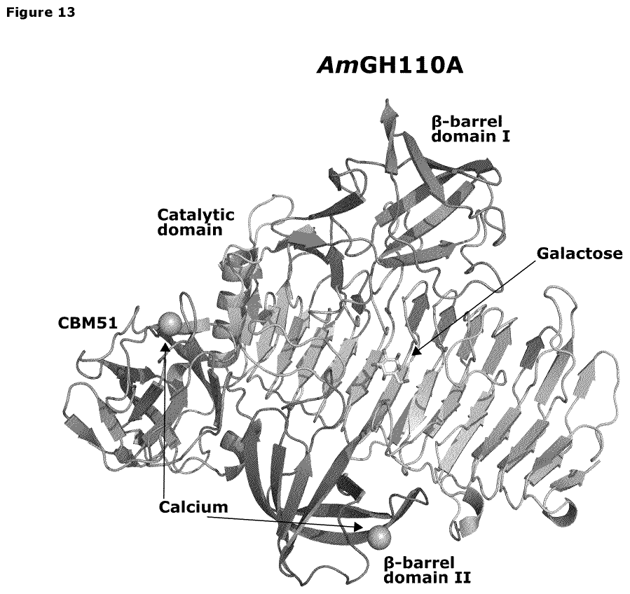

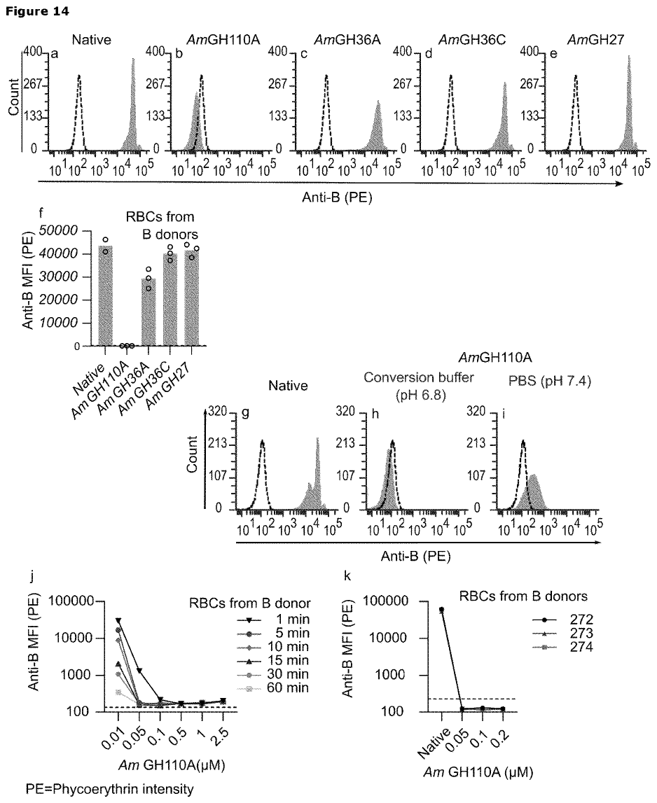

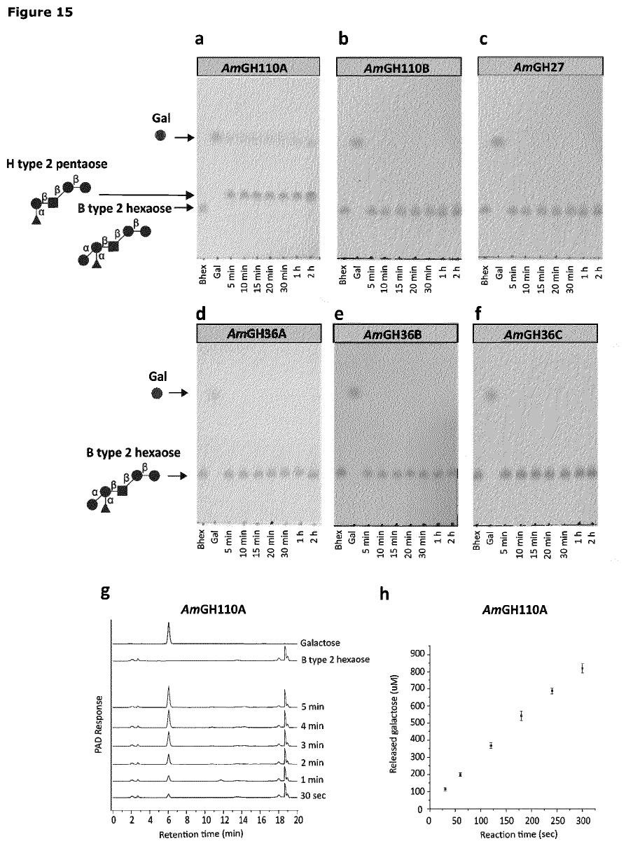

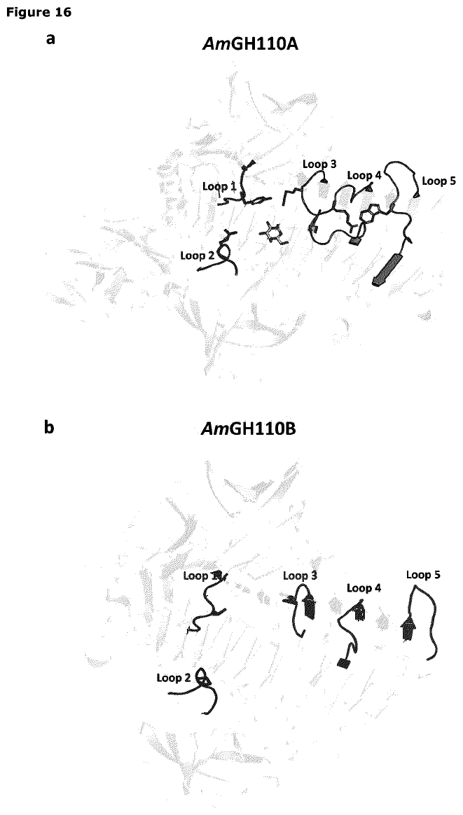

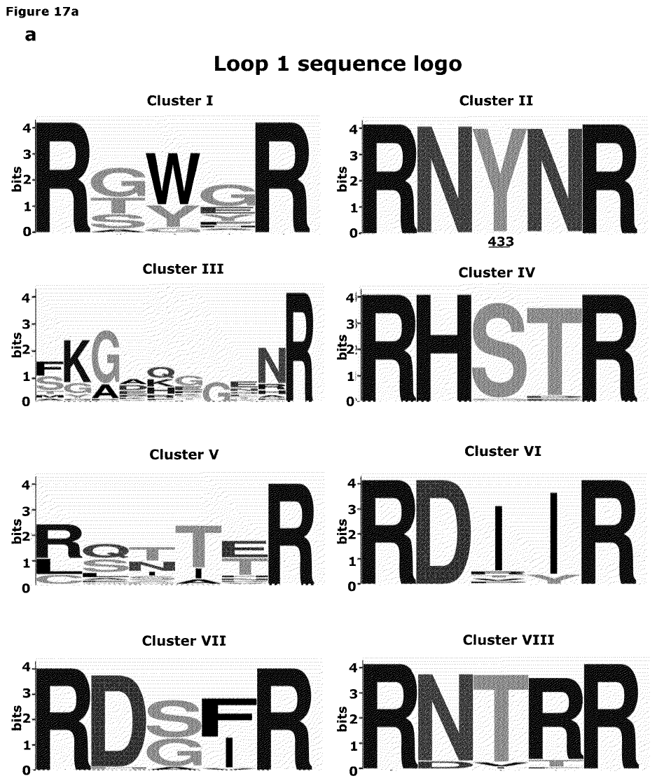

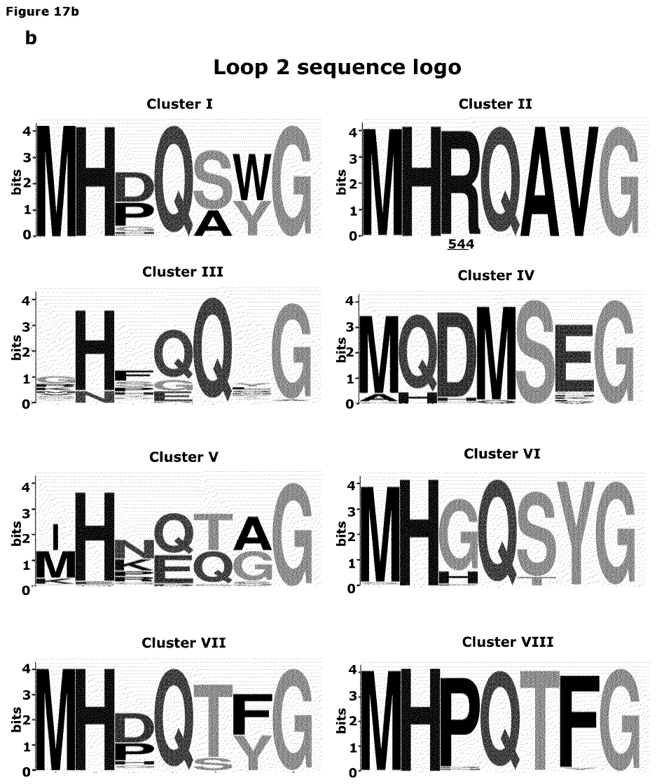

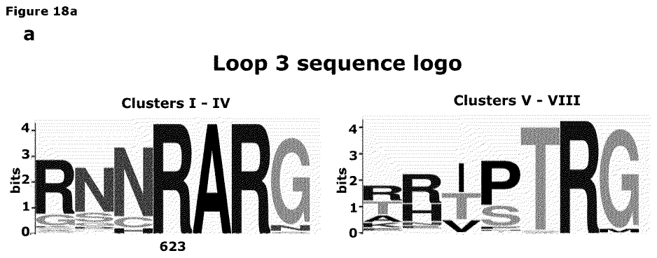

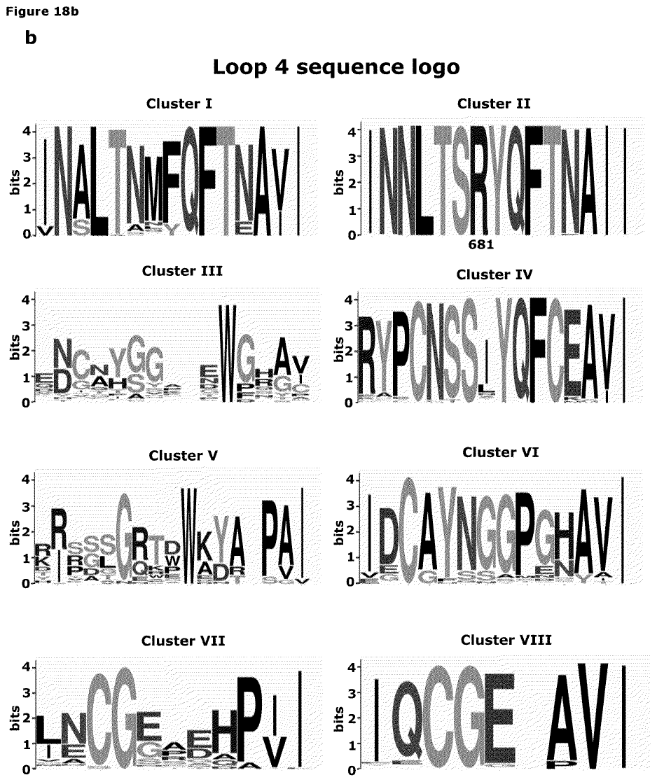

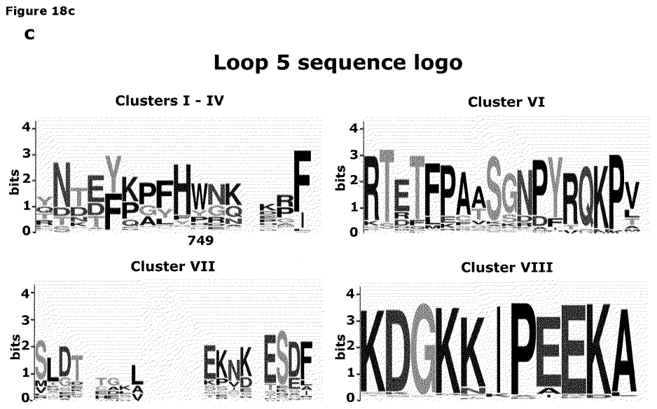

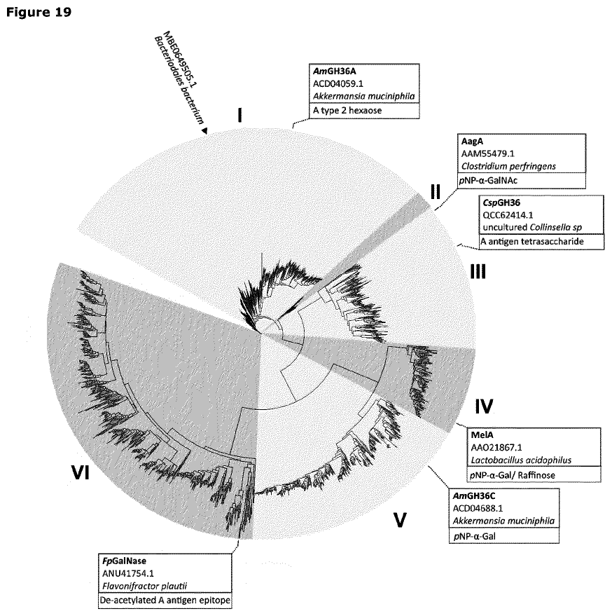

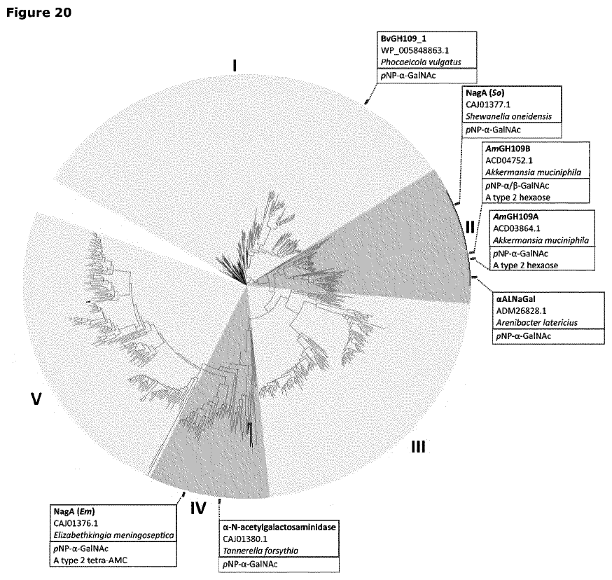

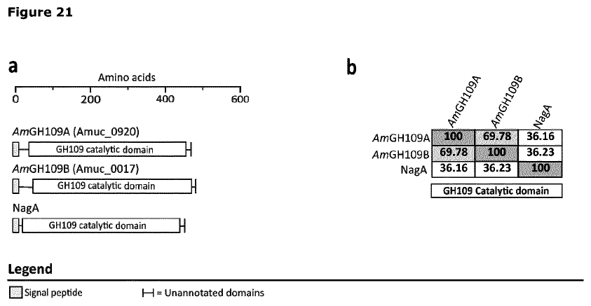

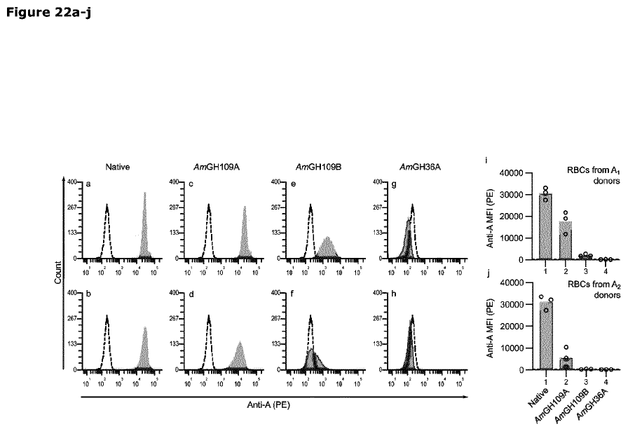

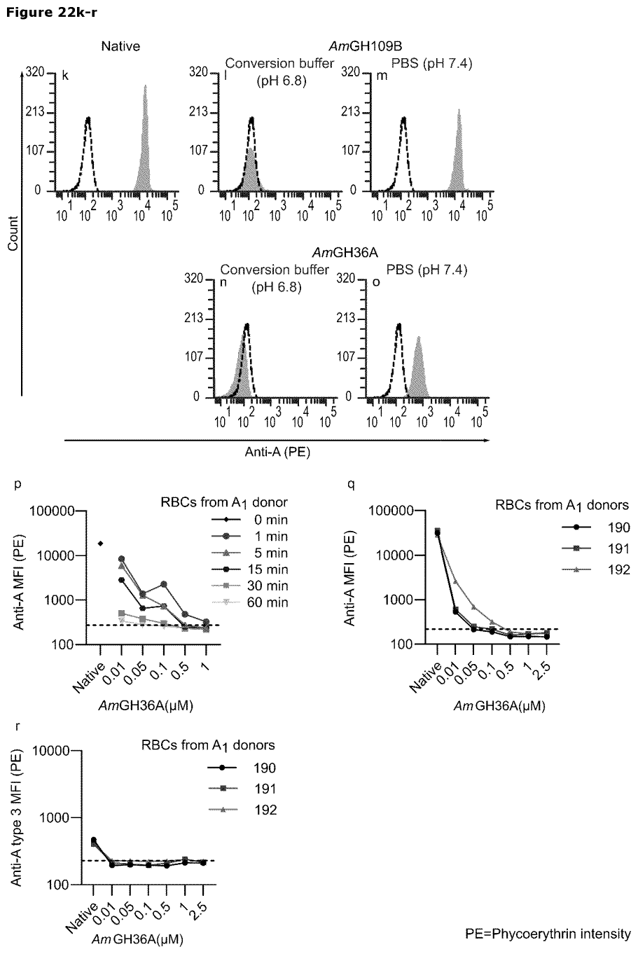

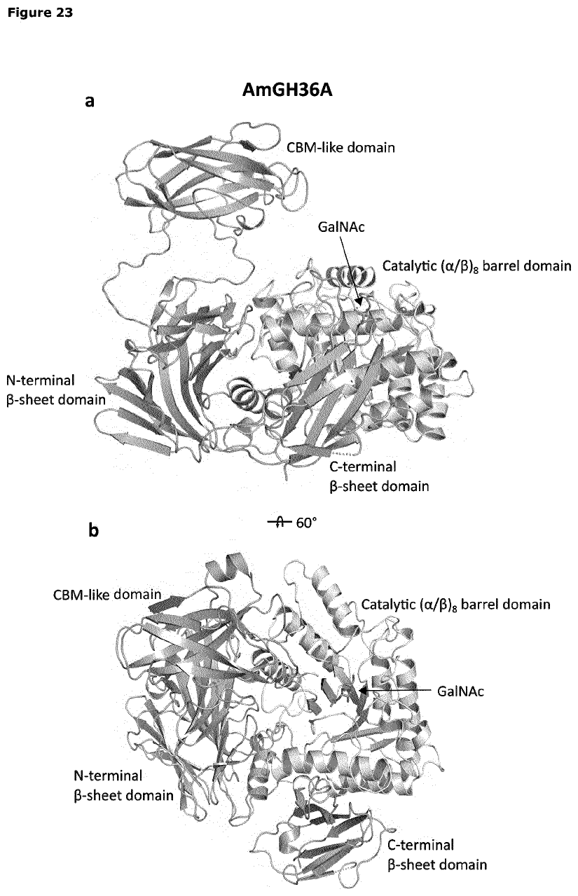

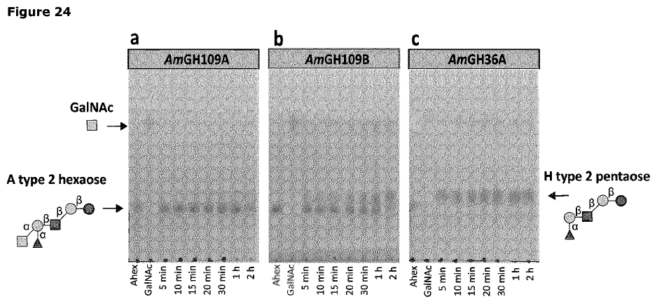

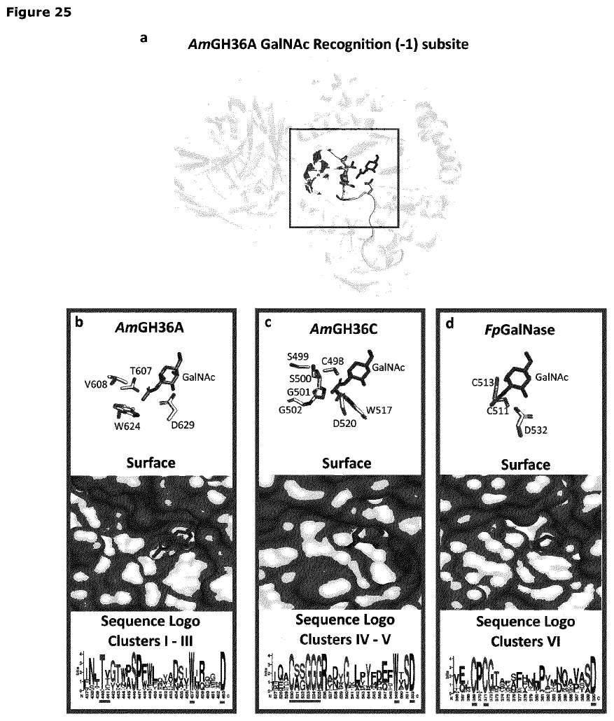

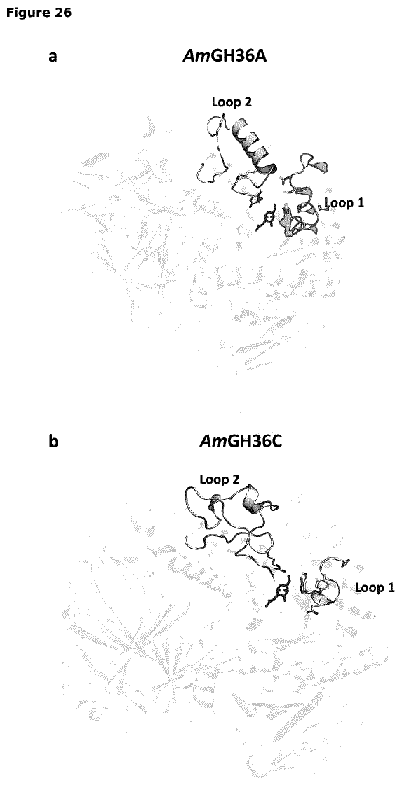

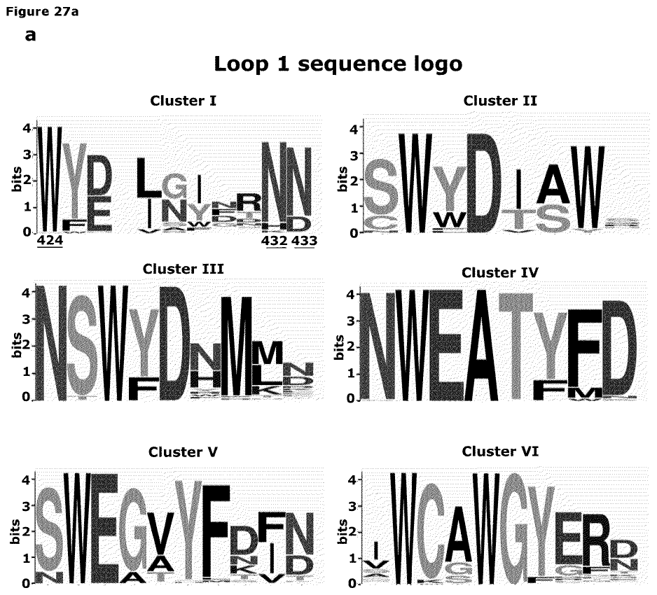

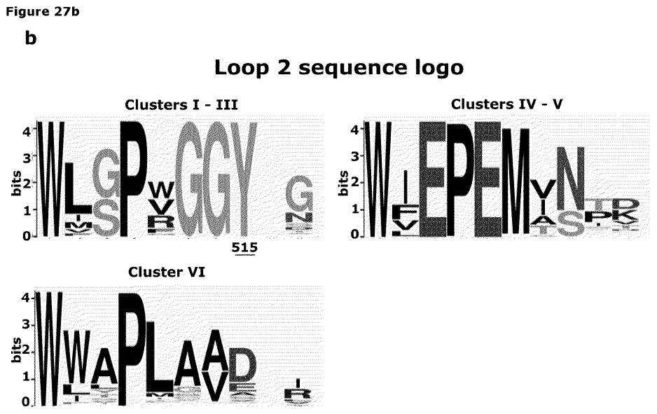

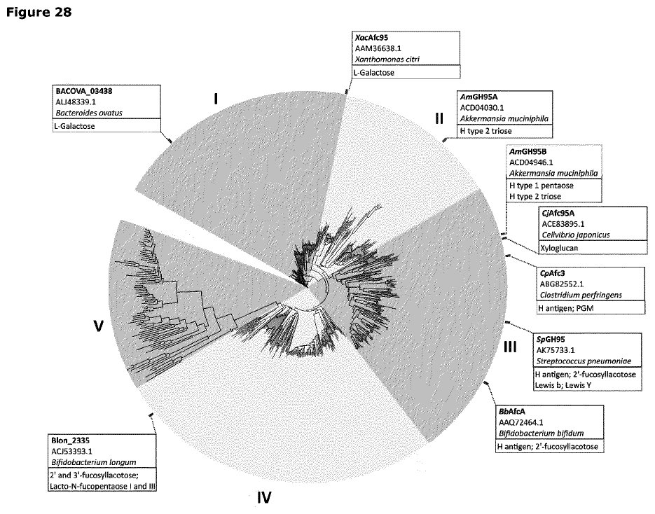

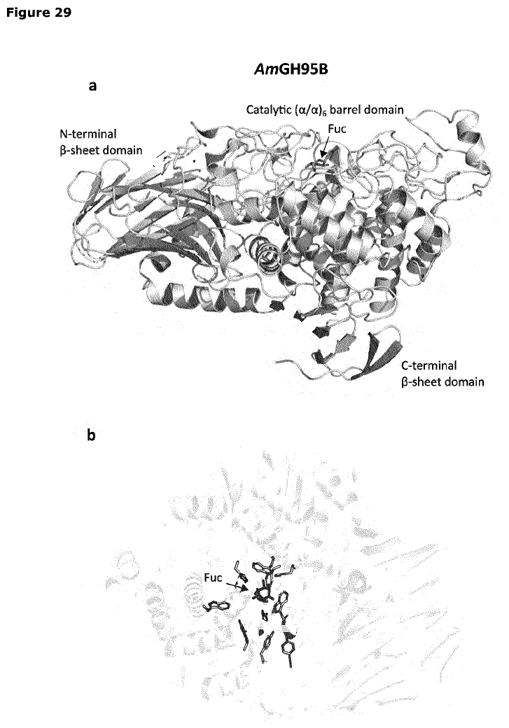

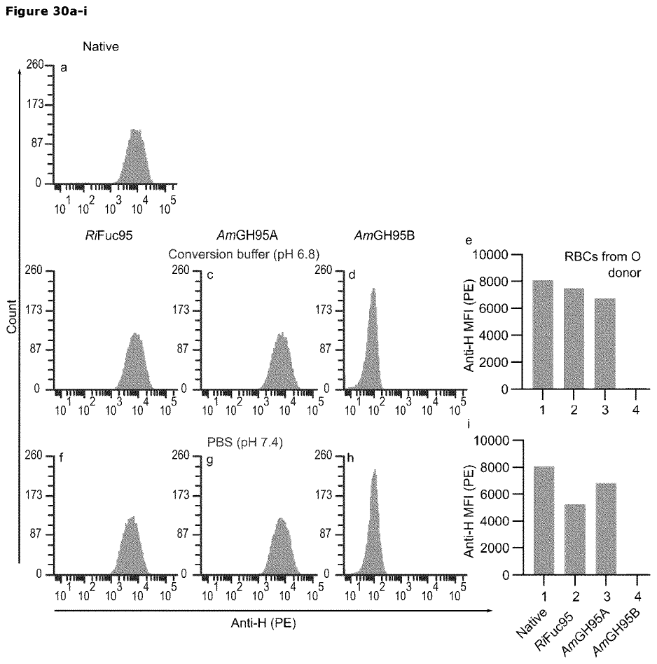

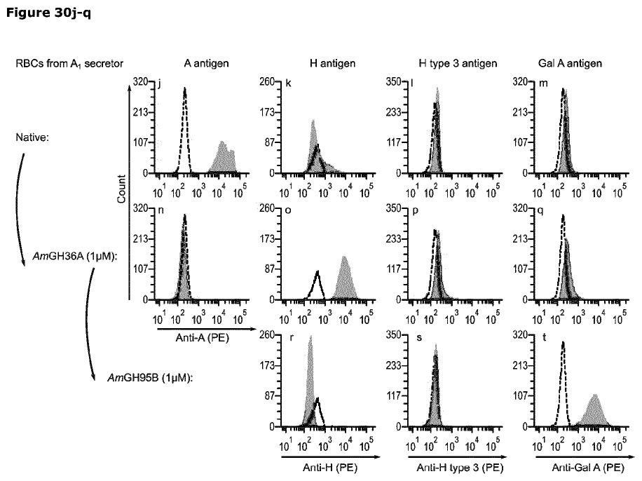

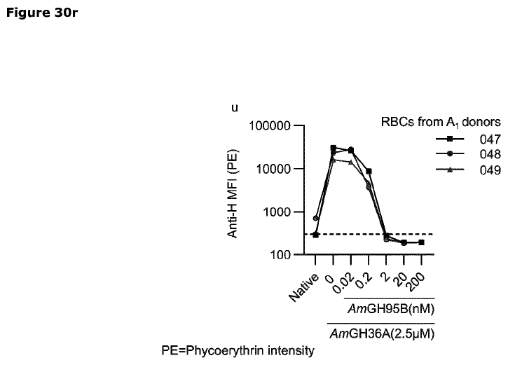

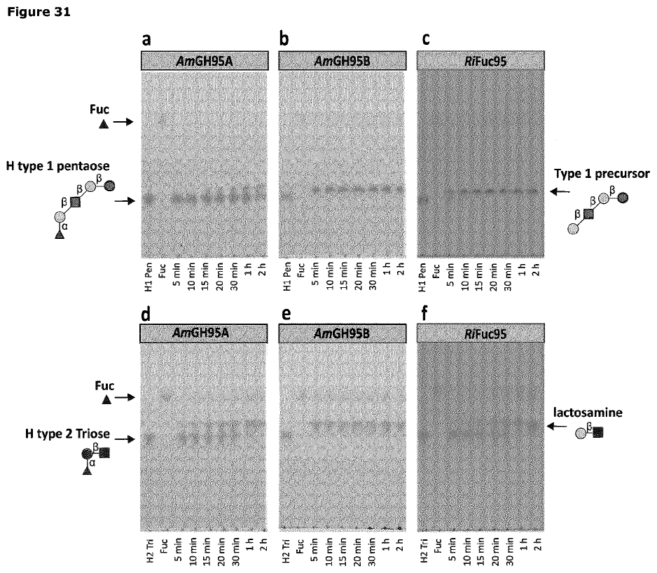

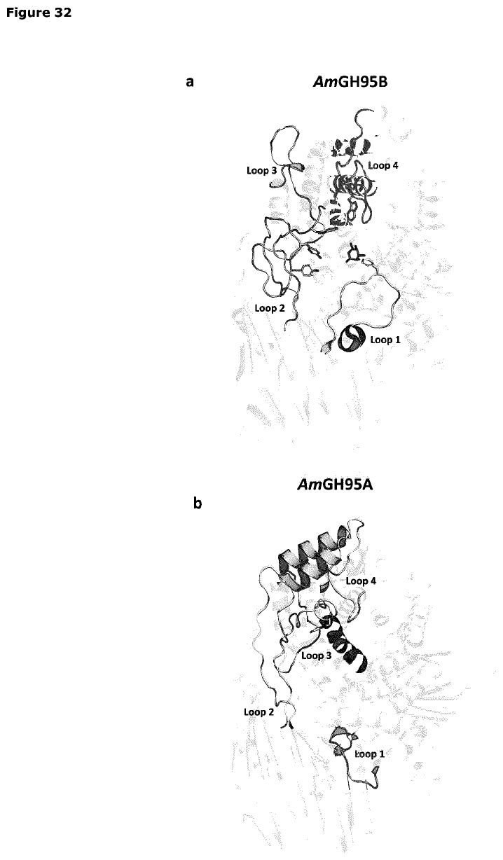

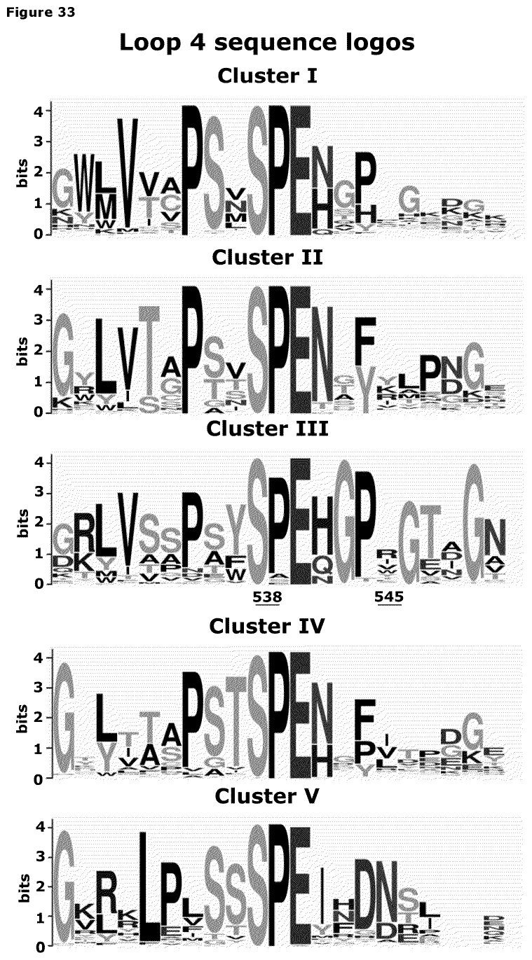

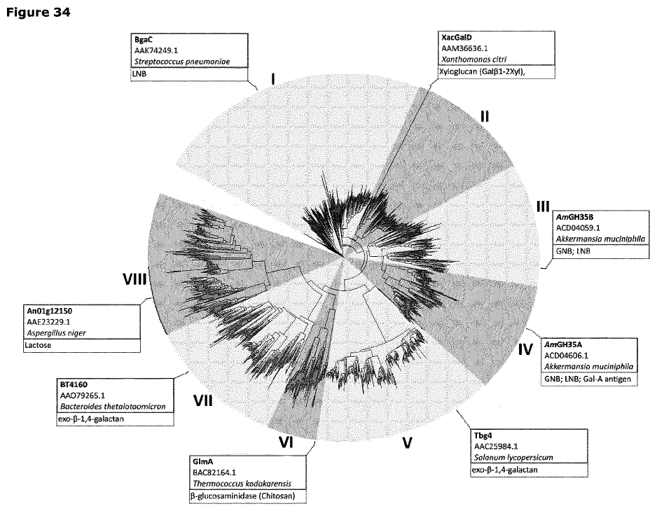

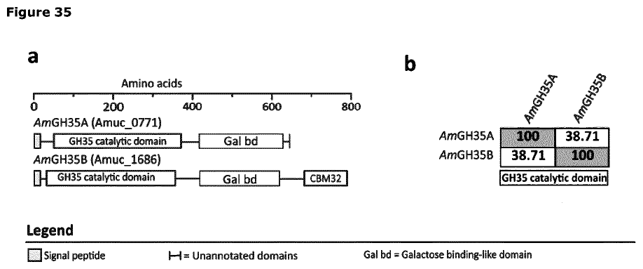

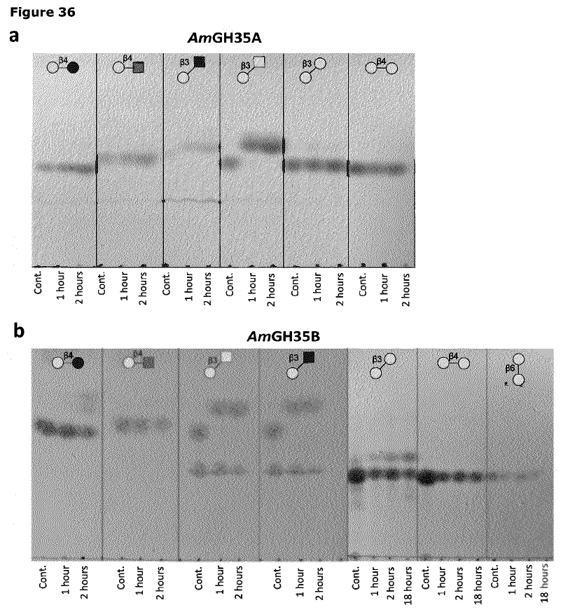

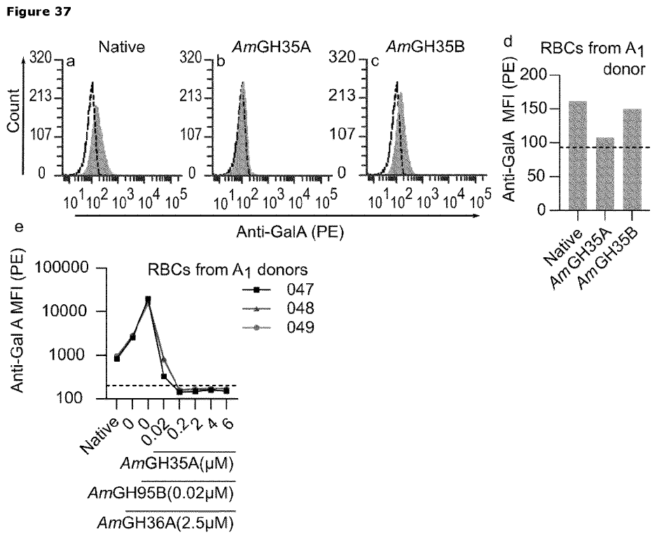

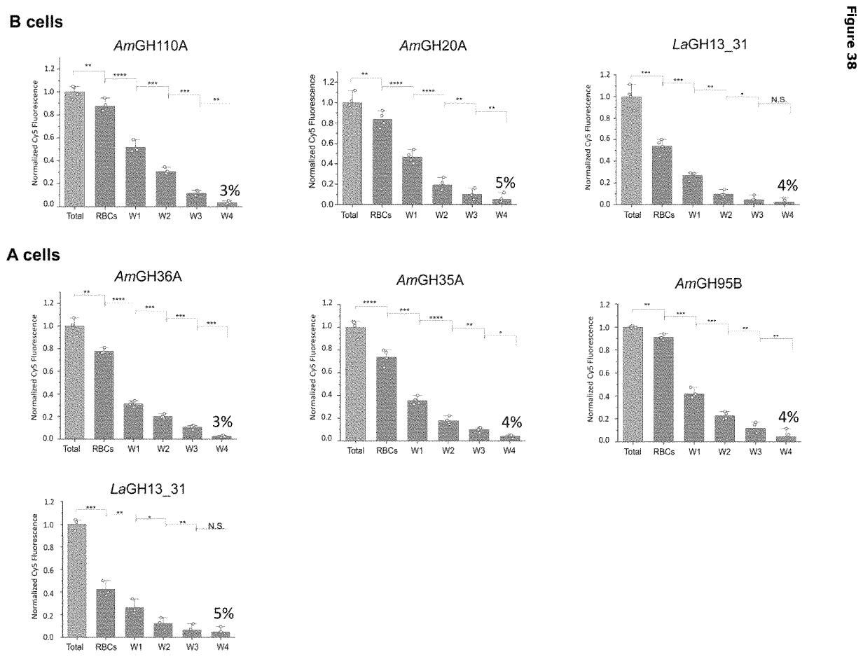

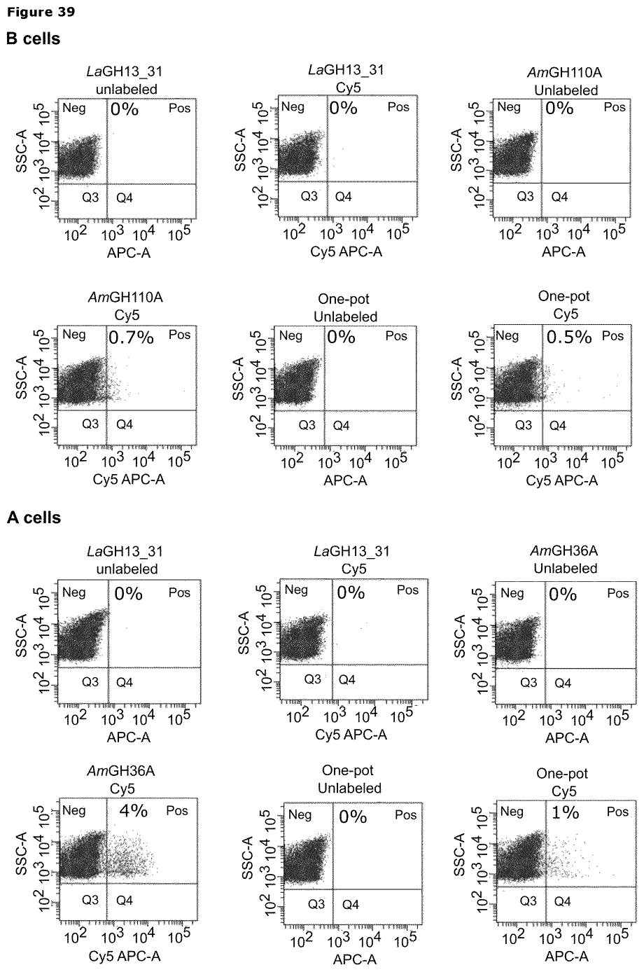

In one embodiment, the extended B-antigen converting first enzyme having β-1,3-N-acetylgalactosaminidase activity belongs to the GH20 enzyme family, preferably an orthologue that populates the same phylogenetic cluster II as AmGH20A, or cluster VII as AmGH20C or AmGH20I (FIG. 6); while the second enzyme having α-1,3-galactosidase activity belongs to the GH110 enzyme family, preferably an orthologue that populates the same phylogenetic cluster II as AmGH110A (FIG. 11) or optionally belongs to the GH36 enzyme family, preferably an orthologue that populates the same phylogenetic cluster V as AmGH36C (FIG. 19).

In one embodiment, the extended A-antigen converting first enzyme having α-1,3-N-acetylgalactosaminidase activity belongs to the GH36 enzyme family, preferably an orthologue that populates the same phylogenetic cluster I as AmGH36A (FIG. 19), or alternatively belongs to the GH109 enzyme family, preferably an orthologue that populates the same phylogenetic cluster II as AmGH109B (FIG. 20). The second extended A-antigen converting enzyme having α-1,2-fucosidase activity and belongs to the GH95 enzyme family, preferably an orthologue that populates the same phylogenetic cluster III as AmGH95B cluster (FIG. 28). The third extended A-antigen converting enzyme having β-1,3-galactosidase activity belongs to the GH35 enzyme family, preferably an orthologue that populates the same phylogenetic cluster IV as AmGH35A (FIG. 34). All phylogenetic clustering is based on multi-sequence alignment based on the catalytic domains of these enzymes (FIGS. 7, 12, 21, and 35)

Enzyme-converted ABO-compatible RBCs obtained by said enzymatic conversion are characterised by lack of the traditional A and B antigens, as well as their known extensions Gal-A, Type 3, A type 3 and ExtB, despite having originated from donors of blood group A, B or AB. They are further characterised by either 1) the expression of H antigen (not including H type 3), which is not a cause of crossmatch reactivity when the RBCs are used for ABO-universal blood transfusion or 2) the lack of all H antigens, i.e. the Bombay phenotype, to which no cross-match-reactive antibodies normally occur.

Accordingly, in a further embodiment, the invention provides a blood product comprising the population of enzyme-converted ABO-compatible RBCs of the invention. The blood product comprises the population of enzyme-converted ABO-compatible RBCs re-constituted as a cell suspension in a medium suitable for transfusion. Such medium may e.g. be isotonic saline, saline-adenine-glucose-mannitol (SAG-M), AS3 or the like (Valeri C R et al., 2008).

The treated RBCs are washed 4 times or a sufficient number of times or variations of this wash to preclude enzyme carry over to the enzyme-converted blood product.

### II: Enzyme Cocktails for RBC Conversion

In a second embodiment, the invention provides a composition or kit for enzyme-conversion of incompatible A or B (or AB) RBCs to enzyme-converted ABO-compatible RBCs, according to the first embodiment (I), comprising purified enzymes characterized as:
β-1,3-N-acetylgalactosaminidase and α-1,3-galactosidase, and/or
α-1,3-N-acetylgalactosaminidase, α-1,2-fucosidase and β-1,3-galactosidase.

In one embodiment, the β-1,3-N-acetylgalactosaminidase enzyme converts the extended B-antigen to a B-antigen, and belongs to the GH20 enzyme family, and is preferably a member of the same phylogenetic cluster as AmGH20A or AmGH20C and AmGH20I; while the α-1,3-galactosidase converts the B-antigen to an H type 2 antigen and belongs to the GH110 enzyme family, and is preferably a member of the same phylogenetic cluster as AmGH110A.

In one embodiment, the α-1,3-N-acetylgalactosaminidase enzyme converts the A type 3 extended A-antigen to an H type 3 extended A-antigen, and belongs to the GH36 enzyme family, and is preferably a member of the AmGH36A phylogenetic cluster; the α-1,2-fucosidase catalyses the conversion of the H type 3 extended A-antigen to a Gal-A extended A-antigen and preferably belongs to the AmGH95B cluster; while the β-1,3-galactosidase catalyses the conversion of the Gal-A extended A-antigen to an H type 2 antigen and preferably belongs to the AmGH35A cluster. The α-1,2-fucosidase can further catalyse the conversion of the H type 2 antigen to the Bombay phenotype (FIG. 1).

The source of enzymes for the enzyme-conversion of A or B (or AB) RBCs to enzyme-converted ABO-compatible RBCs (enzyme cocktails) includes the atypical dedicated mucolytic gut symbiont, Akkermansia muciniphila.

The composition or kit comprising the purified enzymes may provide said purified enzymes solubilized in an aqueous solution (e.g. conversion buffer) or in dried form (e.g. powder). The kit may provide the individual enzymes of said purified enzymes in individual containers in either dried or solubilized form.

### II.i β-1,3-N-Acetylgalactosaminidase

The β-1,3-N-acetylgalactosaminidase is a member of the GH20 family, preferably an orthologue that populates the same phylogenetic cluster II as AmGH20A, or cluster VII as AmGH20C or AmGH20I in the GH20 phylogenetic tree (FIG. 6, FIG. 7). Members of these GH20 phylogenetic clusters comprise two GH20 domains, an N-terminal hexosaminidase domain (amino acids 18-163 in SEQ ID NO.: 2) and the catalytic (β/α)₈ TIM barrel domain, (amino acids 164-552 in SEQ ID NO.: 2), harboring the active site of the enzyme (AmGH20A numbering for both domains). In addition, certain GH20 enzymes may have additional C-terminal domains, for example, at least a C-terminal CBM-like domain (amino acids 553-665 in SEQ ID NO.: 2; AmGH20A numbering) attached to the catalytic domain by a short linker (amino acids 535-552 in SEQ ID NO.: 2, AmGH20A numbering). Such domains may display binding activities for glycans, e.g., with a terminal N-acetylgalactosamine. The presence of the CBM, however, is not essential for the activity on RBCs, based on the activity of AmGH20C and AmGH20I, and other GH20 enzymes lacking the C-terminal CBM-like domain (FIG. 7). The active site surfaces of AmGH20A and AmGH20C have 10 and 9 positively charged residues within 25 & from the anomeric C1 carbon of the bound GalNAc or GlcNAc, respectively (FIG. 8), which is reflected by the high pI value of these enzymes (Table 1). The active site architecture is key for the recognition of the extended GalNAc-B epitope, since only 3 out of 11 enzymes were active on this epitope. Thus, the positive charges that flank the active site, appear to be a necessary, but not sufficient condition for high conversion activity of antigens on RBCs. The potential rationale for this is that surfaces of RBC are heavily negatively charged due to the abundance of sialylated glycans. The active site pocket of AmGH20A and AmGH20C is shallow, solvent exposed, and mainly surrounded by a flat surface topology apart from a single long loop (Loop 1) in AmGH20A (amino acids 223-246 in SEQ ID NO.: 2, AmGH20A numbering) flanking the side of the active site pocket with an additional aromatic platform, Trp233, and a second loop (loop 2) in AmGH20C (FIG. 9). This is most likely an evolutionary adaptation of the enzymes allowing them easier access to mucin-derived glycans, and by extension, the glycan surfaces of RBCs (FIGS. 8, 9).

A β-1,3-N-acetylgalactosaminidase according to the invention is characterized by having a catalytic (β/α)₈ TIM barrel domain, wherein the amino acid sequence of said domain has 48% preferably at least 61% sequence identity to either amino acid residues 164-552 of SEQ ID NO.: 2 comprising the catalytic domain of AmGH20A, or at least 34% sequence identity to amino acid residues 142-532 of SEQ ID NO. 4 comprising the catalytic domain of AmGH20C. Additionally the β-1,3-N-acetylgalactosaminidase is characterized by an isoelectric point, pI value >7.5, which results in an active site flanked with positive electrostatic surface potential. The β-1,3-N-acetylgalactosaminidase may, in some embodiments, harbor a CBM-like domain that displays carbohydrate binding activity.

The β-1,3-N-acetylgalactosaminidase is preferably able to convert GalNAc-B antigens on B type RBCs in a B type blood treatment at 38% hematocrit with β-1,3-N-acetylgalactosaminidase at a concentration of <2 μM within 30 min. (FIG. 10).

TABLE 1
Predicted properties for enzyme candidates for
conversion of B and extended GalNAc B antigens.

| Target | | Signal | Mw | ε₂₈₀ | |
|---|---|---|---|---|---|
| antigen | Enzyme | peptide^{a} | (kDa) | M⁻¹ cm⁻¹ | pI |
| B | AmGH110A | 57(63)^{b} | 81.6 | 1.00 | 8.87 |
| | AmGH110B | 21 | 63.8 | 0.95 | 8.64 |
| | AmGH27 | 21 | 55.8 | 2.02 | 8.58 |
| | AmGH36A | 24(33)^{b} | 91 | 1.84 | 8.95 |
| | AmGH36B | 23/24 | 72 | 1.71 | 8.89 |
| | AmGH36C | No SP | 80.8 | 1.80 | 6.09 |
| | AmGH20A | 19 | 72.8 | 1.38 | 8.92 |
| GalNAc-B | AmGH20B | No SP | 82.3 | 2.04 | 9.2 |
| | AmGH20C | 28 | 61.8 | 1.50 | 7.82 |
| | AmGH20D | 17 | 59 | 1.51 | 8.9 |
| | AmGH20E | 24 | 61.4 | 1.51 | 9.08 |
| | AmGH20F | 23 | 83.3 | 1.73 | 8.79 |
| | AmGH20G | 23 | 75.1 | 1.87 | 8.95 |
| | AmGH20H | 21 | 56 | 1.77 | 8.56 |
| | AmGH20I | 31 | 57.6 | 1.72 | 9 |
| | AmGH20J | 19 | 83.7 | 1.30 | 9.04 |
| | AmGH20K | 25 | 61.4 | 1.68 | 5.89 |

^{a}The signal peptide (number of amino acids) as predicted by SignalP (V.6.0) and deepTMHMM (V.1.0.20).
^{b}The starting amino acid number in the recombinant enzyme is indicated in parenthesis. The absence of parenthesis means that the construct starts at the first amino acid following the signal peptide. The computed properties in the table are based on the amino acid sequence of recombinant enzymes that comprise the mature peptides lacking signal peptides fused to a hexa-histidine C-terminal tags. The properties were computed using Expasy ProtParam, with the extinction coefficients assuming using all Cys residues are reduced.

### II.ii α-1,3-Galactosidase

The α-1,3-galactosidase is a member of the GH110 enzyme family, preferably an orthologue that populates the same phylogenetic cluster II as AmGH110A (FIG. 11). AmGH110A comprises an N-terminal CBM51 (amino acids 64-212 in SEQ ID NO.: 8) domain containing a putative Ca²⁺-binding site. Hereafter, the catalytic module consists of a parallel β-helix structure containing 12 turns (amino acids 213-795), flanked by two additional small β-barrels (β-barrel I & β-barrel 2) domains on either side. One of the β-barrel domains (β-barrel 1 (amino acids 333-433 in SEQ ID NO.: 8) emerges upward adjacent to the active site and almost orthogonal to the plane of the catalytic β-helix domain, while the other β-barrel domain (β-barrel II, amino acids 524-609 in SEQ ID NO.: 8) packs on the side of the catalytic domain at the opposite side of β-barrel 1, and contains a Ca²⁺-binding site (FIG. 11, 12).

The β-barrel I domain (FIG. 13) has significantly reduced loops compared to the non-converting PdGH110B, resulting in a more exposed active site surface, which may facilitate access to glycan substrates within a densely glycosylated region. In addition, the surface surrounding the active site pocket exhibits a positive electrostatic surface potential, whereas PdGH110B has the opposite negatively charged surface charge flanking the active site, despite showing similarly high theoretically predicted isoelectric point (pI=9.00). Therefore, the high pI appears to be a necessary but not sufficient condition for the interaction with RBCs.

As opposed to RBC active AmGH110A (FIG. 14), AmGH110B displays no activity against the B type 2 antigen hexaose (FIG. 15). The amino acid sequence identity between the catalytic domain of AmGH110A (located at amino acid residues 213-795 of SEQ ID NO.: 8) and AmGH110B is 28%. A structural comparison between the two enzymes revealed key differences in loops 1-5 on the active side face of the enzyme (FIG. 16). A phylogenetic analysis also reveals that the enzymes segregate in different clusters of the phylogenetic tree of GH110 (FIG. 11), with AmGH110A belonging to cluster II. The amino acid sequence identity between the catalytic domain (amino acid residues 213-795 of SEQ ID NO.: 8) of AmGH110A and the catalytic domain of the furthest member of the same cluster, namely Luteolibacter ambystomatis (QUE51756.1) is 49.28%. L. ambystomatis is a member of the Verrucomicrobiales order that harbors A. muciniphila. All the sequences of this cluster are taxonomically related, stemming from the Verrucomicrobia phylum. By contrast, AmGH110B (SEQ ID NO.: 34) populates the distant cluster VIII. A key signature of AmGH110A is the presence of a Tyrosine (Y433), on loop 1 (residue 431-440 in SEQ ID NO.: 8, AmGH110A numbering) (FIG. 17) connecting the C-terminal end of the B1 sheet domain to the catalytic β-helix domain (FIG. 16a). This tyrosine is invariant in cluster II, while it is chemically conserved either as W or Y in the corresponding position (W: 65%; Y: 28.2%) in the adjacent cluster I (FIG. 17a). The amino acid sequence identity between the catalytic domain (amino acid residues 213-795 of SEQ ID NO.: 8) of AmGH110A and the catalytic domain of the furthest member of Cluster I, namely (AVM57209.1 from Bacteroides heparinolyticus, pI value 6.73) is 36.48%. All other clusters of the GH110 phylogenetic tree lack this conserved aromatic residue in the loop 1 motif (FIG. 17a). In AmGH110A, Y433 is in close proximity to an arginine (R544) (about 7 A) on loop 2, consisting of residues 540-554 of SEQ ID NO.: 8 (AmGH110A numbering). Interestingly, R544 is an invariant and unique residue in cluster II (FIG. 17b), which contributes to the positive potential around the active site, and it may also form polar interactions with a substrate saccharide unit stacked onto Y433. The corresponding position to R544 in AmGH110B is a proline, which is 95% conserved in cluster VIII. Another distinguishing feature is the presence of an invariant arginine (R623) in the middle of loop 3 (residues 620-627 of SEQ ID NO.: 8, AmGH110A numbering), which is conserved in clusters I-IV (FIG. 18a). Structural alignment to PdGH110B in complex with α-galactobiose (βDB: 7JWF) reveals that the corresponding arginine is involved in the binding of the β-Gal at the +1 subsite. For clusters V-VIII, the corresponding conserved amino acid in this loop is either a proline (61.1%) or a serine (28.6%), with only 2.4% of sequences containing an arginine. Another difference between AmGH110A and AmGH110B, is the elongation of loops 4 (amino acids 676-690 of SEQ ID NO.:, 8, AmGH110A numbering) and 5 (amino acids 742-754 of SEQ ID NO.: 8, AmGH110A numbering) in AmGH110A. Loop 4, harbors another invariant arginine (R681) from cluster II (FIG. 18b), which further contributes to the positive surface potential of the active site side of the enzyme, and is in close proximity to another conserved aromatic residue on loop 5 (W749, 54.2% conserved in clusters I-IV) (FIG. 18c). Clusters VII and VIII that harbors AmGH110B, both have shortened loops lacking the arginine and the aromatic residue. While the aromatic residue of the elongated loop 5 is present in several of the clusters, the corresponding arginine (R681) is only observed in about 31% of the enzymes from cluster V, all from the pathogenic bacterium Erysipelothrix rhusiopathiae. Strikingly, amongst all biochemically characterized GH110 enzymes active on the B antigen, only AmGH110A has a high pI, whereas the others had pI values lower than 7 (Table 2), which is deemed to be an important signature for good affinity to RBC surfaces.

TABLE 2
Isoelectric point (pI) of characterized GH110
enzymes that are active against the B antigen.

| Enzyme | Source | Genbank accession | pI |
|---|---|---|---|
| AmGH110A | Akkermansia mucinphila | ACD04318.1 | 8.87 |
| FragA | Bacteroides fragilis | CAH09922.1 | 6.99 |
| Gal110B | Bacteroides fragilis | CAJ33351.1 | 6.53 |
| Gal110A | Bacteroides thetaiotaomicron | AAO78266.1 | 6.36 |
| Gal110B | Bacteroides thetaiotaomicron | AAO79356.1 | 5.76 |
| AgaBb | Bifidobacterium bifidum | BAM76380.1 | 5.26 |
| Gal110A | Streptomyces avermitilis | CAJ33349.1 | 6.17 |

In a preferred embodiment, the α-1,3-galactosidase is characterized by having a catalytic domain, wherein the amino acid sequence of said domain has ≥36%, sequence identity to amino acid residues 213-795 of SEQ ID NO. 8 comprising the catalytic domain of AmGH110A.

In a preferred embodiment, the α-1,3-galactosidase is additionally characterised by a catalytic domain having a pI value of higher than 7.5.

Furthermore, the α-1,3-galactosidase preferably has activity against the B-type 2 hexaose (Table 3).

TABLE 3
Normalized rates of A. muciniphila enzyme candidates active
on A type and B type antigens and extended forms thereof.

| | | Normalized Rate | Specific Activity |
|---|---|---|---|
| Enzyme | Substrate | (V₀/E) (s⁻¹) | (U/mg) |
| AmGH110A | B type 2 hexaose | 121.9 ± 3.2 | 89.6 ± 2.3 |
| (α-D-1,3-Galactosidase) | | | |
| AmGH95A | H type 1 pentaose | 101.1 ± 3.5 | |
| (α-L-1,2-Fucosidase) | | | |
| AmGH36A | A type 2 hexaose | 127.3 ± 4.1 | |
| (α-D-1,3-GalNAcase) | | | |
| AmGH109B | A type 2 hexaose | 0.57 ± 0.09 | |
| (α-D-1,3-GalNAcase) | | | |

In a preferred embodiment, the α-1,3-galactosidase is one that is able to remove the terminal non-reducing α-galactosyl in the B type 2 hexaose with a normalised rate >15 s⁻¹

In a more preferred embodiment, the α-1,3-galactosidase is one that is able to remove the terminal non-reducing α-galactosyl in the B type donor cells (at 38% hematocrit) within 30 min at room temperature (20-24° C. at a concentration <5 μM. (FIG. 14).

### II.iii β-1,3-N-Acetylgalactosaminidase and α-1,3-Galactosidase

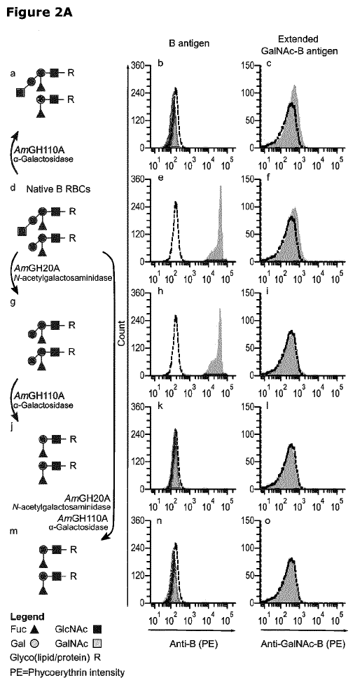

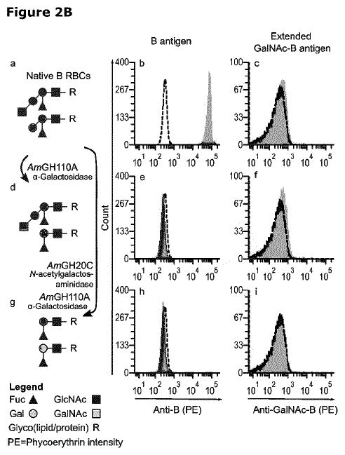

A composition comprising the enzymes according to II.i and II.ii having β-1,3-N-acetylgalactosaminidase and α-1,3-galactosidase activity can be used to remove extended B antigens and B-antigens from B RBCs, as illustrated in Example 2.v and FIG. 2. Furthermore, a composition comprising the enzyme according to II.ii can be used to remove B-antigens from B RBCs. This combination should be able to reduce (or abolish) the positive crossmatches with recipient plasma as compared to treatment with only a B type converting enzymes (FIG. 5)

### II.iv α-1,3-N-Acetylgalactosaminidase

The α-1,3-N-acetylgalactosaminidase is a member of the GH36 enzyme family, preferably an orthologue that populates the same phylogenetic cluster I as AmGH36A (FIG. 19), or alternatively belongs to the GH109 enzyme family, preferably an orthologue that populates the same phylogenetic cluster II as AmGH109B (FIG. 20). Both AmGH36A and AmGH109B are able to convert the A antigen on RBCs (FIG. 22), although AmGH36A is far more efficient. The AlphaFold model generated for AmGH36A reveals a unique organisation among previously described GH36 structures (FIG. 23), consisting of four domains, including an N-terminal β-sheet domain (amino acids 32-219 and 366-384 in SEQ ID NO.: 10) with an attached unannotated CBM-like domain (amino acids 220-365 in SEQ ID NO.:10), linked to the catalytic (β/α)₈ barrel domain (amino acids 385-749 in SEQ ID NO.:10) and a C-terminal β-sheet domain. The unique CBM-like domain is positioned above the active site, which resembles a Cobra attack pose, previously observed in other mucin-specific modular enzymes, notably from A. muciniphila.

The amino acid sequence of the catalytic domain of AmGH36A (amino acids 385-749 of SEQ ID NO.: 10) shares 30.18% and 27.70% sequence identity with the catalytic domains of the two previously characterized α-N-acetylgalactosaminidases of GH36 enzyme family from Clostridium perfringens and an uncultured Collinsella sp., respectively. AmGH36A displays high activity against the A type 2 hexaose (FIG. 24) and high efficiency in cleavage of the A antigen and the extended A type 3 antigen on the surfaces of RBCs (FIG. 22). The amino acid sequence of said catalytic domain of AmGH36A shares 20.53% amino acid sequence identity with the catalytic domain of the nonconverting putative α-galactosidase AmGH36C, and 15.99% amino acid sequence identity to the catalytic domain of α-galactosaminidase FpGalNAse. FpGalNAse specifically hydrolyses the terminal non-reducing galactosamine that is generated from the de-acetylation of the A antigen's terminal nonreducing GalNAc.

The phylogenetic analysis of GH36 enzyme family sequences reveal the segregation of the enzymes with different specificities into different clusters (FIG. 19).

The amino acid sequence of amino acids 385-749 of SEQ ID NO.: 10, comprising the catalytic domain of AmGH36A from cluster I of the GH36 enzyme family, shares 36.57% sequence identity with the catalytic domain of the furthest removed member in the cluster (MBE0649505.1 from Bacteriodales bacterium, pI value: 8.62). AmGH36C populates cluster IV of the GH36 enzyme family. Using sequence alignment and AlphaFold generated models, identified a few structural signatures that correlate to the catalytic efficiency of AmGH36A against A type antigens. Major differences in the organization of a loop adjacent to subsite −1 around the C2 atom of GalNAc explains the accommodation of the N-acetyl group of GalNAc (FIG. 25) in an apolar pocket consisting of a highly conserved threonine (T607) (95.3%), a following valine (V608)/threonine (39.8%/30.1%), and a tryptophan (W624) (99.2%), all AmGH36A numbering [SEQ ID NO.: 10], in enzymes of GH36 clusters I-III (including AmGH36A). In GH36 clusters IV and V harboring α-galactosidases (including AmGH36C), a conserved glycine motif blocks the N-acetyl specificity pocket and interacts with the 02 of galactose. The loop adopts a different conformation with the glycine amide backbone and the tryptophan occupying a space about 3 Å away from the nitrogen atom of the GalNAc (corresponding to the placement of the 02 in galactose), thereby blocking the accommodation of the acetyl group of GalNAc. This N-acetyl specific pocket offers a signature to distinguish α-N-acetylgalactosaminidases from enzymes that recognise galactosyl, e.g. α-galactosidases (FIG. 25b,c). In GH36 cluster VI harboring galactosaminidases (FpGalNase), a conserved “CPCG” motif is present occupying that same space. In these sequences, the tryptophan residue is lost. The thiol group of the cysteines are 4.8 Å away from each other in the AlphaFold model, and may potentially form an intermolecular disulfide bond in this region to preserve the loop structure blocking the acetyl group from binding, with the conserved aspartic acid potentially interacting with the amine group of galactosamine (FIG. 25c).

An elongation of loop 1 (amino acids 422-442 of SEQ ID NO.: 10, AmGH36A numbering) by the active site pocket, which contains a conserved “NN” motif, corresponding to N432 and N433, which is located above W424 of the same loop (FIG. 26), may be involved in substrate interactions in the +1/+2 subsite. This elongated loop with the “NN” motif is only observed in GH36 Cluster I and II of the phylogenetic tree, and is lacking in all previously characterized GH36 enzymes (FIG. 27a). Furthermore, loop 2 (residues 509-542 of SEQ ID NO.: 10, AmGH36A numbering) (FIG. 26) in GH36 clades I-III, which harbor the GalNAcases, contains a conserved GGYG motif (FIG. 27b), presenting a flexible region with an aromatic residue. The OH group of the tyrosine (Y515) is 4.8 Å away from the C1 of the GalNAc and could participate in substrate recognition at subsite +1. The shortening of this loop and the insertion of an α-helix contribute to a flat architecture flanking the catalytic site of AmGH36A. Four lysine residues that are presented by this loop contribute to the local positive electrostatic surface potential flanking the active site. Of these, two are conserved within the GH36 cluster I among enzymes from Verrucomicrobia K519 (49.4%) and K534 (26.5%, or corresponding arginine at 17.2%). These features may contribute to enzyme binding to the negatively charged surfaces of RBCs. The α-helix in loop2 is only observed in GH36 clusters I, II, and III (the α-N-acetylgalactosaminidases), however cluster III harbors a more elongated loop as compared to the I and II counterparts.

In one embodiment the α-1,3-N-acetylgalactosaminidase according to the invention is characterized by belonging to phylogenetic cluster I of the GH36 enzyme family, and having a catalytic domain, wherein the amino acid sequence of the domain has >36% sequence identity to amino acid residues 385-749 of SEQ ID NO.: 10 comprising the catalytic domain of AmGH36A. The α-1,3-N-acetylgalactosaminidase may possess a CBM domain, and preferably displays activity against the A type 2 hexaose (Table 3) and A antigens on surface of RBCs.

In one embodiment the α-1,3-N-acetylgalactosaminidase according to the invention belongs to the same phylogenetic cluster II of the GH109 enzyme family as AmGH109B. In a preferred embodiment the α-1,3-N-acetylgalactosaminidase has a catalytic domain, wherein the amino acid sequence of the domain has at least 46% sequence identity to amino acids residues 30-481 of SEQ ID NO. 12 comprising the catalytic domain of AmGH109B.

In a preferred embodiment, the α-1,3-N-acetylgalactosaminidase also has a pI value >7.5.

In a preferred embodiment, the α-1,3-N-acetyl-galactosaminidase is one that can cleave the A type 2 hexaose with a normalised rate >15 s⁻¹ at the standard conditions described herein.

In a more preferred embodiment, the α-1,3-N-acetylgalactosaminidase is one that can cleave the terminal non-reducing α-GalNAc unit from A type blood within 30 min at room temperature in the conversion buffer described here, at a concentration <10 μM. (FIG. 22).

### II.v α-1,2-Fucosidase

The α-1,2-fucosidase is a member of the GH95 enzyme family, preferably an orthologue that populates the same phylogenetic cluster III as AmGH95B (FIG. 28). Members of this cluster, e.g. AmGH95B, comprise a GH95 domain organization of an N-terminal β-sheet domain (amino acids 26-260 of SEQ ID NO.: 14), followed by a long α-helix domain (amino acids 261-357 of SEQ ID NO.: 14), following the catalytic (a/a)₆-barrel domain (amino acids 358-730 of SEQ ID NO.: 14), all with AmGH95B numbering, and a short C-terminal β-domain (FIG. 29a). AmGH95B harbors an exposed active site, with 10 aromatic residues surrounding the fucose in the active site pocket (FIG. 29b). The amino acid sequence of the catalytic domain of AmGH95B (amino acids 358-730 of SEQ ID NO.: 14), shares about 30% sequence identity of the catalytic domain with AmGH95A and 25% sequence identity to the sequence of the catalytic domain of RiFuc95, both of the latter being inactive on H type antigens on the surfaces of RBCs (FIG. 30), despite showing some activity against the H1 pentaose or H2 triose by TLC (FIG. 31). The amino acid sequence of said catalytic domain of AmGH95B shares 41.31% with the sequence of the catalytic domain a GH95 enzyme from Clostridium perfringens (Afc3, Genbank ID: ABG82552.1), which shows activity against the H antigen oligosaccharide and porcine gastric mucin. A phylogenetic analysis of GH95 enzymes retrieved from human gut metagenomes showed that AmGH95A and AmGH95B belong to different clusters (II and III, respectively) (FIG. 28). Interestingly, the closest characterized GH95 to AmGH95B belongs to the soil bacterium Cellvibrio japonicas (CjAfc95A; ACE83895.1), which is shown to release α-fucose from xyloglucan. The amino acid sequence identity between said catalytic domain of AmGH95B and the sequence of the catalytic domain of CjAfc95A is 39.43%. A comparison of the structure of AmGH95B against an AlphaFold generated model of AmGH95A and available structures of GH95 enzymes in PDB, showed some distinct features, which may correlate with the ability of AmGH95B to catalyze reactions efficiently on the surfaces of RBCs. The first loop (loop 1) of the N-terminal domain (residues 46-61 of SEQ ID NO.: 14, AmGH95B numbering) containing a phenylalanine (F53) is elongated, closing over the active site (FIG. 32). Elongation of this loop is unique for only a few sequences in the phylogenetic tree, including AmGH95B and 10 other sequences in cluster III (129 total sequences), including BbafcA from Bifidobacterium bifidum (whose catalytic domain shares 33.47% amino acid sequence identity to said catalytic domain of AmGH95B), as well as 30 sequences from cluster IV (151 total sequences) (FIG. 28). The composition of amino acids in the loop is not conserved and varies between enzymes having the extended loop. In BbafcA, the loop harbors a tryptophan in the corresponding alignment position to the phenylalanine in AmGH95B. A structural comparison reveals that the BbafcA loop contains a small α-helix turn, shortening the reach of the loop from the active site pocket. The phenylalanine (F53) from AmGH95B closing over the active site is not conserved in the GH95 cluster. Another major difference is the relatively shorter loop 2, (residues 93-123 of SEQ ID NO.: 14, AmGH95B numbering) which contains a flexible aromatic platform, GGGYGYG (residues 103-109 of SEQ ID NO.: 14, AmGH95B numbering), likely involved in substrate binding at the +1/+2 subsite. Similarly, only a small subset of 21 members of the GH95 cluster III display this reduced loop size, although most of them possess shorter loops lacking the GGYGYG residue region (FIG. 28, FIG. 32). This major change in this loop is not observed in any of the previously characterized GH95 enzymes. Apart from potential substrate binding, the reduction of this loop is expected to elicit a more open active site pocket of the enzyme and it may contribute to the architecture of the active site and an additional potential substrate binding site on loop 4 (see below). Loop 3 (residues 428-455) contains the tryptophan, which is potentially involved in the recognition (W453) of the galactose at the +1 subsite, and the conformation of this loop is unique in AmGH95B compared to any of the previously determined structures of GH95 enzymes (FIG. 32). This conformational change in loop 3 is possible because of the aforementioned reduction in loop 2, since the extended α-helix in this loop, which is observed in all previous GH95 structures, would block the conformational change of loop 3. Another difference is observed in loop 4 (residues 529-550 of SEQ ID NO.: 14), which contains the conserved SPE motif harboring the general acid catalyst, E541. In cluster III of the phylogenetic tree, the SPE motif follows an aromatic residue (Y: 59.7%, W: 15.5%, F: 15.5%) (FIG. 33). In AmGH95B, this aromatic residue is W538, which is surface exposed due to the deletions in the previously mentioned loops, and close (ca 7 A) to the active site. Adjacent to W538 is an arginine from the same loop (R544, AmGH95B numbering), also conserved in GH95 cluster III and cluster V, which contributes to the positive surface potential around this area and may contribute to substrate recognition with a strong polar interaction. Indeed, AmGH95B, has several positive residues around the surface of this region around loops 3 and 4, including R442 and K514, none of which are conserved in the cluster. The addition of these extra positive amino acids and exposed aromatic residues correlates to the efficiency of AmGH95B on conjugated H type antigens at the surfaces of RBCs.

In a preferred embodiment, the α-1,2-fucosidase according to the invention belongs to the same phylogenetic cluster III of the GH95 enzyme family as AmGH95B. In a preferred embodiment, the α-1,2-fucosidase has a catalytic domain, wherein the amino acid sequence of the domain has 31%, preferably at least 42% sequence identity to amino acids residues 358-730 of SEQ ID NO.: 14.

In a preferred embodiment the α-1,2-fucosidase also has a pI value >7.5.

In a preferred embodiment, the α-1,2-fucosidase is one that can cleave H type 1 pentaose and H type 3 antigens on RBC surfaces.

In a more preferred embodiment, the α-1,2-fucosidase is one that can cleave H type 3 antigen and on the H type 1 pentaose with a normalised rate of >15 s⁻¹ (Table 2).

In an even more preferred embodiment, the α-1,2-fucosidase is able to cleave the terminal non-reducing α-Fucose unit from A type blood within 30 min at room temperature at a concentration of up to 0.2 μM in the conversion buffer (FIG. 30).

### II.vi β-1,3-Galactosidase

The β-1,3-galactosidase belongs to the GH35 enzyme family, preferably an orthologue that populates the same phylogenetic cluster IV as AmGH35A (FIG. 34). AmGH35A comprises a catalytic (β/α)₈ barrel domain followed by a galactose binding-like domain. The amino acid sequence of residues 26-379 SEQ ID NO.: 16 of AmGH35A comprising said catalytic domain shares 38.71% amino acid sequence identity of the catalytic domain of AmGH35B (FIG. 35). Although both AmGH35A and AmGH35B displayed similar catalytic activity on disaccharides (FIG. 36), only AmGH35A was able to convert Gal-A on RBCs (FIG. 37).

In a preferred embodiment, the β-1,3-galactosidase according to the invention has a catalytic domain, wherein the amino acid sequence of said domain has at least 43% sequence identity to the catalytic domain of residues 26-379 SEQ ID no. 16 of AmGH35A.

In a preferred embodiment, the β-1,3-galactosidase also has a pI value >7.5.

In a preferred embodiment, the β-1,3-galactosidase is specifically active on a Gal-β-1,3-linkage to a HexNAc at subsite+1, e.g. active on galacto-N-biose and lacto-N-biose I, but inactive or negligibly active on galactobioses of different linkages. The β-1,3-galactosidase is also preferably one that is active on the extended Gal-A antigen.

In a more preferred embodiment, the β-1,3-galactosidase shows activity against galacto-N-biose (GNB), and is able to fully digest 2 μM of in 1 hour under assay conditions described herein.

Even more preferably, the β-1,3-galactosidase is one that can cleave the terminal non-reducing α-Galactose unit from A type blood within 30 min at room temperature at a concentration of up to 2 μM in the conversion buffer (FIG. 37).

II.vii α-1,3-N-Acetylgalactosaminidase, α-1,2-Fucosidase and β-1,3-Galactosidase

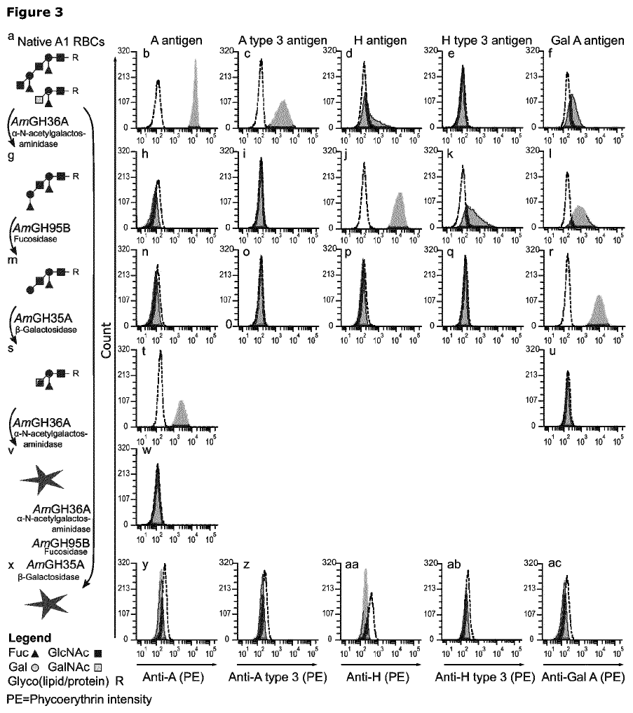

A composition comprising the enzymes of II.iv, II.v and II.vi having α-1,3-N-acetylgalactosaminidase, α-1,2-fucosidase and β-1,3-galactosidase activity can be used to remove extended A antigens and A-antigens from A-type RBCs and deplete these antigens in the enzyme-converted cells, as illustrated in Examples 3.iv and 4 (FIGS. 3-4). Furthermore, a composition comprising the enzyme of II.iv can be used to remove A-antigens from A-type RBCs.

Optionally, the components of said composition may comprise the enzymes of each of Iii, IIii, II.iv, II.v and II.vi (having β-1,3-N-acetylgalactosaminidase, α-1,3-galactosidase α-1,3-N-acetylgalactosaminidase, α-1,2-fucosidase and β-1,3-galactosidase activity in order to remove both extended A- and extended B-antigens and both A- and B-antigens from incompatible RBCs and deplete these antigens in the enzyme-converted cells.

III a Method for Removing A-Antigens, B-Antigens and Extended A- and Extended B-Antigens from A or B (or AB) RBCs

In a third embodiment, the invention provides a method for removing A-antigens, B-antigens and extended A- and extended B-antigens from donor RBCs comprising the steps of:
(i) contacting A or B (or AB) RBCs with the composition(s) of (IIiii) and/or (IIvii) of the second embodiment under low ionic strength, isotonic and neutral pH conditions, and as maintained by a conversion buffer (e.g. 200 mM Glycine, 3 mM NaCl) at pH 6.8 at room temperature, for a period sufficient to remove the A-antigens, B-antigens and extended A- and extended B-antigens, and
(ii) depleting the product of step (i) for purified enzymes of said enzyme composition, wherein said composition is capable of removing all detectable A-antigens, B-antigens and extended A- and extended B-antigens from said RBCs; or at least reducing the level of said detectable A-antigens, B-antigens and extended A- and extended B-antigens on said RBCs relative to corresponding untreated RBCs and/or relative to RBCs treated with only A-antigen and/or B-antigen removing enzymes.

Preferably the product of step (i), comprising the enzyme-treated RBCs, is substantially depleted for purified enzymes by washing the product in a buffered isotonic solution with a physiological ionic strength, for example using buffered phosphate saline (βBS), at room temperature.

The removal of targeted antigens is monitored by immunostaining and flow cytometry analysis, a well-established method. The lowest detection levels vary dependent on reagents (antibodies, fluorophores, etc.), protocol and the instrument in use. In general, the method sensitivity is around a 2000 of molecules detected/cell (Zola, 2004) and in theory >5000 fluorophores are required to overcome the intensity of autofluorescence. However, by using antibodies labelled with a bright fluorescent protein, such as the Phycoerythrin (PE) (containing 30 chromophores/protein with a detection at a wavelength where low autofluorescence occur) lower number of molecules/cell can be detected (Mori 2019, doi:10.1093/jb/mvz052). The antibody binding in the staining protocols are also enhanced by using a low-ionic strength phosphate buffer (Diluent pH 7 from Labex of Scandinavia, Helsingborg, Sweden), for increased antibody binding during antibody incubation and washing (Löw et al., 1974). The flow cytometry protocols in use (described herein) are optimized for detection of low antigen levels. Although the detection limit (number of molecules/cells) has not been defined, the A antigen detection protocol used herein detects the low-A-expressing subtypes A₂ and the even weaker subtype Aₓ and other genetically defined ABO subtypes (Hult & Olsson, 2010).

In addition to the above, the more traditional phenotyping method within transfusion medicine may be used to determine the removal of antigens, such as hemagglutination testing (on gel card or in tube). This is also a method dependent on antibody binding and is typically less sensitive than flow cytometry.

The enzyme treatments are stopped by washing in PBS until the enzymes no longer convert the RBCs. The reaction-solutions are centrifuged 5 min at 2000×g and supernatant removed. The RBCs are then resuspended in PBS and centrifuged again, this is then repeated three times and the RBCs are finally resuspended in RBCs storage solution (Cellstab from Bio-Rad when small amounts of enzyme converted RBCs are prepared for laboratory use; if larger amounts of enzyme converted RBCs are prepared for a potential transfusion, then saline, SAG-M or AS-3 is typically used). Elimination of residual converting enzymes can be confirmed by means of enzyme immunodetection or using labelled enzymes.

The RBC conversion procedure is advantageously performed in a low ionic strength, neutral pH, isotonic conversion buffer (e.g., 200 mM Glycine, 3 mM NaCl, pH 6.8) at room temperature (20-24° C.) as shown in FIG. 10m and FIG. 22k-o. Generally poorer enzymatic efficacies seen in PBS buffer, as compared to conversion buffer, may stem from lower enzyme affinities to substrates due to electrostatic screening in PBS buffer. This property of PBS (and corresponding isotonic buffers having a physiological ionic strength) is an important advantage since its use during washing cycles facilitates the removal of the enzymes used to treat RBCs, and thereby avoids potential adverse effects caused by enzyme carry-over. The enzyme-converted RBCs resulting from the method are more suitable for transfusion to a subject in need thereof than RBCs only treated with A-antigen or B-antigen converting enzymes, as judged by the increased compatibility (noted as decreased crossmatch reactivity) observed when the composition/kit described herein is used.

### IV Use of Enzyme Cocktails for RBC Conversion

In a fourth embodiment, the invention provides the use of the compositions of the second embodiment for enzymatic removal of A-antigens, B-antigens and extended A- and extended B-antigens from potentially ABO-incompatible A, B (or AB) RBCs, thereby achieving the conversion of said RBCs into enzyme-converted RBCs according to the first embodiment lacking the antigens that would otherwise elicit a positive cross-match with plasma from an ABO-incompatible recipient.

### V a Population of Enzyme-Converted ABO-Compatible RBCs, or a Blood Product Derived Therefrom According to the First Embodiment for Use as a Medicament.

RBCs or the blood product derived therefrom may be used as a medicament, for example for the purpose of transfusion.

VI a Population of Enzyme-Converted ABO-Compatible RBCs, or a Blood Product Derived Therefrom According to the First Embodiment for Use in Treatment of a Human Suffering from a Lack of Red Blood

RBCs or the blood product derived therefrom may be used in the treatment of a human suffering from a lack of RBCs or blood product derived therefrom, where the RBCs of the present invention can be administered by transfusion to the subject without risk of cross-match-reactivity.

### EXAMPLES

### Example 1: Cloning of Glycoside Hydrolase Genes from Akkermansia Muciniphila and Expression and Purification of their Encoded Enzymes

A. muciniphila is an atypical dedicated mucolytic gut symbiont, which encodes at least 59 glycoside hydrolases (GHs). As such, A. muciniphila was a predicted potential source of enzymes that can degrade mucin O-glycans and related glyco-conjugates in the mucin O-glycoprotein layer that coats the epithelial enterocytes that shed into the lumen of the gut, and on the surface glyco-conjugates of these cells.

Based on A. muciniphila genomic analyses, genes encoding enzyme with potential activities against both A and B antigens and extended forms thereof were cloned as follows:
1.i Methods: Gene fragments encoding enzymes lacking their signal peptides as predicted using SignalP v. 4.119 from Akkermansia muciniphila ATCC BAA-835 (DSM 22959) were amplified from genomic DNA using sequence specific primers. The amplicons were inserted into the pET28a(+) vector (Novagen, Darmstadt, Germany) within the EcoRI and Ncol sites using In Fusion Cloning (Takara, Shiga, Japan). The recombinant enzymes were designed as fusions with C-terminal His6-tags and expressed in E. coli BL21 (DE3) (Novagen). Cultures were grown to OD₆₀₀ 0.6 before induction with IPTG to 0.1 mM and continued growth at 18° C. for 24 hours. Pellets were harvested by centrifugation (10.000 g, 30 min) and stored frozen at −20° C., until further use. Pellets were re-suspended in binding buffer (20 mM NaPO4, 500 mM NaCl, 10 mM Imidazole, 10% glycerol, pH 7.5) and lysed by one passage through a high-pressure homogenizer (Stansted ‘Pressure Cell’ Homogenizer SPCH Model FPG12800, Essex, UK) at 1000 bar, or ultrasound sonification (QSonica, Newtown, CT, USA) at 40% amplitude (4 min; 20 s on, 40 seconds off). The cell lysates were treated with Benzonase (Novagen, Damstadt, Germany) for 1 hour at 4° C., and then centrifuged (30.000 g, 30 min, 4° C.) to remove cell debris. Next, the recombinant enzymes were purified by His-affinity chromatography using 5 mL HisTrap™ columns (Cytiva, Uppsala, Sweden) installed on an Akta Avant (Cytiva) using a standard protocol. The eluted fractions were pooled, concentrated using a using Amicon Ultra-15 centrifugal filter units (Merck, Burlington, MA, USA) with a molecular weight cutoff of 30 kDa, or 10 kDa depending on enzyme size, and further purified by loading onto a size-exclusion chromatography Hiload 16/60 pg200 column (Cytiva) and eluting at 0.5 mL min-1. Pure elution fractions were collected and concentrated as above. Enzyme concentrations were determined using a Nanodrop (Thermo Fisher, Waltham, MA, USA) and the theoretically determined molar extinction coefficients ε280 nm. The purity and size of the enzymes were assessed by SDS-PAGE analysis.
1.ii Cloned and expressed genes: The following genes were cloned and expressed providing a source of highly efficient enzymes that collectively were able to convert all target ABO antigens:

TABLE 4
Overview of cloned enzymes for the conversion
of all target ABO antigens.

| Enzyme | Locus tag | Genbank accession | SEQ ID NO |
|---|---|---|---|
| AmGH20A | Amuc_0369 | ACD04208 | 2 |
| AmGH20C | Amuc_0868 | ACD04701 | 4 |
| AmGH20I | Amuc_2019 | ACD05829 | 6 |
| AmGH110A | Amuc_0480 | ACD04318 | 8 |
| AmGH36A | Amuc_0216 | ACD04059 | 10 |
| AmGH109B | Amuc_0017 | ACD03864 | 12 |
| AmGH95B | Amuc_1120 | ACD04946 | 14 |
| AmGH35A | Amuc_0771 | ACD04606 | 16 |
| AmGH36C | Amuc_0855 | ACD04688 | 36 |
| AmGH109A | Amuc_0920 | ACD04752 | 38 |
| AmGH95A | Amuc_0186 | ACD04030 | 40 |
| RiFuc95 | | EEG94249 | 42 |

### Example 2: Enzymes for the Conversion of the B Antigen and Extended GalNAc B Antigen

A. muciniphila is shown to be a source of genes encoding enzymes having α-1,3-galactosidase and β-1,3-N-acetylgalactosaminidase activity that in combination are capable of converting both B antigens and extended B antigens on RBCs.
2.i A. muciniphila genome mining for genes encoding B antigen converting enzymes: A search of the genome of A. muciniphila revealed the following potential α-1,3-galactosidases for converting a B-antigen to the universal H antigen (FIG. 12):
Two A. muciniphila genes (Amuc_0480 and Amuc_1463) encode GH110 orthologues that share <33% sequence identity with the B antigen cleaving α-(1,3)-galactosidase FragA (Liu et al. 2007). AmGH110A possesses an N-terminal carbohydrate binding module assigned into CBM51, which harbors members with affinity for galactose and the A/B-antigens, while AmGH110B only possesses a single catalytic module, in common with FragA.

A GH27 candidate gene, Amuc_1187, encodes AmGH27 that shares 36.78% sequence identity in the (β/α)₈ barrel domain with a α-galactosidase from Coffea arabica (Goldstein et al., 1982), a B-antigen converting enzyme; and 30.23% sequence identity to the A antigen converting α-N-acetylgalactosaminidase GH27 fungal orthologue from Acremonium sp. (Kadowaki S, 1989).

Three candidate GH36 α-galactosidase genes, Amuc_0216 (AmGH36A), Amuc_0517 (AmGH36B) and Amuc_0855 (AmGH36C) were assigned to different clades in the phylogenetic tree of characterized GH36 members. The three encoded GH36 enzymes were highly diverse with respect to their size (domain organization) and amino acid identity, having <21% shared sequence identity in the GH36 catalytic (β/α)₈ barrel domain. The GH36 enzyme FpGalNAse is a galactosaminidase, able to convert group A RBCs in a two-step reaction, in combination with a de-acetylase.
2.ii Identification of A. muciniphila enzymes for B-antigen type 2 hexasaccharide conversion: The candidate α-galactosidase enzymes (of 2.i, Table 1, Table 4) were purified and their activities were evaluated on a B-antigen type 2 hexasaccharide using thin layer chromatography (TLC) as follows: 1 μL of the enzymatic reactions was spotted onto silica gel 60 F454 plates (Merck, Burlington, MA, USA) at varying time points. The sugars were separated by a mobile phase consisting of butanol: ethanol: water (5:3:3) (all v/v). For visualization, the plates were sprayed with a solution of 5-methylresorcinol monohydrate: ethanol: sulfuric acid (2:80:10) (all v/v) and subsequent tarring at 570°.

AmGH110A at 1 μM (81.6 μg mL⁻¹) was shown to convert 2 mM B hexasaccharide within 5 min; while neither AmGH110B nor AmGH27 was active on this substrate (FIG. 15, a-c). AmGH36C was inactive the B hexasaccharide than AmGH110A, while the α-GalNAcase AmGH36A had poor activity and AmGH36B no activity on this substrate (FIG. 15, d-f).

The reaction rates of AmGH110A against the B type 2 hexasaccharide at low enzyme concentrations (20 nM) were determined by high-performance anion exchange chromatography with pulsed amperometric detection (HPAEC-PAD). The enzymatic reaction was quenched at various time intervals of 30 seconds to 10 minutes by adding 30 μl of the reaction mixture with 90 μl of 0.1 M NaOH, and the quenched reactions centrifuged (10.000 g, 10 min, 4° C.) and the supernatants were monitored for the release of galactose from the B-antigen substrate. The concentration of the analyte sugar was determined from the area underneath the corresponding peaks, as a function of the concentrations of the standard using Chromeleon (Dionex, Synnyvale, CA, USA). As seen in FIG. 15g-h, the AmGH110A initial reaction rates for conversion of B type 2 hexasaccharide is linear.
2.iii A. muciniphila genome mining for genes encoding extended B antigen converting enzymes: A search of the A. muciniphila genome amongst genes encoding the CAZy enzyme repertoire (CAZyome) identified the GH20 family as the most represented in the A. muciniphila CAZyome, comprising 11 candidate β-N-acetylhexosaminidases (β-HexNAcase) (Table 2, Table 4). The candidates exhibited high sequence divergence with 5 candidates featuring additional unannotated putative domains.
2.iv Identification of A. muciniphila enzymes for extended B-antigen type 2 hexasaccharide conversion: To identify a β-N-acetylgalactosaminidase active on the GalNAc-B antigen, genes encoding each of the candidate GH20 enzymes from A. muciniphila were cloned, expressed, and purified. All GH20 but one (AmGH20K) exhibited β-N-acetylgalactosaminidase activity on both pNP-β-GalNAc and pNP-β-GlcNAc substrates (Table 5), which is desired for the removal of GalNAc-B extension. Notably, AmGH20A exhibited the highest reaction rates for both analogues.

TABLE 5
Reaction rates of enzymes against Aryl substrate analogues selected
as candidates for conversion of extended GalNAc B antigens.

| Enzyme | Normalized rate | (V₀/E) (s⁻¹)* | SEQ ID NO |
|---|---|---|---|
| | pNP-β-GalNac | pNP-β-GlcNAc | |
| AmGH20A | 60.8 ± 5.7 | 109.7 ± 11.7 | 2 |
| AmGH20B | 0.31 ± 0.20 | 0.16 ± 0.08 | 18 |
| AmGH20C | 8.3 ± 2.9 | 31.3 ± 11.1 | 4 |
| AmGH20D | 0.06 ± 0.03 | 0.44 ± 0.32 | 20 |
| AmGH20E | 2.1 ± 0.6 | 4.6 ± 0.8 | 22 |
| AmGH20F | 0.29 ± 0.17 | 0.05 ± 0.03 | 24 |
| AmGH20G | 8.0 ± 5.6 | 0.05 ± 0.03 | 26 |
| AmGH20H | 0.17 ± 0.09 | 2.3 ± 1.2 | 28 |
| AmGH20I | 4.8 ± 1.9 | 18.16 ± 6.53 | 6 |
| AmGH20J | 0.3 ± 0.2 | 45.2 ± 33.4 | 30 |
| AmGH20K | (0.9 ± 0.1) · 10⁻³ | (0.25 ± 0.14) · 10⁻¹ | 32 |
| | pNP-β-Gal | pNP-β-GalNAc | |
| AmGH35A | (8.6 ± 0.04) · 10⁻¹ | N.D. | 16 |
| AmGH35B | 21.9 ± 3.9 | N.D. | 44 |

*Each enzyme was assayed in 20 mM Sodium Phosphate, 150 mM NaCl, 0.005% (v/v) Triton X-100, pH 7.0. The release of p-nitrophenol was monitored by the increase in A₄₁₀ over time. The initial reaction rates (Vₒ) were determined from the linear part of the A₄₁₀ versus time and pNP standard curves (0.02-3 mM). The reaction rates were normalized by dividing with the enzyme concentration (Vₒ/E) (s⁻¹). Activity data are from n = 3 independent experiments

2.v A. Muciniphila Enzymes Efficiently Remove B Antigen and Extended GalNAc B Antigen from RBCs

Native RBCs mainly present the B antigen, but may present the extended B antigen, whose conversion by A. muciniphila enzymes was demonstrated as follows:
Methods: Packed RBCs (pRBCs) A 1 volume was primed in 4× volume conversion buffer (200 mM glycine, 3 mM NaCl, pH 6.8), for 15 min while mixing, followed by centrifugation for 5 min at 1000×g. The supernatant was removed and the RBCs were diluted in 6× volume buffer and pelleted again. The respective enzyme was diluted in conversion buffer and the pRBCs were mixed with x1.6 enzyme solution, and shaken at room temperature for 30 minutes. The reaction was stopped by washing the cells in PBS four times. The cells were finally diluted in ID-cellstab (Bio-Rad) and stored at 4° C. The removal of the type B antigens and extended B antigens was detected by immunostaining of RBCs and flow cytometry analysis. An anti-B antigen monoclonal and natural anti-GalNAc-B antibodies from blood donors were used to distinguish the B antigen and extended B antigen employing a protocol optimized to detect low levels of the antigen (Hult and Olsson 2010).
B antigen conversion: AmGH110A successfully removed the B antigen of the RBCs in conversion buffer (200 mM glycine, 3 mM NaCl, pH 6.8), whereby 0.05 μM enzyme was sufficient to remove detectable B antigens on RBCs within 30 min (FIG. 2A and/or 2B, FIG. 14). While AmGH36A caused some decrease of B antigen levels under the same conditions, AmGH36C and AmGH27 had no detectable effect on the targeted antigen (FIG. 14)
Extended B antigen conversion: Only AmGH20A, AmGH20C and AmGH20I out of the 11 enzyme panel (AmGH20A-K), showed efficient conversion of extended B antigens on RBCs after 30 min using 1 μM of each enzyme (FIG. 10 a-f). However, using lower enzyme concentrations (0.02-2 μM), AmGH20C was found to be the most efficient GalNAc-B antigen converting enzyme on RBCs (FIG. 10 g-k). Neither AmGH20A and AmGH20C were active on the GalNAc-B antigen on RBCs in PBS buffer (FIG. 10 l-m). Accordingly, AmGH110A can be used for conversion of B antigens on RBCs treatment, while one-pot treatment can be used for conversion of both B antigens and extended B antigens with either a combination of AmGH110A and AmGH20A (FIG. 2A) or AmGH110A and AmGH20C (FIG. 2B).
   2vi: RBCs Treated with the AmGH110A and AmGH20A are Less Crossmatch Reactive with Group O Donor Plasmas Compared to RBCs Treated with Only AmGH110
Methods: Serologic testing was conducted according to standard blood banking practice in tube or gel cards (Bio-Rad). The reactions were read as negative (−), positive, grading from one plus (1+) to four plus (4+) or mixed field. For confirming or further blood group typing monoclonal anti-B (9621A8, Diagast, Loos, France), anti-Le^{a} (GAMA701, Immucor, Norcross, GA, USA) and anti-Leb (GAMA704, Immucor) was used. The compatibility of the enzyme treated RBCs with other donors' plasma was subjected to crossmatching. RBC suspensions (50 μl) at 0.8% in low ionic storage solution (ID-cellstab) was mixed with 25 μl plasma and incubated 15 min at 37° C. on IgG gel cards (Bio-Rad) followed by centrifugation, 10 min at 85×g.

To evaluate how removal of the extended GalNAc-B antigen structure and B antigen compared to only removing the B antigen, in terms of compatibility a crossmatch screen with O plasmas (n=100) was conducted. Group B RBCs from one secretor (expected to carry B types 1-4 core structure) and one non-secretor (lacking the core 1 structures of the B antigen on RBCs compared to secretor), (FIG. 5). The secretor B RBCs are already post AmGH110A (0.5 μM) treatment nonreactive with a majority of O plasmas (79%), (FIG. 5a). However, post treatment with AmGH110A (0.5 μM) and AmGH20A (0.4 μM) negative reactivity reaches 91%. The median reactivity strength had a significant difference between the two treatments. Additionally, after AmGH110A (0.5 μM) and AmGH20C (0.4 μM) one-pot treatment compared to the single treatment negative reactivity reached 84% plasmas as compared to 79% with AmGH110A alone (FIG. 5a). The mean reactivity values are seen in the graph of FIG. 5b, as well as those for the non-secretor RBCs. Furthermore, the RBCs of the different treatments were tested with AB (n=11) and A (n=12) plasmas. The AB plasmas (FIG. 5c) were negative with both native RBCs as well as all types of enzyme treated RBCs, while the A plasmas (FIG. 5d) reacted (as expected) with the native B RBCs and one A₂ plasma reacted with the AmGH110A (0.5 μM) treated secretor RBC. The native non secretor RBCs reacted as expected with AB and A plasmas, while the treated RBCs were all negative (FIG. 5d).

### Example 3: Enzymes for the Conversion of an a Antigen and Extended a Antigen

### 3.i Mining for A. Muciniphila Genes Encoding a Antigen Converting Enzymes

The genome of A. muciniphila encodes two α-N-acetylgalactosaminidases of the glycoside hydrolase family 109 (AmGH109A and AmGH109B), which share about 36% identity with the previously described A antigen-cleaving orthologue NagA (Lui et al., 2007). AmGH36A was selected based on observed α-GalNAcase activity of this enzyme. Their respective domain organization and sequence identities are shown in FIG. 21, and their predicted properties in Tables 6.

TABLE 6
Predicted properties of A. muciniphila enzyme candidates
for A and extended A antigen conversion

| Target | | SP/Construct | Mw | ε₂₈₀ | |
|---|---|---|---|---|---|
| epitope | Enzyme | Start (AA) | (kDa) | M⁻¹ cm⁻¹ | pI^{b} |
| A/A type 3 | AmGH109A | 29 | 48.8 | 1.44 | 6.78 |
| | AmGH109B | 31 | 51.6 | 1.78 | 8.46 |
| Gal-A | AmGH35A | 24 | 66.2 | 1.95 | 9.06 |
| | AmGH35B | 17 | 86.5 | 1.64 | 8.70 |
| H type 3 | AmGH95A | 23 | 84.7 | 1.44 | 8.90 |
| | AmGH95B | 13 | 87.1 | 2.02 | 8.94 |

Size of the signal peptide regions as predicted by SignalP (V.6.0) and deepTMHMM (V.1.0.20). The first amino acid of the final recombinant enzymes starts at the next amino acid following the signal peptides. The computed properties in the table are based on the amino acid sequence of recombinant enzymes that comprise the mature peptides lacking signal peptides fused to a hexa-histidine C-terminal tags, with the extinction coefficient assuming that all cysteine residues are reduced.

### 3.ii Identification of A. Muciniphila Enzymes for a Antigen Conversion:

The candidate enzymes (of 3.i, Table 3, Table 6) were purified and their activities were evaluated on A type 2 antigen hexaose using thin layer chromatography (TLC) as described in 2.ii. AmGH36A was the most efficient enzyme, where 20 nM enzyme converted 2 mM of the B hexaose within 10 minutes. AmGH109A, followed by AmGH109B, was the least efficient, where 1 μM enzyme was only able to fully convert 2 mM of the A hexaose within 1 hour. The activity of AmGH36A against the A type 2 antigen hexaose, is a prerequisite and consistent with its activity against the structurally similar extended A type 3 antigen (FIG. 1).

### 3.iii Identification of A. Muciniphila Enzymes for Extended a Antigen Conversion:

A. muciniphila comprises genes encoding two α-1,2-fucosidases, AmGH95A and AmGH95B. The two enzymes were expressed and purified. AmGH95B (20 mM) demonstrated an extraordinary efficiency on the H type 1 pentaose, completely degrading the 2 mM oligosaccharide within 10 minutes, while AmGH95A was inactive on this oligosaccharide. The activity of AmGH95B against the H type 1 pentaose, is consistent of its activity against the structurally similar extended H type 3 antigen (FIG. 1)

A. muciniphila possesses two members of the GH35 family, which is reported to contain β-1,3-galactosidases, where AmGH35B harbour a CBM32 ancillary domain (FIG. 35). Both enzymes were expressed, purified and shown to be β-(1,3)-galactosidases based on their activity against both para-nitrophenyl-β-galactopyranosides (Table 5, FIG. 36), but having no activity on β-(1,4/6) linkages. The activity of AmG35A activity against the T-antigen disaccharide (also called Galacto-N-biose β-1,3-galactosides, is a potential signature for its activity against the structurally similar extended Gal-A antigen (FIG. 1).

### 3.iv the Three Known Extensions and the Core a Antigen on RBCs are Efficiently Removed by Three A. Muciniphila Enzymes

Conversion of all A antigen including the A type 3 extended antigen: AmGH36A was able to both convert the A antigen on RBCs carried on RBCs from both the dominant A₁ phenotype (high relative expression of A antigens on the RBC surfaces) and the less common A₂ phenotype (lower expression of A antigens relative to A₁) after 30 min in conversion buffer, including the A type 3 extended antigen on RBCs, based on flow cytometry analysis (FIG. 3 and FIG. 22).

Methods: Packed RBCs (pRBCs) in 1 volume was primed in 4× volume conversion buffer (200 mM glycine, 3 mM NaCl, pH 6.8), for 15 min while mixing, followed by centrifugation for 5 min at 1000×g. The supernatant was removed and the RBCs were diluted in 6× volume buffer and pelleted again. The respective enzyme was diluted in conversion buffer and the pRBCs were mixed with x1.6 enzyme solution, and shaken at room temperature for 30 minutes. The reaction was stopped by washing the cells in PBS. The cells were finally diluted in ID-cellstab (Bio-Rad) and stored at 4° C. The removal of the type A antigens and extended A antigens was detected by immunostaining of RBCs and flow cytometry analysis. RBCs were stained with the Abs anti-A (ES-15), anti-B (9621A8) and anti-H (BRIC231) followed by RAM-κ:PE as described by Hult and Olsson (2010). The same procedure was used for staining with the Abs anti-T/Gal-A (3C9), anti-A type 3 (TH1) and anti-H type 3 (HH14), although the RBCs were papain-treated before staining to increase sensitivity.

Conversion of H type 3 extended antigen: Three α-1,2-fucosidases were tested, RiGH95, AmGH95A and AmGH95B from A. muciniphila. AmGH95B was extremely efficient in conversion of the H type 3 antigen, while AmGH95A and RiGH95 had very low/no activity, which was confirmed in treatment of A₁ RBCs pretreated with AmGH36A (FIG. 3g-r, FIG. 22).

Conversion of Gal-A antigen: RBCs pretreated with AmGH36A and then AmGH95B led to the accumulation of the extended Gal-A antigen, which requires β-1,3-galactosidase for its removal. AmGH35A, but not AmGH35B, was capable of reducing the Gal-A antigen on A₁ RBCs both without pretreatment with other enzymes (FIG. 3, FIG. 37), or RBCs pretreated with AmGH36A and AmGH95B, which results in higher relative concentration of this antigen, as compared to the non-treated cells (FIG. 3 m-u).

Lastly, the sequential digests of A₁ RBC carried antigens, conducted by stepwise incubations with AmGH36A, AmGH95B, AmGH35A reveals additional A antigens, which required a last step treatment with AmGH36A to generate RBCs negative for the A antigen (FIG. 3 s-w).

### Example 4: RBCs Treated with the AmGH36A, AmGH95B and AmGH35A are Less Crossmatch Reactive with Group O Donor Plasmas Compared to RBCs Treated with Only AmGH36A—Proving the Antigenicity of a Extended Antigens

Cross-match test is a hemagglutination method used in the clinical practice to avoid incompatible transfusions.

### 4.i Methods:

Serologic testing was conducted according to standard blood banking practice in tube or gel cards (Bio-Rad). The reactions were read as negative (−), positive, grading from one plus (1+) to four plus (4+) or mixed field. For confirming or further blood group typing monoclonal anti-A (A003, Seraclone/Bio Rad, Hercules, CA, US), anti-H (lectin of Ulex Europaeus, Immucor), anti-Le^{a} (GAMA701, Immucor, Norcross, GA, USA) and anti-Le^{b}(GAMA704, Immucor) was used. Similar conclusions were valid for RBC conversion and performing a similar series of crossmatches (FIG. 5).

The compatibility of the enzyme-treated RBCs with other donors' plasma was subjected to crossmatching at 37° C. on IgG gel cards. The A₁ RBCs in the sequential treatments according to FIG. 3, were crossmatched with plasma from three group O donors. One plasma was known to be highly reactive with ABO antigens, while two were randomly picked. All three plasmas reacted as expected strongly (4+) with the native A₁ RBCs. The reactivity then changed individually for each plasma post treatments, displaying the diversity of different donors' antibody-cocktail in their plasma (data not shown). Importantly, the plasma reactivities with RBCs subjected to all treatment-steps, AmGH36A→AmGH95B→AmGH35A→AmGH36A, were markedly decreased. The highest reactive plasma had a weaker reactivity (2+) and the other two plasmas were nonreactive.

To evaluate how the strategy of targeting the A antigen and its extended structures compared to only removing the A antigen, a larger crossmatch screen was conducted with group O plasmas (n=100). The A₁ RBCs from two different donors, one secretor (expected to carry A type 1-4 core structure) and one non-secretor (lacking core 1 carried A) were crossmatched (FIG. 4a). The O plasmas had a median reactivity at 2+ with AmGH36A treated A1 RBCs from both secretor and non-secretor RBC donors (FIG. 4b), where in total only 17 and 21 plasmas, respectively, were nonreactive. The combined treatments with AmGH36A, AmGH95B and AmGH35A of RBCs from the same secretor and non-secretor had a significantly weaker median reactivity at 1+ and negative, respectively. The triple enzyme treated RBCs from the secretor were nonreactive with 48 of the O plasmas and the non-secretor with 52. The strategy was also evaluated by conversion of secretor RBCs and crossmatching with A₁B plasmas, A₂B, as well as B plasmas (FIG. 4c). A single +2 crossmatch with the A₁B was observed for the RBCs' treatment with AmGH36 alone, and this was abolished by treatment with the enzyme combination as above, to get a single weak crossmatch (+1) cross with the A₂B. On the other hand, more then half the plasmas gave crossmatches with the GH36 treatment alone, which was reduced to only three weak crossmatches (+1) out of 12 plasmas. These latter results were fairly similar to the treatment of A₁ (A¹O¹ genotype) cells and crossmatches with B plasmas (FIG. 4d). These findings demonstrate that converting both the A (of all types), H-type 3 and Gal-A antigen on RBCs is an improvement over only removing the A antigen; and confirms that H-type 3 and Gal-A antigens are a part of the A-related antigenicity of the group A phenotype. This further shows the potential for transfusion across the different blood groups of enzyme-converted A RBCs.

### Example 5: Efficient Desorption of Conversion Enzymes from RBCs Obtained by Post-Treatment Washing Cycles in PBS

The level of residual conversion enzymes bound to the surface of RBCs following treatment with conversion enzymes and subsequent washing steps was analyzed using fluorophore labelled enzymes.

5i. Methods:

Fluorophore labelled enzymes were prepared as follows: A. muciniphila enzymes and an α-1,6-glucosidase from Lactobacillus acidophilus (LaGH13_31) as a non-RBC interacting negative control, were each labelled with Sulfo-Cyanine5 NHS ester (Cy5) from Lumiprobe (Hannover, Germany) dissolved in DMSO according to the manufacturer's protocol using a Cy5:enzyme molar ratio of 16:1 and an incubation time of 18 h on ice. The degree of labelling was estimated to be between 0.84-1.98, using a Cy5 standard curve generated using 4 concentrations in the range 0-800 nM and by measuring enzyme concentration at A₂₈₀. The activity of the enzymes was also compared before and after labelling and found to be between 0.7 and 0.9.

The retention of enzymes on treated RBCs was monitored by incubating A or B RBCs with each labelled enzyme for 30 min at room temperature (using 1 μM or 0.5 μM of A-core or B-core enzymes respectively, equivalent to the highest concentration in each one-pot reaction) in conversion buffer, and subsequently washing four times in PBS at room temperature. The same treatment was also performed using an α-1,6-glucosidase (LaGH13_31) as negative control, as based on its activity it was not expected to specifically interact with RBCs. The total fluorescence was measured by incubating the enzymes with conversion buffer alone and the background fluorescence from the RBCs was measured by incubating them in conversion buffer devoid of enzymes, which was used to correct the signal from the reactions. The fluorescence emission was measured on a JASCO FP8500 spectrofluorometer with emission and excitation band width of 10 nM, and an excitation wavelength of 646 nM with medium sensitivity. The emission data were collected using the SpectraManager suite v.2 software supplied with the instrument (program: Fixed Wavelength Measurement) at 662 nM and accumulated average from three data points was reported. The total fluorescence from each step was calculated from multiplying the measured fluorescence mL⁻¹ with the volume of reactions and washes to estimate the fraction of Cy5-labelled enzyme that remains associated with the cells. These experiments were performed in four independent replicates, as shown in FIG. 38.

Flow cytometry was used to measure residual Cy5-labelled AmGH36A and AmGH110A enzymes on A and B RBCs respectively following incubation in conversion buffer and similar washing in PBS to mimic the standard conversion protocol described herein. The Cy5-labelled α-1,6-glucosidase (LaGH13_31, 1 μM) was used as negative control, in addition to the respective non-labelled enzymes. The association of the Cy5-labelled enzymes on RBCs treated with either enzyme alone or with the one-pot B-cocktail and subsequently washed four times with PBS buffer is shown in FIG. 39.

As seen in FIG. 38, fluorescently-labelled conversion enzymes incubated with RBCs were efficiently removed post-conversion by washing in PBS, which served to desorb enzymes from the RBCs. The low levels of fluorescence associated with RBCs after 4-washes were comparable to control RBCs incubated with a non-blood interacting enzyme after 4-washes. Flow cytometry analyses (FIG. 39) of RBCs incubated with fluorescently-labelled conversion enzymes and then washed showed that only a minor proportion of the RBC population remained associated with the fluorophore-labelled enzymes.

### Embodiments of the Invention

A first embodiment provides a population of enzyme-converted ABO-compatible red blood cells having no detectable A-antigens, B-antigens and extended A- and extended B-antigens as determined by serological typing,
wherein said enzyme-converted ABO-compatible red blood cells are obtained by enzymatic removal of A-, B-antigens and one or more of extended A type 3-, H type 3- and Gal-A-extended antigens and extended B (ExtB) antigens on A, B or AB red blood cells, and
wherein said enzymatic removal of said extended B-antigens is mediated by contacting said A, B or AB red blood cells with:
a β-1,3-N-acetylgalactosaminidase belonging to phylogenetic cluster II GH20 family and
an α-1,3-galactosidase belonging to phylogenetic cluster II of GH110 family; and
wherein said enzymatic removal of said extended A-antigens is mediated by contacting said A, B or AB red blood cells with:
an α-1,3-N-acetylgalactosaminidase belonging to phylogenetic cluster I of GH36 family or phylogenetic cluster II of GH109 family, each having an isoelectric point (pI) value of >7.5;
an α-1,2-fucosidase belonging to phylogenetic cluster III of GH95 family and having a pI value of >7.5; and
a β-1,3-galactosidase belonging to phylogenetic cluster IV of GH35 family and having a pI value of >7.5.

In a second embodiment, the population of enzyme-converted ABO-compatible red blood cells of the first embodiment,
wherein said β-1,3-N-acetylgalactosaminidase has a catalytic domain characterized by an amino acid sequence having at least 61% sequence identity to amino acid residues 164-552 of SEQ ID NO.: 2 comprising the catalytic domain of AmGH20A; and
wherein said α-1,3-galactosidase has a catalytic domain characterized by an amino acid sequence having least 36% sequence identity to amino acid residues 213-795 of SEQ ID NO. 8 comprising the catalytic domain of AmGH110A, and
wherein said β-1,3-N-acetylgalactosaminidase and said α-1,3-galactosidase have a pI value of >7.5.

In a third embodiment, the population of enzyme-converted ABO-compatible red blood cells of the first or second embodiment,
wherein said α-1,3-N-acetylgalactosaminidase belonging to phylogenetic cluster I of the GH36 family has a catalytic domain characterized by an amino acid sequence having at least 36% sequence identity to amino acid residues 385-749 of SEQ ID NO.: 10 comprising the catalytic domain of AmGH36A, and said α-1,3-N-acetylgalactosaminidase belonging to phylogenetic cluster II of the GH109 family has a catalytic domain characterized by an amino acid sequence having at least 46% sequence identity to amino acids residues residues 27-481 of SEQ ID NO. 12 comprising the catalytic domain of AmGH109B; and
wherein said α-1,2-fucosidase has a catalytic domain characterized by an amino acid sequence having at least 42% sequence identity to amino acids residues 358-730 of SEQ ID NO.: 14 comprising the catalytic domain of AmGH95B; and
wherein said β-1,3-galactosidase has a catalytic domain characterized by an amino acid sequence having at least 43% sequence identity to amino acids residues 26-379 of SEQ ID NO.: 16 comprising the catalytic domain of AmGH35A, and
wherein said α-1,3-N-acetylgalactosaminidase, α-1,2-fucosidase, and β-1,3-galactosidase all having a pI value >7.5.

A fourth embodiment provides a blood product comprising the population of enzyme-converted ABO-compatible red blood cells according to any one of the first to third embodiment, wherein said cells are reconstituted as a cell suspension in a medium suitable for transfusion.

In a fifth embodiment, the blood product of the fourth embodiment,
wherein said population of enzyme-converted ABO-compatible red blood cells has: a level of detectable A-antigens, B-antigens and extended A- and extended B-antigens on said red blood cells reduced by at least 95% relative to corresponding untreated A or B (or AB) red blood cells, as measured by antibody staining and flow cytometry; or improved compatibility as compared to corresponding untreated A, B or AB red blood cells, or enzyme-converted A, B or AB red blood cells obtained from corresponding untreated A, B or AB red blood cells by enzymatic removal of only A-, B-antigens, wherein said compatibility is tested against a statistically meaningful cohort of group O plasmas.

A sixth embodiment provides the blood product according to the fourth or fifth embodiment, for use as a medicament.

In a seventh embodiment, the blood product according to the sixth embodiment, for use in treatment of a human suffering from anaemia requiring transfusion.

An eighth embodiment provides an enzyme composition or kit for conversion of donor red blood cells to enzyme-converted ABO-compatible red blood cells according to any one of the first to third embodiments, comprising purified enzymes characterized as:
(i) a β-1,3-N-acetylgalactosaminidase belonging to phylogenetic cluster II of GH20 family and having a pI value of >7.5, and
(ii) a α-1,3-galactosidase belonging to phylogenetic cluster II of GH110 family and having a pI value of >7.5;
and/or purified enzymes characterized as:
(iii) a α-1,3-N-acetylgalactosaminidase belonging to phylogenetic cluster I of GH36 family and having a pI value of >7.5 or phylogenetic cluster II of GH109 family and having a pI value of >7.5,
(iv) a α-1,2-fucosidase belonging to phylogenetic cluster III of GH95 family and having a pI value of >7.5 and
(v) a β-1,3-galactosidase belonging to phylogenetic cluster IV of GH35 family and having a pI value >7.5.

In a ninth embodiment, the enzyme composition or kit according to the eighth embodiment,
wherein said β-1,3-N-acetylgalactosaminidase belonging to phylogenetic cluster II of the GH20 family has a catalytic domain characterized by an amino acid sequence having at least 61% sequence identity to amino acid residues 164-552 of SEQ ID NO.: 2 comprising the catalytic domain of AmGH20A, and
wherein said β-1,3-N-acetylgalactosaminidase a pI value >7.5.

In a tenth embodiment, the enzyme composition or kit according to the eighth or ninth embodiment, wherein said α-1,3-galactosidase belonging to phylogenetic cluster II of the GH110 family has a catalytic domain characterized by an amino acid sequence having least 36% sequence identity to amino acid residues 213-795 of SEQ ID NO. 8 comprising the catalytic domain of AmGH110A, and
wherein said α-1,3-galactosidase has a pI value >7.5.

In a eleventh embodiment, the enzyme composition or kit according to any one of the eighth to tenth embodiment, wherein said α-1,3-N-acetylgalactosaminidase belongs to:
(I) phylogenetic cluster I of the GH36 family and has a catalytic domain characterized by an amino acid sequence having at least 36% sequence identity to amino acid residues 385-749 of SEQ ID NO.: 10 comprising the catalytic domain of AmGH36A or
(II) phylogenetic cluster II of the GH109 family and has a catalytic domain characterized by an amino acid sequence having at least 46% sequence identity to amino acids residues residues 27-481 of SEQ ID NO. 12 comprising the catalytic domain of AmGH109B, and
wherein said α-1,3-N-acetylgalactosaminidase has a pI value of >7.5.

In a twelfth embodiment, the enzyme composition or kit according to any one of the eighth to eleventh embodiment, wherein said α-1,2-fucosidase belonging to phylogenetic cluster III of the GH95 family has a catalytic domain characterized by an amino acid sequence having at least 42% sequence identity to amino acids residues 358-730 of SEQ ID NO.: 14 comprising the catalytic domain of AmGH95B, and wherein said α-1,2-fucosidase has a pI value >7.5.

In a thirteenth embodiment, the enzyme composition or kit according to any one of the eighth to twelfth embodiment, wherein said β-1,3-galactosidase belonging to phylogenetic cluster IV of the GH35 family has a catalytic domain characterized by an amino acid sequence having at least 43% sequence identity to amino acids residues 26-379 of SEQ ID NO.: 16 comprising the catalytic domain of AmGH35A, and
wherein said β-1,3-galactosidase has a pI value >7.5.

A fourteenth embodiment provides a method for removing A-antigens, B-antigens and extended A- and extended B-antigens from A, B or AB red blood cells comprising the steps of:
(i) contacting A, B or AB red blood cells with the enzyme composition according to any one of the eighth to thirteenth embodiment under low ionic strength, isotonic and neutral pH conditions at room temperature for a period sufficient to remove the A-antigens, B-antigens and extended A- and extended B-antigens, and
(ii) depleting the product of step (i) for the purified enzymes of said enzyme composition,
wherein said enzyme composition is capable of removing all detectable A-antigens, B-antigens and extended A- and extended B-antigens from said red blood cells.

In a fifteenth embodiment, the method of the fourteenth embodiment, wherein the product of step (i) is depleted for the purified enzymes of said enzyme composition by washing said product in phosphate buffered saline at room temperature.

In a sixteenth embodiment, the method according to the fourteenth or fifteenth embodiment, wherein red blood cells present in the blood product obtained in step (ii) are recovered and reconstituted as a cell suspension in a medium suitable for transfusion.

A seventeenth embodiment provides the use of the composition or kit according to any one of the eighth to thirteenth embodiment for enzymatic removal of A-antigens, B-antigens and extended A- and extended B-antigens from group A, B or AB red blood cells to obtain ABO-universal, compatible red blood cells.

An eighteenth embodiment provides a method for treating a human in need thereof by administering the blood product according to the fourth or fifth embodiment to said human.

In a nineteenth embodiment, the method of treating a human suffering from anaemia requiring transfusion by administering the blood product according to the fourth or fifth embodiment to said human.

### REFERENCES

Drula. E. (2022) The carbohydrate-active enzyme database: functions and literature. Nucleic Acids Res 50: D571-D577. doi: 10.1093/nar/gkab1045
Yin Y (2012) dbCAN: a web resource for automated carbohydrate-active enzyme annotation, Nucleic Acids Res. (Web Server issue): W445-51. doi:10.1093/nar/gks479.
Paysan-Lafosse T, (2022) InterPro in 2022. Nucleic Acids Research, Vol. 51 D418-D427 (doi: 10.1093/nar/gkac993)
Almeida A, (2021) A unified catalog of 204,938 reference genomes from the human gut microbiome, Nature Biotechnology, Vol 39, 105-114. doi: 10.1038/s41587-020-0603-3
Clausen et al., (1985), Repetitive A epitope (type 3 chain A) defined by blood group A1-specific monoclonal antibody TH-1: chemical basis of qualitative A1 and A2 distinction Proc. Natl. Acad. Sci. USA 82 (4) 1199-1203; doi: 10.1073/pnas.82.4.1199.
Clausen et al., (1986) Novel blood group H glycolipid antigens exclusively expressed in blood group A and AB erythrocytes (type 3 chain H). I. Isolation and chemical characterization, J Biol Chem, I 25; 261(3):1380-7 (βMID: 3944091) and II (βMID: 3944092))
Clausen et al., (1986) Novel blood group H glycolipid antigens exclusively expressed in blood group A and AB erythrocytes (type 3 chain H). II. Differential conversion of different H substrates by A1 and A2 enzymes, and type 3 chain H expression in relation to secretor status, J Biol Chem. 25; 261(3):1388-92.
Clausen et al., (1988) Monoclonal antibodies directed to the blood group a associated structure, galactosyl-A: Specificity and relation to the thomsen-friedenreich antigen. Molec. Immunol. 25(2): 199-204; DOI: 10.1016/0161-5890(88)90068-5
Giarratana et al., (2011) Proof of principle for transfusion of in vitro-generated red blood cells in Blood 118(19):5071-9. doi: 10.1182/-2011-06-362038.
Goldstein, J., Siviglia, G., Hurst, R., Lenny, L. & Reich, L. (1982) Group B Erythrocytes Enzymatically Converted to Group O Survive Normally in A, B, and O Individuals Published by: American Association for the Advancement of Science Stable URL: http://www.jstor.org/stable/1687589. Science (80-.). 215, 168-170.
Hult and Olsson (2010) Many genetically defined ABO subgroups exhibit characteristic flow cytometric patterns. Transfusion 50, 308-323, DOI: 10.1111/j.1537-2995.2009.02398.x.
Judd (1999) Elution-Dissociation of antibody from red blood cells: Theoretical and practical considerations; Transfusion Medicine Reviews 13(4): 297-310 DOI: 10.1016/s0887-7963(99)80059-5.
Liu, Q. P. et al. (2007) Bacterial glycosidases for the production of universal red blood cells. Nat. Biotechnol. 25, 454-464.
Löw et al., (1974) Antiglobulin test in low-ionic strength salt solution for rapid antibody screening and crossmatching Vox Sang. 26(1):53-61. doi: 10.1111/j.1423-0410.1974.tb02666.x.
Mori et al., (2019), Signal amplification in flow cytometry for cell surface antigen analysis The J Biochem 166(3):205-212; doi:10.1093/jb/mvz052
NCBI; The National Center for Biotechnology Information (NCBI) Reference Sequence (RefSeq)databaseNCBI” https://www.ncbi.nlm.nih.gov/refseq/about/nonredundantproteins/
Kadowaki, S. (1989) Isolation and Characterization of a Blood Group A Substance-degrading α-N-Acetylgalactosaminidase from an Acremonium sp., Agricultural and Biological Chemistry, Vol 53, 111-120. doi: 10.1271/bbb1961.53.111
Trakarnsanga et al., (2017) An immortalized adult human erythroid line facilitates sustainable and scalable generation of functional red cells, in Nat Commun 8:14750. doi: 10.1038/ncomms14750.
Valeri C R et al., (2008) The Effects of Preserved Red Blood Cells on the Severe Adverse Events Observed in Patients Infused with Hemoglobin Based Oxygen Carriers, in Artificial Cells Blood Substitutes and Biotechnology (formerly known as Artificial Cells Blood Substitutes and Immobilization Bi 36(1):3-18, DOI: 10.1080/10731190701857736
Zola (2004) High-sensitivity immunofluorescence/flow cytometry: detection of cytokine receptors and other low-abundance membrane molecules. Curr Protoc Cytom. Chapter 6:Unit 6.3. doi: 10.1002/0471142956.cy0603s30.

1. A population of enzyme-converted ABO-compatible red blood cells having no detectable A-antigens, B-antigens and extended A- and extended B-antigens as determined by serological typing,
wherein said enzyme-converted ABO-compatible red blood cells are obtained by enzymatic removal of A-, B-antigens and one or more of extended A type 3-, H type 3- and Gal-A-extended antigens and GalNAc-extended B-antigens on A, B or AB red blood cells,
wherein said enzymatic removal of said extended B-antigens is mediated by contacting said A, B or AB red blood cells with:
a. a β-1,3-N-acetylgalactosaminidase belonging to phylogenetic cluster II GH20 family and having a catalytic domain characterized by an amino acid sequence having at least 61% sequence identity to amino acid residues 164-552 of SEQ ID NO.: 2 comprising the catalytic domain of AmGH20A; and
b. an α-1,3-galactosidase belonging to phylogenetic cluster II of GH110 family and having a catalytic domain characterized by an amino acid sequence having least 36% sequence identity to amino acid residues 213-795 of SEQ ID NO. 8 comprising the catalytic domain of AmGH110A;
wherein said β-1,3-N-acetylgalactosaminidase and said α-1,3-galactosidase have a pI value >7.5; and
wherein said enzymatic removal of said extended A-antigens is mediated by contacting said A, B or AB red blood cells with:
c. an α-1,3-N-acetylgalactosaminidase belonging to:
phylogenetic cluster I of GH36 family and having a catalytic domain characterized by an amino acid sequence having at least 36% sequence identity to amino acid residues 385-749 of SEQ ID NO.: 10 comprising the catalytic domain of AmGH36A; or
phylogenetic cluster II of GH109 family and having a catalytic domain characterized by an amino acid sequence having at least 46% sequence identity to amino acids residues residues 27-481 of SEQ ID NO. 12 comprising the catalytic domain of AmGH109B;
d. an α-1,2-fucosidase belonging to phylogenetic cluster III of GH95 family and having a catalytic domain characterized by an amino acid sequence having at least 42% sequence identity to amino acids residues 358-730 of SEQ ID NO.: 14 comprising the catalytic domain of AmGH95B,
e. a β-1,3-galactosidase belonging to phylogenetic cluster IV of GH35 family having a catalytic domain characterized by an amino acid sequence having at least 43% sequence identity to amino acids residues 26-379 of SEQ ID NO.: 16 comprising the catalytic domain of AmGH35A
wherein each of said α-1,3-N-acetylgalactosaminidase, α-1,2-fucosidase, and β-1,3-galactosidase has a pI value of >7.5.

2. A blood product comprising the population of enzyme-converted ABO-compatible red blood cells according to claim 1, wherein said cells are reconstituted as a cell suspension in a medium suitable for transfusion.

3. The blood product according to claim 2, wherein said population of enzyme-converted ABO-compatible red blood cells has:
a. a level of detectable A-antigens, B-antigens and extended A- and extended B-antigens on said red blood cells reduced by at least 95% relative to corresponding untreated A or B (or AB) red blood cells, as measured by antibody staining and flow cytometry; or
b. improved compatibility as compared to:
i. corresponding untreated A, B or AB red blood cells, or
ii. enzyme-converted A, B or AB red blood cells obtained from corresponding untreated A, B or AB red blood cells by enzymatic removal of only A-, B-antigens,
wherein said compatibility is tested against a cohort of group O plasmas.

4. The blood product according to claim 2 or 3, for use as a medicament.

5. The blood product according to claim 4, for use in treatment of a human suffering from anemia requiring transfusion.

6. An enzyme composition or kit for conversion of donor red blood cells to enzyme-converted red blood cells according to claim 1, comprising purified enzymes characterized as:
(i) a β-1,3-N-acetylgalactosaminidase belonging to phylogenetic cluster II of GH20 family, and
(ii) a α-1,3-galactosidase belonging to phylogenetic cluster II of GH110 family;
wherein said β-1,3-N-acetylgalactosaminidase and said α-1,3-galactosidase have a pI value of >7.5;
and/or purified enzymes characterized as:
(iii) a α-1,3-N-acetylgalactosaminidase belonging to phylogenetic cluster I of GH36 family or phylogenetic cluster II of GH109 family,
(iv) a α-1,2-fucosidase belonging to phylogenetic cluster III of GH95 family, and
(v) a β-1,3-galactosidase belonging to phylogenetic cluster IV of GH35 family,
wherein each of said α-1,3-N-acetylgalactosaminidase, α-1,2-fucosidase, and β-1,3-galactosidase has a pI value of >7.5.

7. The enzyme composition or kit according to claim 6,
wherein said β-1,3-N-acetylgalactosaminidase belonging to phylogenetic cluster II of the GH20 family has a catalytic domain characterized by an amino acid sequence having at least 61% sequence identity to amino acid residues 164-552 of SEQ ID NO.: 2 comprising the catalytic domain of AmGH20A, and wherein said β-1,3-N-acetylgalactosaminidase has a pI value of >7.5.

8. The enzyme composition or kit according to claim 6 or 7, wherein said α-1,3-galactosidase belonging to phylogenetic cluster II of the GH110 family has a catalytic domain characterized by an amino acid sequence having least 36% sequence identity to amino acid residues 213-795 of SEQ ID NO. 8 comprising the catalytic domain of AmGH110A, and
wherein said α-1,3-galactosidase has a pI value of >7.5.

9. The enzyme composition or kit according to any one of claims 6 to 8, wherein said α-1,3-N-acetylgalactosaminidase belongs to:
(I) phylogenetic cluster I of the GH36 family and has a catalytic domain characterized by an amino acid sequence having at least 36% sequence identity to amino acid residues 385-749 of SEQ ID NO.: 10 comprising the catalytic domain of AmGH36A or
(II) phylogenetic cluster II of the GH109 family and has a catalytic domain characterized by an amino acid sequence having at least 46% sequence identity to amino acids residues residues 27-481 of SEQ ID NO. 12 comprising the catalytic domain of AmGH109B, and
wherein said α-1,3-N-acetylgalactosaminidase has a pI value of >7.5.

10. The enzyme composition or kit according to any one of claims 6 to 9, wherein said α-1,2-fucosidase belonging to phylogenetic cluster III of the GH95 family has a catalytic domain characterized by an amino acid sequence having at least 42% sequence identity to amino acids residues 358-730 of SEQ ID NO.: 14 comprising the catalytic domain of AmGH95B, and
wherein said α-1,2-fucosidase has a pI value of >7.5.

11. The enzyme composition or kit according to any one of claims 6 to 10, wherein said β-1,3-galactosidase belonging to phylogenetic cluster IV of the GH35 family has a catalytic domain characterized by an amino acid sequence having at least 43% sequence identity to amino acids residues 26-379 of SEQ ID NO.: 16 comprising the catalytic domain of AmGH35A, and
wherein said β-1,3-galactosidase has a pI value of >7.5.

12. The enzyme composition or kit according to claim 6,
wherein said β-1,3-N-acetylgalactosaminidase belonging to phylogenetic cluster II of the GH20 family has a catalytic domain characterized by an amino acid sequence having at least 61% sequence identity to amino acid residues 164-552 of SEQ ID NO.: 2 comprising the catalytic domain of AmGH20A;
wherein said α-1,3-galactosidase belonging to phylogenetic cluster II of the GH110 family has a catalytic domain characterized by an amino acid sequence having least 36% sequence identity to amino acid residues 213-795 of SEQ ID NO. 8 comprising the catalytic domain of AmGH110A;
wherein said α-1,3-N-acetylgalactosaminidase belongs to:
phylogenetic cluster I of the GH36 family and has a catalytic domain characterized by an amino acid sequence having at least 36% sequence identity to amino acid residues 385-749 of SEQ ID NO.: 10 comprising the catalytic domain of AmGH36A, or
phylogenetic cluster II of the GH109 family and has a catalytic domain characterized by an amino acid sequence having at least 46% sequence identity to amino acids residues residues 27-481 of SEQ ID NO. 12 comprising the catalytic domain of AmGH109B;
wherein said α-1,2-fucosidase belonging to phylogenetic cluster III of the GH95 family has a catalytic domain characterized by an amino acid sequence having at least 42% sequence identity to amino acids residues 358-730 of SEQ ID NO.: 14 comprising the catalytic domain of AmGH95B;
wherein said β-1,3-galactosidase belonging to phylogenetic cluster IV of the GH35 family has a catalytic domain characterized by an amino acid sequence having at least 43% sequence identity to amino acids residues 26-379 of SEQ ID NO.: 16 comprising the catalytic domain of AmGH35A, and
wherein each of said purified enzymes has a pI value of >7.5.

13. A method for removing A-antigens, B-antigens and extended A- and extended B-antigens from A, B or AB red blood cells comprising the steps of:
(i) contacting A, B or AB red blood cells with the enzyme composition according to any one of claims 6 to 12 under low ionic strength, isotonic and neutral pH conditions at room temperature for a period sufficient to remove the A-antigens, B-antigens and extended A- and extended B-antigens, and
(ii) depleting the product of step (i) for the purified enzymes of said enzyme composition,
wherein said enzyme composition is capable of removing all detectable A-antigens, B-antigens and extended A- and extended B-antigens from said red blood cells.

14. The method according to claim 13, wherein the product of step (i) is depleted for the purified enzymes of said enzyme composition by washing said product in phosphate buffered saline at room temperature.

15. The method according to claim 13 or 14, wherein red blood cells present in the blood product obtained in step (ii) are recovered and reconstituted as a cell suspension in a medium suitable for transfusion.

16. Use of the composition according to any one of claims 6 to 12 for enzymatic removal of A-antigens, B-antigens and extended A- and extended B-antigens from group A, B or AB red blood cells to obtain ABO-universal, compatible red blood cells.
